(19) United States
(12) Patent Application Publication
(10) Pub. No.: US 2019/0106592 A1
(43) Pub. Date: Apr. 11, 2019

(54) HYPERBRANCHED POLYMERS FOR ORGANIC PIGMENT DISPERSIONS AND WATER-SENSITIVE PIGMENTS

(71) Applicant: BASF Coatings GmbH, Muenster-Hiltrup (DE)

(72) Inventors: Emerson Keith Colyer, Whitehouse, OH (US); Abhijit Jadhav, Ann Arbor, MI (US)

(73) Assignee: BASF Coatings GmbH, Muenster-Hiltrup (DE)

(21) Appl. No.: 15/729,701

(22) Filed: Oct. 11, 2017

(51) Int. Cl. C09D 167/04; C08G 83/00; C08G 63/08; C07C 69/712

(52) U.S. Cl. CPC C09D 167/04; C07C 69/712; C08G 63/08; C08G 83/006

(57) ABSTRACT

Aryl-modified hyperbranched polyols are described, as are coating compositions containing these polyols. These polyols enable waterborne coating compositions that: include water-sensitive pigments; are storage stable; and have a low content of VOCs, as well as solventborne coating compositions that: include organic pigments; are storage stable; and enable certain enhanced color properties.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF THE MATERIAL ON THE COMPACT DISC

Not applicable.

### BACKGROUND OF THE INVENTION

### Field of the Invention

The present invention relates to aryl-modified hyperbranched polyols that provide coatings that have excellent flexibility and improved durability due to presence of aryl groups. The present invention also relates to waterborne coating compositions containing the aryl-modified hyperbranched polyols and water-sensitive pigments such as micas and aluminum and solventborne coating compositions containing the aryl-modified hyperbranched polyols and organic pigments.

### Description of the Related Art Including Information Disclosed Under 37 CFR 1.97 and 1.98

This section provides information helpful in understanding the invention but that is not necessarily prior art. All references discussed below are incorporated herein by reference in their entirety.

For waterborne coating compositions containing water-sensitive pigments, such as micas and aluminum, to be storage stable, the water-sensitive pigments must be stabilized to protect them from reactions with water. For instance, aluminum pigments may be stabilized with chromium compounds. However, chromium compounds typically require the addition of a solvent, which inhibits the creation of low volatile organic compound (VOC)-compliant coatings. Chromium compounds also present environmental risks due to their toxicity.

One approach to eliminate the need for chromium compounds is to stabilize the water-sensitive pigments in a solvent borne but water-reducible vehicle. However, suitable polymer solution vehicles typically have a high content of VOCs when rendered to a useable viscosity or are difficult to easily and reproducible disperse into waterborne systems.

For solventborne coating compositions, the pigments used to provide color must be reduced and stabilized at optimal particle sizes, which varies with the pigment, in order for the coatings to exhibit optimal color properties. To obtain optimal color properties when using certain organic pigments, it is necessary to reduce the pigment particle size much more than for typical inorganic pigments. Adsorption characteristics of organic pigments also differ, and thus, affect the color properties.

Typically, resins are used to facilitate the mechanical reduction and subsequent stabilization of the pigment particles. Due to the different physical/chemical nature of organic pigments compared to inorganic pigments and much smaller optimal particle size of organic pigments, the resins used to disperse inorganic pigments are not effective for making optimal organic pigment dispersions. Moreover, resins that are specifically tailored to dispersing organic pigment are expensive.

Colyer, U.S. Patent Application Publ'n No. 2016/0017175, published Jan. 21, 2016, describes a coating composition that includes a flexible hyperbranched polyol preparable by (a) reacting a polyol comprising at least three hydroxyl groups with an aliphatic dicarboxylic acid having from 6 to 36 carbon atoms or an esterifiable derivative of the aliphatic dicarboxylic acid to form a hydroxyl-functional core; (b) reacting the core with a cyclic carboxylic acid anhydride to form a carboxylic acid-functional first intermediate product; and (c) reacting the first intermediate product with an epoxide-functional compound having one epoxide group to form the hyperbranched polyol. The coating composition may be cured to a coating layer having excellent flexibility.

Ramesh, U.S. Pat. No. 6,569,956, issued May 27, 2003, discloses a hyperbranched polyester polyol macromolecule having a plurality of both embedded and exterior hydroxyl groups. The hyperbranched polyol includes a central nucleus, a first chain extension, an intermediate substituent, and a second chain extension. The central nucleus is a hydrocarbon structure with a plurality of oxygen atoms. The first chain extender is attached to the central nucleus and includes a carboxylic ester group and a plurality of hydroxyl groups. The intermediate substituent is attached to the first chain extender and is a polyfunctional carboxylic acid or anhydride. The preferred intermediate substituent is a cyclic compound. The second chain extension is attached to the intermediate substituent. The preferred second chain extension includes a glycidyl ester or epoxy. Also disclosed are coating compositions in which the lower branched polyol is reacted with an aminoplast or with an isocyanate.

Rink, U.S. Pat. No. 6,515,192, issued Feb. 4, 2003, discloses hyperbranched compounds having a tetrafunctional central group of the general formula C[—A_{q}—X—]m[—Aᵣ—X—]ₙ[—Aₛ—X—]ₒ[—Aₜ—X—]ₚ, in which m+n+o+p=4, m is an integer from 1 to 3 and n, o, and p=0 or an integer from 1 to 3; q, r, s, and t are an integer from 1 to 5, where q>r, s, and t; X=—O—, —S—, or —NH—; A=—CR₂—, where R=—H, F, —Cl, —Br, —CN, —NO₂, C₁-C₃ alkyl, C₁-C₃ haloalkyl, or C₂-C₃ alkoxy radical or, if q, r, s, and/or t are at least 2, a C₂-C₄ alkanediyl and/or C₂-C₄ oxaalkanediyl radical which bridges 2 to 5 carbon atoms, and/or an oxygen atom —O—, which bridges 3 to 5 carbon atoms, of the radical —A—.

Ramesh, U.S. Pat. No. 6,646,049, issued Nov. 11, 2003, discloses a binder for a coating composition with a principal resin polyol in combination with a hyper-branched polyol as a reactive intermediate and at least one crosslinker. The principal resin polyol is at least one of a polyester polyol, a polyether polyol, and a polyacrylate. Hyper-branched polyester polyols may be used as reactive diluents, which will cross-link with isocyanates, isocyanurates, epoxides, anhydrides, or their corresponding polyacids and/or aminoplasts to form a binder having particular properties, to help control the rheology of a coating system. The hyperbranched polyol, the principal resin polyol, or both optionally include a carbamate functional group. Coating compositions may be made using the binders together with additional components.

Ramesh et al., U.S. Pat. No. 6,861,150, issued Mar. 1, 2005, discloses a rheology control agent for a coating composition that is the reaction product of a first compound comprising a plurality of hydroxyl groups, of a lactone compound, and of a carbamate compound.

Ramesh et al., U.S. Pat. No. 7,226,971, issued Jun. 5, 2007, discloses a polyester resin for use in a coating composition. The polyester resin is the reaction product of a first compound comprising a plurality of hydroxyl groups, a lactone compound, a carboxylic acid anhydride, an epoxy compound having at least one epoxy group, and a carbamate compound.

Bruchmann et al., U.S. Pat. No. 7,858,733, issued Dec. 28, 2010, discloses high-functionality highly branched or hyperbranched polyesters based on di-, tri-, or polycarboxylic acids and di-, tri-, or polyols, processes for preparing them, and their use in coatings. The high-functionality highly branched or hyperbranched polyesters have a molecular weight Mₙ of at least 500 g/mol and a polydispersity M_{w}/Mₙ of 1.2-50, obtainable by reacting at least one aliphatic, cycloaliphatic, araliphatic, or aromatic dicarboxylic acid (A2) or derivatives thereof and at least one divalent aliphatic, cycloaliphatic, araliphatic, or aromatic alcohol (B2) containing 2 OH groups with either: a) at least one x-valent aliphatic, cycloaliphatic, araliphatic, or aromatic alcohol (Cₓ) containing more than two OH groups, x being a number greater than 2; or b) at least one aliphatic, cycloaliphatic, araliphatic, or aromatic carboxylic acid (D_{y}) or derivatives thereof containing more than two acid groups, y being a number greater than 2, in each case optionally in the presence of further functionalized building blocks E and c) subsequently reacting the product, if appropriate, with a monocarboxylic acid F, and the ratio of the reactive groups in the reaction mixture being chosen so as to set a molar ratio of OH groups to carboxyl groups or derivatives thereof of from 5:1 to 1:5, preferably from 4:1 to 1:4, more preferably from 3:1 to 1:3 and very preferably from 2:1 to 1:2.

It remains desirable to make further improvements in coating compositions containing hyperbranched polyols to provide coating compositions and coatings with excellent properties. One object of the present invention is to provide waterborne coating compositions that: include water-sensitive pigments; are storage stable; and have a low content of VOCs. These waterborne coating compositions allow the water-sensitive pigments to be safely dispersed without the use of chromium compounds. Another object of the present invention is to provide solventborne coating compositions that: include small organic pigments; are storage stable; and exhibit optimal color properties. These solventborne coating compositions are an economically viable alternative to specialty resins that tailored to dispersing organic pigments, while being more capable of stabilizing fine organic pigment particles than conventional dispersing resins.

### BRIEF SUMMARY OF THE INVENTION

The objects of the present invention are achieved with aryl-modified hyperbranched polyols and coating compositions and coatings containing these polyols.

As detailed in FIG. 1, the aryl-modified hyperbranched polyols are preparable by, for example:
(a) reacting a portion of hydroxyl functional groups on a core comprising hydroxyl functional groups with an aromatic carboxylic acid of the formula (I):

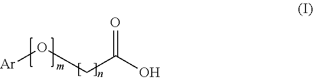

wherein: Ar is an optionally substituted aryl group; m is 0 or 1; and n is 0 or an integer from 1 to 8,
to obtain a first intermediate product still comprising at least one hydroxyl functional group; and then,
   (b1) reacting the remaining hydroxyl functional groups on the first intermediate product with lactone(s), to obtain an aryl-modified hyperbranched polyol with polylactone chain extensions for solventborne coating compositions; or
   (b2) reacting the remaining hydroxyl functional groups on the first intermediate product with a cyclic carboxylic acid anhydride to form a carboxylic acid-functional second intermediate product comprising carboxylic acid functional groups; and then,
   (c1) reacting essentially all the carboxylic acid functional groups of the second intermediate product with an epoxide-functional compound having one epoxide group, to obtain an aryl-modified hyperbranched polyol comprising hydroxyl functional groups which is useful for solventborne coating compositions; or
   (c2) reacting a portion of the carboxylic acid functional groups of the second intermediate product with an epoxide-functional compound having one epoxide group, to obtain an aryl-modified hyperbranched polyol comprising carboxylic acid functional groups and hydroxyl functional groups which is useful for waterborne coating compositions; and optionally,
      (c2a) reacting hydroxyl functional groups of the aryl-modified hyperbranched polyol comprising carboxylic acid functional groups and hydroxyl functional groups with lactone(s), to obtain an aryl-modified hyperbranched polyol comprising carboxylic acid functional groups and polylactone chain extensions for waterborne coating compositions and; or
   (c3) reacting essentially all the carboxylic acid functional groups of the second intermediate product with a polyol comprising at least three hydroxyl groups, to obtain a third intermediate product comprising hydroxyl functional groups; and then,
   (d1) reacting the third intermediate product with a lactone(s), to obtain an aryl-modified hyperbranched polyol with polylactone chain extensions for solventborne coating compositions; or
   (d2) reacting a portion of the hydroxyl functional groups on the third intermediate product with a cyclic carboxylic acid anhydride to form a fourth intermediate product comprising carboxylic acid functional groups and hydroxyl functional groups which is useful for waterborne coating compositions; and optionally,
      (d2a) reacting the hydroxyl functional groups of the fourth intermediate product with lactone(s), to obtain an aryl-modified hyperbranched polyol comprising carboxylic acid functional groups and polylactone chain extensions for waterborne coating compositions and with polylactone chain extensions; or
   (d3) reacting the remaining hydroxyl functional groups on the third intermediate product with a cyclic carboxylic acid anhydride to form a fourth intermediate product comprising carboxylic acid functional groups; and then,
   (e1) reacting essentially all the carboxylic acid functional groups of the fourth intermediate product with an epoxide-functional compound having one epoxide group, to obtain an aryl-modified hyperbranched polyol comprising hydroxyl functional groups which is useful for solventborne coating compositions; and optionally,
      (e1a) reacting hydroxyl functional groups of the aryl-modified hyperbranched polyol comprising carboxylic acid functional groups and hydroxyl functional groups with lactone(s), to obtain an aryl-modified hyperbranched polyol comprising carboxylic acid functional groups and polylactone chain extensions for waterborne coating compositions; or
   (e2) reacting a portion of the carboxylic acid functional groups of the fourth intermediate product with an epoxide-functional compound having one epoxide group, to obtain an aryl-modified hyperbranched polyol comprising carboxylic acid functional groups and hydroxyl functional groups which is useful for waterborne coating compositions; and optionally,
      (e2a) reacting hydroxyl functional groups of the aryl-modified hyperbranched polyol comprising carboxylic acid functional groups and hydroxyl functional groups with lactone(s), to obtain an aryl-modified hyperbranched polyol comprising carboxylic acid functional groups and polylactone chain extensions for waterborne coating compositions,
wherein the core comprising hydroxyl functional groups is a polyol comprising at least three hydroxyl groups or a reaction product obtained by reacting a polyol comprising at least three hydroxyl groups with an aliphatic dicarboxylic acid having from 6 to 36 carbon atoms, an aromatic dicarboxylic acid having from 8 to 20 carbon atoms, an esterifiable derivative of the aliphatic or aromatic dicarboxylic acid, or a polyurethane diacid according to the following formula:

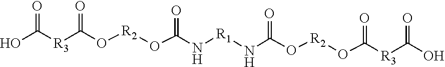

wherein:
R₁ and R₂ are each a C₂-C₁₆ aliphatic group, preferably a C₃-C₈ aliphatic group, and especially a C₃-C₆ aliphatic group; and
R₃ is a C₂-C₂₂ substituted or unsubstituted aliphatic, alicyclic, or aromatic group, preferably a C₆-C₁₈ alicyclic or aromatic group.

In certain embodiments where the core is a reaction product of the polyol and the dicarboxylic acid, due to the physical characteristics of reaction product variations it may be advantageous to conduct the reaction of the dicarboxylic acid and the aromatic carboxylic acids of the formula (I) with the starting polyols simultaneously to obtain the first intermediate product.

When the core comprising hydroxyl functional groups is a reaction product of the polyol and the dicarboxylic acid, the ratio of moles of the polyol to moles of the dicarboxylic acid or esterifiable derivative of the dicarboxylic acid is from about 2.0 to about 2.5 moles of the polyol per mole of the dicarboxylic acid or esterifiable derivative of the dicarboxylic acid. Particularly preferably, on average about one hydroxyl group of each polyol molecule is reacted with the dicarboxylic acid to form the core comprising hydroxyl functional groups. Esterifiable derivatives of the dicarboxylic acids having from 6 to 36 carbon atoms include their anhydrides, acid chlorides, and esterifiable esters.

In step (a), the equivalent ratio of hydroxyl groups of the core comprising hydroxyl functional groups to carboxylic acid groups of the aromatic carboxylic acid of formula (I) is from about 1.0 to about 2.0 equivalents of hydroxyl groups per carboxylic acid group, with the proviso that at least one hydroxyl group of the core remain following step (a). Generally, the number of moles of the aromatic carboxylic acid is adjusted to leave approximately one or two unreacted hydroxyl groups per mole of the first intermediate. If the core is a polyol, the equivalent ratio (OH:COOH) is about 1.5 when the polyol is triol, as low as 1.33 when the polyol is a tetrol, as low as 1.25 when the polyol is a pentol, as low as 1.2 when the polyol is a hexol, and as low as 1.14 when the polyol is an octol. If the core is based on the reaction product of the polyol and the dicarboxylic acid, the number of moles of the aromatic carboxylic acid may be adjusted to leave approximately two unreacted hydroxyl groups per mole of the first intermediate. Thus, the equivalent ratio (OH:COOH) is about 2.0 when the core is based on a dicarboxylic acid and a triol, about 1.5 when the core is based on a dicarboxylic acid and a tetrol, about 1.33 when the core is based on a dicarboxylic acid and a pentol, and about 1.25 when the core is based on a dicarboxylic acid and a hexol. The hydroxyl functionality of the first intermediate may be increased by reacting terminal hydroxyl groups with dimethylol propionic acid, for example.

In step (b2), the equivalent ratio of hydroxyl groups of first intermediate product to anhydride groups of the cyclic carboxylic acid anhydride is from about 1.0 to about 1.25 equivalents of hydroxyl groups per carboxylic anhydride groups. If step (b2) is performed, substantially all the remaining hydroxyl groups of the first intermediate product are reacted with an anhydride group in step (b2).

In steps (c1) and (c2), the equivalent ratio of carboxylic acid groups of the second intermediate product to epoxide groups of the epoxide-functional compound is from about 1.0 to about 2.5 equivalents of carboxylic acid groups per equivalents epoxide groups. For the organic solvent-based coating compositions obtained in step (c1), the equivalent ratio of carboxylic acid groups of the second intermediate product to epoxide groups of the epoxide-functional compound is preferably from about 1.0 to about 1.1 equivalents of carboxylic acid groups per equivalents epoxide groups. For the waterborne coating compositions obtained in step (c2), the equivalent ratio carboxylic acid groups of the second intermediate product to epoxide groups of the epoxide-functional compound in step c2 is about 1.75 to about 2.25 equivalents of carboxylic acid groups per equivalents epoxide groups such that unreacted carboxylic acid groups remain. The unreacted carboxylic acid groups are at least partially neutralized with a base, which results in the aryl-modified hyperbranched polyol being easily water-reducible.

In step (c3), the ratio of moles of the polyol to moles of the second intermediate is from about 2.0 to about 2.5 moles of the polyol per mole of second intermediate product. Particularly preferably, on average about one hydroxyl group of each polyol molecule is reacted with second intermediate product. Generally, an equimolar amount of the polyol monomers is esterified with the available carboxylic acid functional groups of the second intermediate product, such that the third intermediate has from 4 to 14 hydroxy functional groups (e.g., 2 moles of triol with 2 carboxylic acid functional groups, yields 4 hydroxy functional groups). If necessary, the hydroxyl functionality of the third intermediate product may be increased by reacting a terminal hydroxyl group with dimethylol propionic acid, for example.

In steps (b1), (c2a), (d1), and (d2a), the available hydroxyl functional groups of the first intermediate reaction product in step (b1), the available hydroxyl functional groups of the aryl modified hyperbranched polyol comprising carboxylic acid functional groups and hydroxyl functional groups in step (c2a), the available hydroxyl functional groups of the third intermediate reaction product in step (d1), and the available hydroxyl functional groups of the fourth intermediate reaction product in step (d2a) react with lactone(s) to produce poly-lactone chains that grow from each available hydroxyl group. The number of moles of lactone relative to the moles of the first intermediate reaction product in step (b1), the aryl modified hyperbranched polyol comprising carboxylic acid functional groups and hydroxyl functional groups in step (c2a), the third intermediate reaction product in step (d1), and the fourth intermediate reaction product in step (d2a) will determine the degree of extension of the poly-lactone chains. The length of the poly-lactone chains is preferably between 5 and 30 units.

The coating composition may be solventborne (organic solvent-based) or waterborne.

The aryl-modified hyperbranched polyols obtained in steps (c2), (c2a), (d2), (d2a), (e2), and (e2a) are suitable for waterborne coating compositions that contain water-sensitive pigments because neutralization of the unreacted carboxylic acid groups that remain with a base renders them water-reducible.

The aryl-modified hyperbranched polyols obtained in steps (b1), (e1), (d1), (e1), and (e1a) are suitable for solventborne coating compositions containing organic pigments because the aryl groups appended to the compact core have an affinity for the surface of the organic pigments, particularly when their structures are similar. In effect, the aryl groups serve as anchor sites to the organic pigment, while the poly-lactone chains (or the hydroxyl groups derived from the epoxide in the case of c1) extend out from the compact core and into the continuous phase of the organic pigment dispersion. Steric hindrance due to the poly-lactone chains inhibits agglomeration of the organic pigments, which improves storage stability and improves certain color performance parameters.

Coatings produced from the coating compositions containing the aryl-modified hyperbranched polyols have excellent durability due to the incorporation of the aryl groups and excellent flexibility, particularly at low temperatures. The aryl-modified hyperbranched polymers obtained in steps (c2), (c2a), (d2), (d2a), (e2), and (e2a) are suitable for waterborne coating compositions that: include water-sensitive pigments; are storage stable; and have a low content of VOCs. The aryl-modified hyperbranched polymers obtained in steps (b1), (e1), (d1), (e1), and (e1a) are suitable for solventborne coating compositions that: include organic pigments; are storage stable; and exhibit optimal color properties.

### BRIEF DESCRIPTION OF THE SEVERAL VIEWS OF THE DRAWINGS

FIG. 1 shows the reaction pathways that provide the aryl-modified hyperbranched polyols according to the present invention.

FIG. 2 shows the structure of the aryl-modified hyperbranched polyol produced in Example 5.

FIG. 3 shows panels from powerwash adhesion testing of <250 gm/L VOC waterborne basecoat of inventive Example 1 with GLASURIT LINE-90 as a control.

FIG. 4 shows panels from Ford immersion adhesion testing of <250 gm/L VOC waterborne basecoat of inventive Example 1 with GLASURIT LINE-90 as a control.

### DETAILED DISCLOSURE

A detailed description of exemplary, nonlimiting embodiments follows.

“A,” “an,” “the,” “at least one,” and “one or more” are used interchangeably to indicate that at least one of the item is present; the indefinite articles indicate a plurality of such items may be present unless the context clearly indicates otherwise.

All references, patents, applications, tests, standards, documents, publications, brochures, texts, articles, etc. mentioned herein are incorporated herein by reference.

All disclosures of ranges include the endpoints of the ranges and are disclosures of all values and further divided ranges within the entire range. Also, all values and subranges within a numerical limit or range are specifically included as if explicitly written out. For example, C₂-C₂₂ includes C₂ and C₂₂ as well as, e.g., C₃, C₁₂, C₁₇, etc.

As used herein, the phrase “a compound that may be obtained by reaction . . . ” and the like is not limited by the noted reaction and refers to a chemical product capable of being obtained by the noted reaction but not necessarily being so obtained. As is generally known in the art, there typically exists more than one synthetic pathway to a given compound, such pathways being readily envisioned by those of ordinary skill in the art given the noted reaction and/or structure of the compound and/or its reactants. In all events the compounds described herein, whether described by chemical formula or by a reaction scheme, are fully described and enabled chemical compounds, and need not be associated with a method for making.

In this description of the invention, for convenience, “polymer” and “resin” are used interchangeably to encompass resins, oligomers, and polymers.

The terms “comprising,” “including,” “containing,” and “having” are inclusive and therefore specify the presence of stated items, but do not preclude the presence of other items. As used in this specification, the term “or” includes any and all combinations of one or more of the listed items.

Aryl-modified hyperbranched polyols—in general:

The aryl-modified hyperbranched polyol of this invention can be prepared by appending a limited number of aromatic carboxylic acid groups of the formula (I):

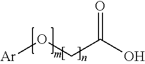

wherein: Ar is an optionally substituted aryl group; m is 0 or 1; and n is 0 or an integer from 1 to 8),
   onto a multi-hydroxyl functional core, then subsequently appending to or extending from the aryl-modified polyol core, other functional and/or structural moieties selected to achieve whichever embodiment of the invention is desired.

The hydroxy-functional core onto which the aromatic carboxylic acid units are appended can be a polyol compound such as trimethylolpropane, pentaerythritol, dipentaerythritol, or tripentaerythritol, for example, or, alternately, the hydroxy-functional core can be the reaction product of such polyols with a multi-functional compound capable of linking two or more of these polyols together. Suitable linking components include aliphatic dicarboxylic acids having from 6 to 36 carbon atoms, aromatic dicarboxylic acids having from 8 to 20 carbon atoms, esterifiable derivatives of the aliphatic or aromatic dicarboxylic acids, or multi-acid-functional compounds derived from other multi-functional components such as diols or polyols.

### Synthesis:

The aryl-modified hyperbranched polyol can be prepared by a synthesis having a step (a) of reacting a polyol comprising at least three hydroxyl groups, or alternatively, a suitable hydroxy-functional intermediate as described in alternative step (a₀), with an aromatic carboxylic acid of the formula (I):

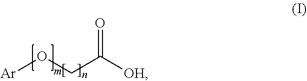

wherein: Ar is an optionally substituted aryl group, which is a monocyclic or condensed polycyclic aromatic hydrocarbon group; m is 0 or 1; and n is 0 or an integer from 1 to 8,
   to obtain a first intermediate product comprising hydroxyl functional groups.

In certain embodiments, Ar is an optionally substituted monocyclic aromatic hydrocarbon group, preferably a phenyl group optionally bearing from one to three substituents selected from the group consisting of a straight or branched chain alkyl of 1 to 6 carbon atoms, an alkoxy of 1 to 6 carbon atoms, alkoxy alkyl wherein the alkyl moieties have 1 to 6 carbon atoms, halo, phenyl, and hydroxy. In certain embodiments, Ar represents an optionally substituted condensed polycyclic aromatic hydrocarbon group, preferably a naphthyl, anthranyl, or phenanthryl group optionally bearing from one to three substituents selected from the group consisting of a straight or branched chain alkyl of 1 to 6 carbon atoms, an alkoxy of 1 to 6 carbon atoms, alkoxy alkyl wherein the alkyl moieties have 1 to 6 carbon atoms, halo, phenyl, and hydroxy.

The polyol of step (a) may preferably be selected from triols, dimers of triols, tetrols, dimers tetrols, and sugar alcohols. Nonlimiting examples of suitable polyols having three or more hydroxyl groups include glycerol, trimethylolmethane, trimethylolethane, trimethylolpropane, 2,2,3-trimethylolbutane-1,4-diol, 1,2,4-butanetriol, 1,2,6-hexanetriol, tris(hydroxyethyl)amine, tris(hydroxyethyl)amine, tris(hydroxypropyl)amine, erythritol, pentaerythritol, diglycerol, triglycerol or higher condensates of glycerol, di(trimethylolpropane), di(pentaerythritol), tri(pentaerythritol), pentaerythritol ethoxylate, pentaerythritol propoxylate, trishydroxymethyl isocyanurate, tris(hydroxyethyl) isocyanurate (THEIC), tris(hydroxypropyl) isocyanurate, inositols or sugars, such as glucose, fructose or sucrose, for example, sugar alcohols such as xylitol, sorbitol, mannitol, threitol, erythritol, adonitol (ribitol), arabitol (lyxitol), xylitol, dulcitol (galactitol) isomalt, polyetherols with a functionality of three or more, based on alcohols with a functionality of three or more reacted with ethylene oxide, propylene oxide, and/or butylene oxide.

In certain preferred embodiments, the polyol of step (a) is at least one of erythritol, pentaerythritol, di(pentaerythritol), trimethylolethane, trimethylolpropane, trimethylolbutane, glycerol, di(trimethylolethane), di(trimethylolpropane), tri(pentaerythritol), pentaerythritol ethoxylate, and pentaerythritol propoxylate. In one particular embodiment, the polyol of step (a) is trimethylolpropane, pentaerythritol, di(trimethylolpropane), di(pentaerythritol), or tri(pentaerythritol).

In a variant, a suitable hydroxy-functional intermediate for step (a) can be prepared by a synthesis having a step (a₀) of reacting a polyol comprising at least three hydroxyl groups with an aliphatic dicarboxylic acid having from 6 to 36 carbon atoms, an aromatic dicarboxylic acid having from 8 to 20 carbon atoms, an esterifiable derivative of the aliphatic or aromatic dicarboxylic acid, or a polyurethane diacid according to the following formula:

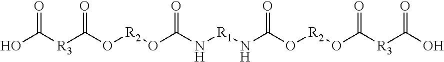

wherein:
R₁ and R₂ are each a C₂-C₁₆ aliphatic group, preferably a C₃-C₈ aliphatic group, and especially a C₃-C₆ aliphatic group; and
R₃ is a C₂-C₂₂ substituted or unsubstituted aliphatic, alicyclic, or aromatic group, preferably a C₆-C₁₈ alicyclic or aromatic group,
   to form the alternative hydroxyl-functional intermediate for step (a).

The aliphatic dicarboxylic acid having from 6 to 36 carbon atoms for step (a₀) or the esterifiable derivative of the aliphatic dicarboxylic acid may be linear, branched, or cyclic, with the proviso that cyclic dicarboxylic acids include a noncyclic segment of at least about 6 carbon atoms. Nonlimiting examples of suitable dicarboxylic acids include adipic acid, suberic acid, azelaic acid, sebacic acid, undecanedioic acid (brassylic acid), dodecanedioic acid, traumatic acid, hexadecanedioic acid (thapsic acid), octadecanedioic acid, tetradecanedioic acid, and dimer fatty acids having 36 carbon atoms. In various embodiments, α,ω-dicarboxylic acids and dimer fatty acids having 36 carbon atoms are preferred.

It is known that dimer fatty acids having 36 carbon atoms may have multiple isomers. Dimer fatty acids are commercially available, for example from BASF under the trademark EMPOL®, from Arizona Chemical under the trademark UNIDYME™, from Croda International Plc under the trademark Pripol™, and from Emery Oleochemicals as EMERY® Dimer Acids. Esterifiable derivatives of the dicarboxylic acids having from 6 to 36 carbon atoms include their mono- or diesters with aliphatic alcohols having 1 to 4 carbon atoms, preferably the methyl and ethyl esters, as well as the anhydrides.

Non-limiting examples of the aromatic dicarboxylic acid having 8 to 20 carbon atoms are terephthalic acid, isophthalic acid, 1,5-nathphalenedicarboxylic acid, 2,6-nathphalenedicarboxylic acid, and 2,7-nathphalenedicarboxylic acid.

The aliphatic or aromatic dicarboxylic acid of step (a₀) is reacted with a polyol comprising at least three hydroxyl groups. The hydroxyl groups of the polyol can be primary, secondary, and/or tertiary hydroxyl groups.

The polyols of step (a₀) are the same as those used in step (a). In one particular embodiment, the polyol of step (a₀) is trimethylolpropane, pentaerythritol, di(trimethylolpropane), tri(pentaerythritol), or di(pentaerythritol).

In various examples, the ratio in step (a₀) of moles of the polyol to moles of the dicarboxylic acid (or esterifiable derivative of the dicarboxylic acid) is from about 2.0 to about 2.5, preferably from about 2.0 to about 2.2, and more preferably from about 2.0 to about 2.07 moles of the polyol per mole of the dicarboxylic acid (or esterifiable derivative of the dicarboxylic acid). Particularly preferably, on average about one hydroxyl group of each polyol molecule is reacted with the dicarboxylic acid (or esterifiable derivative of the dicarboxylic acid) in step (a₀).

The polyurethane diacid of step (a₀) has the formula:

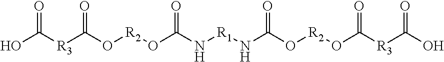

wherein:
R₁ and R₂ are each a C₂-C₁₆ aliphatic group, preferably a C₃-C₈ aliphatic group, and especially a C₃-C₆ aliphatic group; and
R₃ is a C₂-C₂₂ substituted or unsubstituted aliphatic, alicyclic, or aromatic moiety, preferably a C₆-C₁₈ alicyclic, or aromatic moiety.

In various examples, the diacid of step (a₀) has the following formulae:

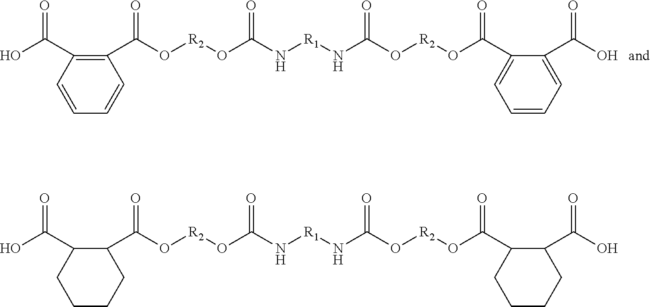

wherein:
R₁ is a C₄-C₈ aliphatic group, especially a C₆ aliphatic group; and
R₂ is a C₂-C₄ aliphatic group, especially a C₃ aliphatic group.

The esterification reaction in steps (a) and (a₀) may be carried out by known, standard methods. For example, this reaction is conventionally carried out at temperatures of between about 180° C. and about 280° C. in the presence, if desired, of an appropriate esterification catalyst. Typical catalysts for the esterification polymerization are protonic acids and Lewis acids, for example sulfuric acid, para-toluenesulfonic acid, sulfates, and hydrogen sulfates, such as sodium hydrogen sulfate, phosphoric acid, phosphonic acid, hypophosphorous acid, titanium alkoxides, and dialkyltin oxides, for example dibutyltin oxide, dibutyltin dilaurate, lithium octanoate, under reflux with small quantities of a suitable solvent as entraining agent such as an aromatic hydrocarbon, for example xylene, or a (cyclo)aliphatic hydrocarbon, for example cyclohexane. As a non-limiting, specific example, the polyester may include stannous octoate or dibutyltin oxide. An acidic inorganic, organometallic, or organic catalyst can be used in an amount from 0.1% to 10% by weight, preferably from 0.2% to 2% by weight, based on total weight of the reactants. It may be desirable to carry out the reaction in steps (a) and (a₀) free of catalyst to avoid or minimize side reactions during subsequent steps.

The esterification reaction in steps (a) and (a₀) can be carried out in bulk or in the presence of a solvent that is nonreactive toward the reactants. Nonlimiting examples of suitable solvents include hydrocarbons such as paraffins or aromatics. In some embodiments it may be preferred to use n-heptane, cyclohexane, toluene, ortho-xylene, meta-xylene, para-xylene, xylene isomer mixtures, ethylbenzene, chlorobenzene and ortho- and meta-dichlorobenzene. Other solvents that may be used in the absence of acidic catalysts are ethers, such as dioxane tetrahydrofuran, for example, and ketones such as methyl ethyl ketone and methyl isobutyl ketone, for example. The solvent may be used to aid in removing by-product of the esterification reaction azeotropically.

The amount of solvent that can be used may be at least 0.1% by weight or at least 1% by weight or at least 5% by weight, based on the weight of the starting reactants. Higher amounts of solvent may be used, but it is preferred to keep the concentration of reactants high enough to permit the reaction to be carried out in a commercially viable length of time. Examples of ranges of the solvent that may be employed are from 0.1% to about 30% by weight, or from about 1% to about 15% by weight, or from about 5% to about 10% by weight, based in each case on the weight of the starting reactants.

The esterification reaction in steps (a) and (a₀) may be carried out in the presence of a water-removing agent, for example molecular sieves, especially molecular sieve 4 Å, MgSO₄ and Na₂SO₄.

The esterification reaction in steps (a) and (a₀) may be carried out at temperatures of 60° C. to 250° C., for example at temperatures of 100° C. to 240° C. In certain embodiments the reaction of steps (a) and (a₀) may be carried out at temperatures of 150° C. to 235° C. The reaction time depends upon known factors, which include temperature, concentration of reactants, and presence and identity of catalyst, if any. Typical reaction times may be from about 1 to about 20 hours.

To minimize final volatile organic content, as much of the solvent used to azeotrope the byproduct from steps (a) and (a₀) as is practical may be removed after completion of the reaction of steps (a) and (a₀). Small amounts of solvents selected for their performance in the final resin can be used throughout the rest of the synthesis, for example as a flush following a reagent addition. Solvents that can react with anhydrides or epoxides, such as active hydrogen-containing compounds like hydroxy-functional solvents (e.g., alcohols and monoethers of glycols), are preferably avoided during steps (a) and (a₀) and subsequent reaction steps. After step (a), except for certain embodiments of the invention, the reaction temperature is preferably kept below at temperature at which condensation-type esterification reactions could take place, for example kept below 150° C., for the remainder of the synthesis to minimize the chance of condensation-type esterification reactions which, at this stage of the synthesis, would have undesirable effects on the molecular weight and architecture. For example, further esterification could produce unwanted branching or an undesirably increased molecular weight. The reaction temperature for steps subsequent to steps (a) may be kept below 145° C., below 140° C., or even below 135° C. or 130° C. depending on whether a catalyst is used during steps (a) and (a₀) and the nature of any catalyst used.

In certain embodiments, the aromatic carboxylic acid of step (a) is represented by formulae (II) or (III):

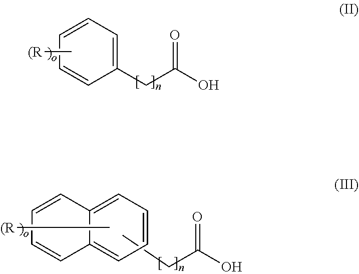

wherein, in formulae (II) and (III), n is 0 or an integer from 1 to 8, preferably from 0 or an integer from 1 to 4, particularly 0 or 1; o is 0 or an integer from 1 to 3; and each R, if present, is independently selected from the group consisting of a straight or branched chain alkyl of 1 to 6 carbon atoms, an alkoxy of 1 to 6 carbon atoms, and an alkoxy alkyl wherein the alkyl moieties have 1 to 6 carbon atoms, halo, phenyl, and hydroxy. Examples of compounds according to formulae (II) and (III) are illustrated below:

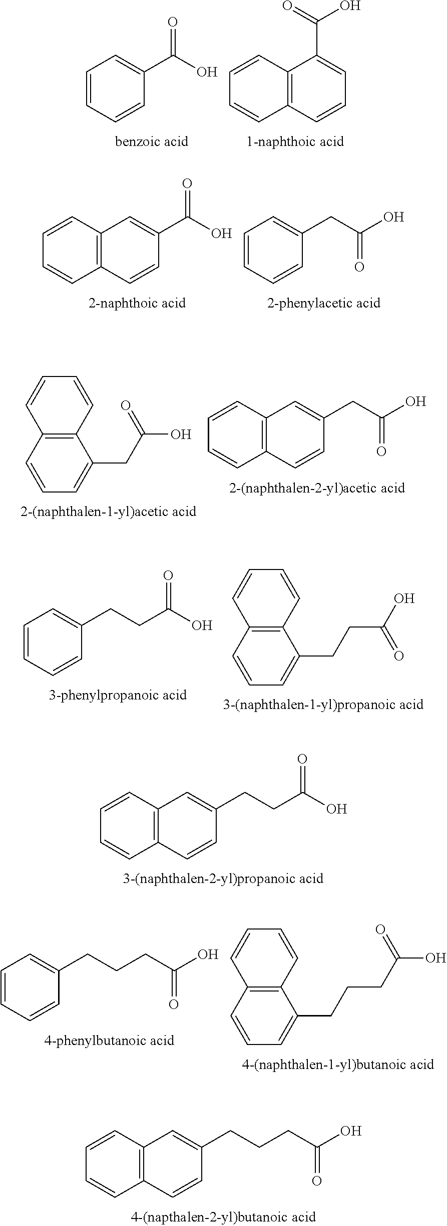

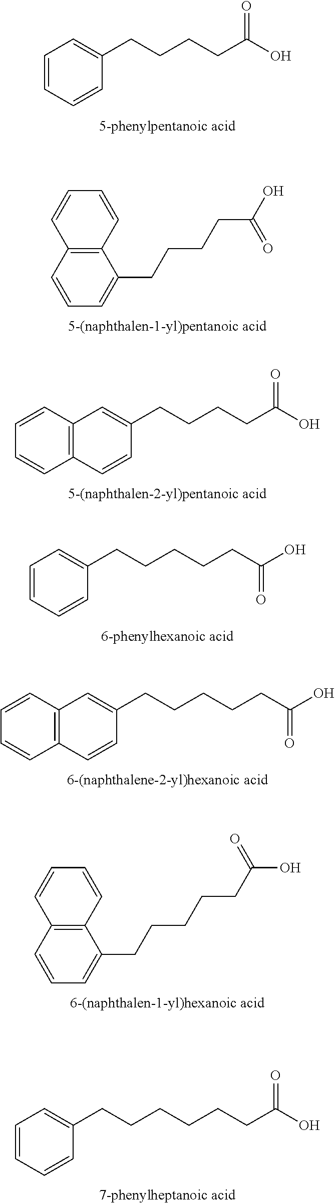

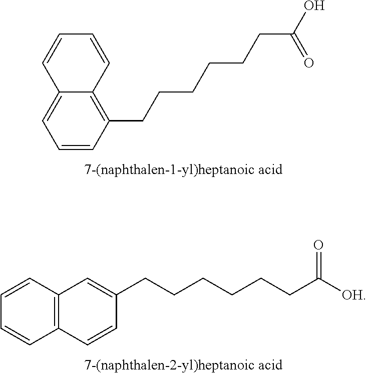 [SMILES: O=C(O)CCCCCCc1cccc2ccccc12 ; O=C(O)CCCCCCc1ccc2ccccc2c1]

In certain embodiments, the aromatic carboxylic acid of step (a) is an aryloxy aromatic carboxylic acid represented by formulae (IV) or (IV):

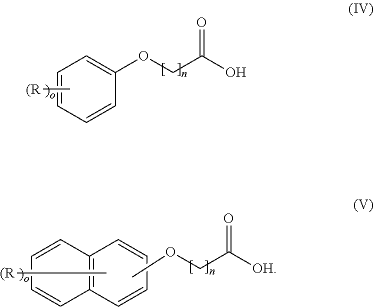

wherein, in formulae (IV) and (V), n is 0 or an integer from 1 to 8, preferably from 0 or an integer from 1 to 4, particularly 0 or 1; o is 0 or an integer from 1 to 3; and each R, if present, is independently selected from the group consisting of a straight or branched chain alkyl of 1 to 6 carbon atoms, an alkoxy of 1 to 6 carbon atoms, alkoxy alkyl wherein the alkyl moieties have 1 to 6 carbon atoms, halo, phenyl, and hydroxy. Examples of compounds according to formulae (IV) and (V) are illustrated below:

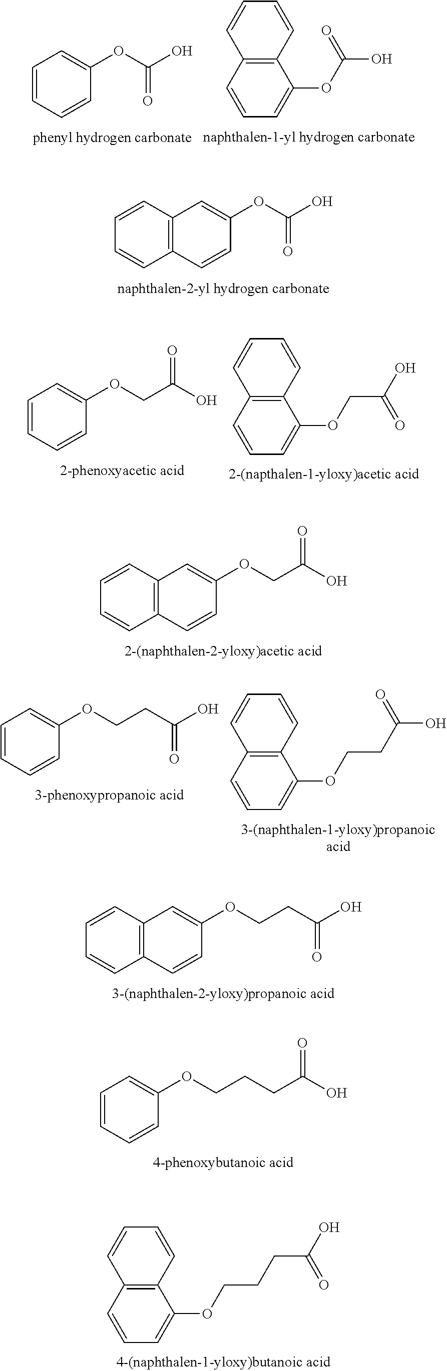

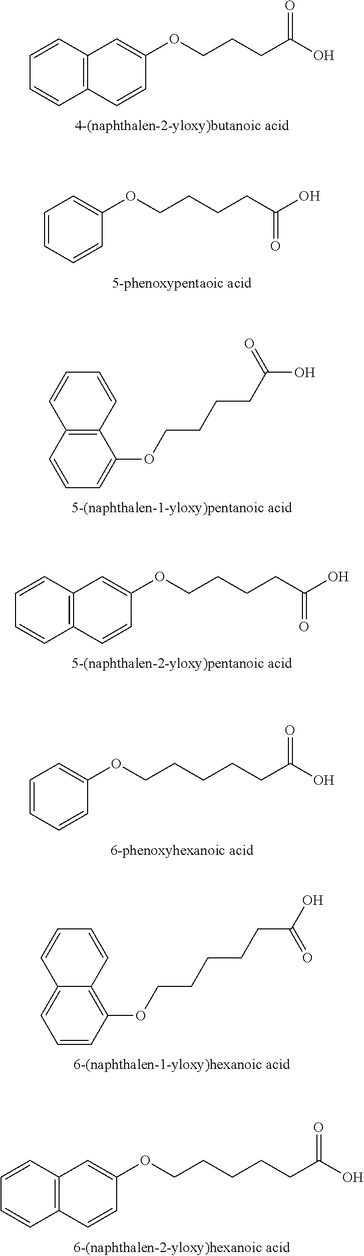

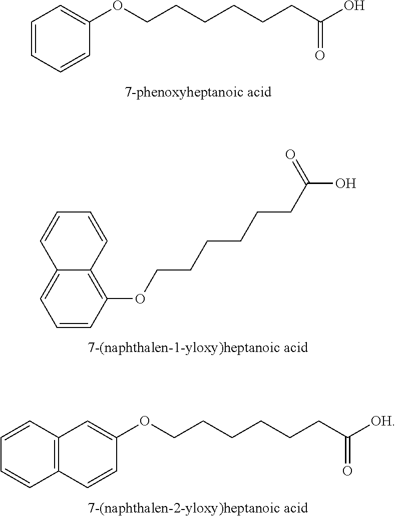 [SMILES: O=C(O)CCCCCCOc1ccccc1 ; O=C(O)CCCCCCOc1cccc2ccccc12 ; O=C(O)CCCCCCOc1ccc2ccccc2c1]

The carboxylic acid functional group reacts with at least one of the hydroxyl groups of the polyol or hydroxy-functional intermediate cores to form the first intermediate product comprising hydroxyl functional groups. The esterification reaction of step (a) is performed using the same methods and conditions as the esterification reaction of step (a₀).

In various embodiments, the equivalent ratio of hydroxyl groups of the polyol or core to carboxylic acid groups of the aromatic carboxylic acid of formula (I) in step (a) is from about 1.0 to about 2.0 equivalents of hydroxyl groups per carboxylic acid group, with the proviso that hydroxyl groups of the core remain following step (a). Particularly preferably, the number of moles of the aromatic carboxylic acid is adjusted to leave approximately one or two unreacted hydroxyl groups per mole of the first intermediate. Thus, the equivalent ratio (OH:COOH) is about 2.0, when the core is based on triols, about 1.5 when the core is based on tetrols, about 1.33 when the core is based on pentols, and about 1.25 when the core is based on hexols. If necessary, the hydroxyl functionality of the first intermediate may be increased by reacting terminal hydroxyl groups with dimethylol propionic acid.

It is possible to conduct the esterification reactions of step (a) and step (a₀) simultaneously by reacting the polyol, the aliphatic or aromatic dicarboxylic acid (or an esterifiable derivative of the aliphatic or aromatic dicarboxylic acid), and the aromatic carboxylic acid of formula (I).

Next, for some embodiments, the remaining hydroxyl functional groups of the first intermediate product prepared in step (a), including any which may have utilized a step (a₀), are reacted in step (b1) with a lactone or lactone mixture to append polylactone chains which extend from the aryl-modified core to produce an aryl-modified hyperbranched polyol with polylactone chain extensions.

Non-limiting examples of the lactones used in step (b1) include ε-caprolactone, γ-caprolactone, β-butyrolactone, β-propriolactone, γ-butyrolactone, α-methyl-γ-butyrolactone, β-methyl-γ-butyrolactone, γ-valerolactone, δ-valerolactone, γ-decanolactone, δ-decanolactone, γ-nonanoic lactone, γ-octanoic lactone. Preferred are ε-caprolactone, γ-caprolactone, δ-valerolactone, and δ-butyrolactone. Particularly preferred are blends of ε-caprolactone and δ-valerolactone. Useful catalysts include those mentioned above for the esterification steps (a) and (a₀).

In step (b1), a ring-opening polymerization of the lactone monomers, first, onto the hydroxyl functionality on the first intermediate product, and then repeatedly onto the hydroxyl functionality created with each ring-opening reaction results in poly-lactone stabilization chains being grown from each hydroxyl group available on the aryl-modified first intermediate reaction product. The number of moles of lactone relative to the moles of the first intermediate product will determine the degree of extension of the poly-lactone chains. Due to the structural similarity of the aryl groups of the aryl-modified hyperbranched polyol to the aryl structure in typical organic pigments, the aryl-modified hyperbranched polyols are reasonably anchored to the surface of the organic pigments. In solventborne coating compositions, the poly-lactone stabilization chains of the invention extend into the continuous phase of the composition and sterically hinder the agglomeration of organic pigments.

For other embodiments, substantially, the remaining hydroxyl functional groups on the first intermediate product prepared in step (a), including any that may have utilized a step (a₀), are reacted in step (b2) with a cyclic carboxylic acid anhydride to form a carboxylic acid-functional second intermediate product comprising carboxylic acid functional groups. The cyclic carboxylic acid anhydride reacts with at least one of the hydroxyl groups of the hydroxyl-functional first intermediate product to form the second intermediate product having at least one carboxylic acid functional group. Preferably, the cyclic carboxylic acid anhydride is reacted with all or substantially all the hydroxyl groups of the first intermediate product to form the second intermediate product. The cyclic carboxylic acid anhydride reacted in step (b2) may be either an aromatic or aliphatic cyclic anhydride.

In certain embodiments, the cyclic carboxylic acid anhydride of step (b2) is at least one of maleic anhydride, hexahydrophthalic anhydride, methylhexahydrophthalic anhydride, tetrahydrophthalic anhydride, phthalic anhydride, succinic anhydride, trimellitic anhydride, methyltetrahydrophthalic anhydride, adipic anhydride, glutaric anhydride, malonic anhydride, itaconic acid anhydride, 5-methyl-5-nobornenedicarboxylic acid anhydride, 1,2-cyclohexanedicarboxylic acid anhydride, isatoic acid anhydride, diphenic acid anhydride, substituted anhydrides, particularly including lower-alkyl substituted acid anhydrides such as butylsuccinic acid anhydride, hexylsuccinic acid anhydride, octylsuccinic acid anhydride, butylmaleic acid anhydride, pentylmaleic acid anhydride, hexylmaleic acid anhydride, octylmaleic acid anhydride, butylglutaric acid anhydride, hexylglutaric acid anhydride, heptylglutaric acid anhydride, octylglutaric acid anhydride, alkylcyclohexanedicarboxylic acid anhydrides and alkylphthalic acid anhydrides such as 4-n-butylphthalic acid anhydride, hexylphthalic acid anhydride, and octylphthalic acid anhydride.

In one particular embodiment, the carboxylic acid anhydride comprises hexahydrophthalic anhydride. Hexahydrophthalic anhydride may in some cases be the only carboxylic acid anhydride used in the reaction of step (b2).

The reaction of step (b2) provides a second intermediate product with a carboxylic acid group for each molecule of cyclic carboxylic acid anhydride reacted with the hydroxyl-functional first intermediate product of step (a). In some example embodiments, the equivalent ratio of the cyclic carboxylic acid anhydride to the first intermediate product is from about 0.8 to about 1.0, preferably from about 0.85 to about 1.0, and more preferably from about 0.9 to about 1.0 equivalents of anhydride groups per equivalent of hydroxyl groups. In one example embodiment, one molecule or substantially one molecule of anhydride reacts with each hydroxyl group of the first intermediate product to form the second intermediate product. In preferred embodiments, substantially all hydroxyl groups of the hydroxyl-functional first intermediate product are reacted with the carboxylic acid anhydride to provide an ester of the hydroxyl group and a carboxylic acid group from opening the cyclic anhydride.

The anhydride ring-opening reaction of step (b2) is exothermic. The reaction temperature can be controlled, for example to not exceed about 150° C., by dividing carboxylic acid anhydride reactant addition into two or more added portions. For example, a first added portion may be about one-third to about one-half of the carboxylic acid anhydride and a second portion may be the balance of the carboxylic acid anhydride being reacted in step (b2). The temperature of the reaction mixture may be allowed to cool to about 85° C. to 105° C. before each portion is added. After the first portion is added, the reaction mixture may be heated to about 110° C. to 115° C., or higher, resulting in an exotherm that may be allowed to carry the temperature of the reaction mixture upward, but not to exceed the target maximum, for example 150° C. After the exotherm, the reaction mixture may be cooled to about 85° C. to 105° C. for a second anhydride addition. Similarly, after the second anhydride addition has been completed, the reaction mixture may be heated to about 110° C. to 115° C., or higher, after which the reaction exotherm, (and additional heat, if needed), are used to bring the temperature of the reaction mixture up to, for example from about 135° C. to about 145° C. or from about 140° C. to about 145° C., where the reaction mixture is held to allow the reaction to complete.

In certain embodiments, the at least one carboxylic acid functional group of the second intermediate product of step (b2) is reacted with an epoxide-functional compound having one epoxide group to form the aryl-modified hyperbranched polyol. For instance, in step (c1), essentially all of the carboxylic acid functional groups of the second intermediate product are reacted with the epoxide-functional compound to form an aryl-modified hyperbranched polyol comprising hydroxyl functional groups, which is suitable for solventborne compositions. Accordingly, in step (c1), the equivalent ratio of carboxylic acid groups of the second intermediate product to epoxide groups of the epoxide-functional compound is preferably from about 1.0 to about 1.1. In a variant, only a portion of the carboxylic acid functional groups of the second intermediate product are reacted with the epoxide-functional compound to form an aryl-modified hyperbranched polyol comprising hydroxyl functional groups, which is suitable for waterborne compositions. For the waterborne coating compositions obtained in step (c2), the equivalent ratio carboxylic acid groups of the second intermediate product to epoxide groups of the epoxide-functional compound is about 1.75 to about 2.25 such that unreacted carboxylic acid groups remain.

Mono-epoxide compounds are well-known in the art, and may be characterized by general formula (VI):

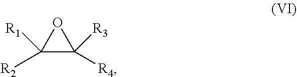

where R₁, R₂, R₃ and R₄ are each independently hydrogen or an organic radical, with the proviso that at least one of R₁-R₄ is other than hydrogen, and may contain unsaturation or heteroatoms or two of R₁-R₄ may form a cyclic ring, which may contain unsaturation or heteroatoms.

For example, the epoxide-functional compound of steps (c1) and (c2) may be an epoxy ester, also known as a glycidyl ester. Glycidyl esters can be prepared by reacting a monofunctional carboxylic acid with an epihalohydrin (e.g., epichlorohydrin) under conditions well known in the art. Examples of glycidyl esters are glycidyl acetate, glycidyl propionate, glycidyl methyl maleate, glycidyl stearate, glycidyl benzoate, and glycidyl oleate. Among useful glycidyl esters are those having an alkyl group having from 7 to 17 carbon atoms. A particularly preferred glycidyl ester is a glycidyl ester of a saturated synthetic tertiary monocarboxylic acid having 9-11 carbon atoms. In a preferred embodiment, the monofunctional carboxylic acid used to produce the glycidyl esters is a neoalkanoic acid such as, without limitation, neodecanoic or neononanoic acid. Glycidyl esters of neoacids are commercially available, e.g., under the trademark Cardura® from Momentive Specialty Chemicals, Inc., Columbus, Ohio.

Another useful class of monoepoxides is glycidyl ethers. Glycidyl ethers can be prepared by the reaction of monofunctional alcohols (e.g., n-butanol, propanol, 2-ethylhexanol, dodecanol, phenol, cresol, cyclohexanol, benzyl alcohol) with an epihalohydrin (e.g., epichlorohydrin). Useful glycidyl ethers include methyl glycidyl ether, ethyl glycidyl ether, propyl glycidyl ether, butyl glycidyl ether, pentyl glycidyl ether, hexyl glycidyl ether, heptyl glycidyl ether, octyl glycidyl ether, nonyl glycidyl ether, decyl glycidyl ether, undecyl glycidyl ether, dodecyl glycidyl ether, tridecyl glycidyl ether, tetradecyl glycidyl ether, pentadecyl glycidyl ether, hexadecyl glycidyl ether, heptadecyl glycidyl ether, octadecyl glycidyl ether, nonadecyl glycidyl ether, eicosyl glycidyl ether, beneicosyl glycidyl ether, docosyl glycidyl ether, tricosyl glycidyl ether, tetracosyl glycidyl ether, pentacosyl glycidyl ether, decenyl glycidyl ether, undecenyl glycidyl ether, tetradecenyl glycidyl ether, hexadecenyl glycidyl ether, heptadecenyl glycidyl ether, octadecenyl glycidyl ether, nonadecenyl glycidyl ether, eicosenyl glycidyl ether, beneicosenyl glycidyl ether, docosenyl glycidyl ether, tricosenyl glycidyl ether, tetracosenyl glycidyl ether, and pentacosenyl glycidyl ether.

The equivalent ratio in steps (c1) and (c2) of carboxylic acid groups of the second intermediate product to epoxide groups of the epoxide-functional compound may vary from about 1.0 to about 2.5 depending on whether the embodiment will be for a solventborne or waterborne coating composition. For the solventborne coating compositions obtained in step (c1), the equivalent ratio of carboxylic acid groups of the second intermediate product of step (b2) to epoxide groups of the epoxide-functional compound is about 1.0 to about 1.1 such that every, or substantially every, carboxyl group of the second intermediate product of step (b2) is reacted with a monoepoxide compound, whereas the equivalent ratio of carboxylic acid groups of the second intermediate product of step (b2) to epoxide groups of the epoxide-functional compound is about 1.75 to about 2.25 for the waterborne coating compositions obtained in step (c2) is such that on average some of the carboxyl groups are left unreacted and may be neutralized, for example with ammonia, an amine, or another base in forming a water-reducible coating composition.

For waterborne compositions, hydroxyl groups of the aryl-modified hyperbranched polyol obtained in step (c2) may be further reacted with lactone(s) in step (c2a), to obtain an aryl-modified hyperbranched polyol comprising carboxylic acid functional groups and polylactone chain extensions for waterborne coating compositions. In this step, a ring-opening polymerization of the lactone monomers, first, onto the hydroxyl functionality on the aryl-modified hyperbranched polyol obtained in step (c2), and then repeatedly onto the hydroxyl functionality created with each ring-opening reaction results in poly-lactone stabilization chains being grown from each hydroxyl group available on the aryl-modified hyperbranched polyol from step (c2).

The lactones and the reaction conditions useful in step (c2a) are the same as those mentioned for step (b1). Useful catalysts include those mentioned above for the esterification step (a) or step (a₀).

In other embodiments, a different third step (c3) first reacts the at least one carboxylic acid group of the second intermediate product of step (b2) with a polyol comprising at least three hydroxyl groups, to obtain a third intermediate product comprising hydroxyl functional groups. The polyols useful in step (c3) are the same as those used in steps (a) and (a₀) and the esterification of step (c3) is performed using the same methods and conditions as the esterification step (a) or step (a₀).

In step (c3), the ratio of moles of the polyol to moles of the second intermediate is from about 2.0 to about 2.5 moles of the polyol per mole of second intermediate product. Preferably, an equimolar amount of polyol monomers are reacted with the available carboxylic acid functionality of the second intermediate product of step (b2), to provide the third intermediate product of step (c3). In a preferred embodiment, the second intermediate product has approximately two carboxylic acid functional groups that react with the polyol monomers, such that each carboxylic functional group of the second intermediate product yields 2 to 7 hydroxyl functional groups in the third intermediate product. For instance, when the second intermediate product has two carboxylic functional groups, the third intermediate product has from 4 hydroxyl functional groups when the polyol is a triol (i.e. , 2 moles of triol with 2 carboxylic acid functional groups, yields 4 hydroxy functional groups) to 14 hydroxyl functional groups when the polyol is an octol. If necessary, the hydroxyl functionality may be increased by reacting the terminal hydroxyl groups with dimethylol propionic acid.

Thereafter, in a first variant denoted as step (d1), the hydroxyl functional groups of the third intermediate product of step (c3) are reacted with a lactone or a lactone mixture to grow poly-lactone stabilization chains. In step (d1), a ring-opening polymerization of the lactone monomers, first, onto the hydroxyl functionality on the third intermediate product, and then repeatedly onto the hydroxyl functionality created with each ring-opening reaction results in poly-lactone stabilization chains being grown from each hydroxyl group available on the third intermediate reaction product.

The lactones and the reaction conditions useful in step (d1) are the same as those mentioned for step (b1) and useful catalysts include those mentioned above for the esterification step (a) or step (a₀). It is sometimes preferred to utilize a blend of more than one type of lactone monomer when appending the poly-lactone stabilization chains in step (d1). This is to interrupt the crystallinity that can occur with long poly-lactone chains. For example, certain embodiments of step (d1) employ a mixture of c-caprolactone with δ-calerolactone.

The number of moles of lactone relative to the moles of the third intermediate product in step (d1) will determine the degree of extension of the poly-lactone chains. In solventborne coating compositions, the poly-lactone stabilization chains extend into the continuous phase of the composition and sterically hinder the agglomeration of organic pigments, which, due to their structural similarity with the aryl groups of the aryl-modified hyperbranched polyol, are anchored to the surface of the organic pigments.

In a second variant denoted as step (d2), the hydroxy functional groups of the third intermediate product from step (c3) are partially reacted with a cyclic carboxylic acid anhydride to form a fourth intermediate product comprising at least one carboxylic acid functional group and at least one hydroxyl functional group. Accordingly, the equivalent ratio of the cyclic carboxylic acid anhydride to the hydroxy functional groups available on the third intermediate product is less than 1.0.

In a third variant denoted as step (d3), all or substantially all of the hydroxy functional groups of the third intermediate product from step (c3) are reacted with a cyclic carboxylic acid anhydride to form a fourth intermediate product comprising carboxylic acid functional groups. In this variant, substantially all hydroxyl groups of the hydroxyl-functional third intermediate product are reacted with the carboxylic acid anhydride to provide an ester of the hydroxyl group and a carboxylic acid group from opening the cyclic anhydride in the fourth intermediate. Accordingly, the equivalent ratio of the cyclic carboxylic acid anhydride to the hydroxy functional groups available on the third intermediate product is usually about 1.0.

The cyclic carboxylic acid anhydride reacted in steps (d2) and (d3) may be either an aromatic or aliphatic cyclic anhydride and are the same as those described for use is step (b2).

In certain embodiments, the fourth intermediate comprising carboxylic acid functional groups and hydroxyl groups obtained from step (d2) may be reacted with lactone(s) in step (d2a). The hydroxyl functional groups of the fourth intermediate product of step (c3) are reacted with a lactone or a lactone mixture to grow poly-lactone stabilization chains. In step (d2a), a ring-opening polymerization of the lactone monomers, first, onto the hydroxyl functionality on the fourth intermediate product, and then repeatedly onto the hydroxyl functionality created with each ring-opening reaction results in poly-lactone stabilization chains being grown from each hydroxyl group available on the fourth intermediate reaction product. The lactones and the reaction conditions useful in step (d2a) are the same as those mentioned for steps (b1), (c2a), and (d1).

In a further step (e), the at least one carboxylic acid functional group of the fourth intermediate product of step (d3) is reacted with an epoxide-functional compound having one epoxide group to form the aryl-modified hyperbranched polyol. The equivalent ratio in step (e) of carboxylic acid groups of the fourth intermediate product to epoxide groups of the epoxide-functional compound may be from about 1.0 to about 2.5. The preferred range of equivalents of carboxylic acid groups to epoxide groups will vary, however, depending on whether the embodiment will be for a solventborne or waterborne coating composition.

For instance, in step (e1), every, or substantially every, carboxylic acid functional groups of the fourth intermediate product from step (d3) are reacted with the epoxide-functional compound to form an aryl-modified hyperbranched polyol comprising hydroxyl functional groups, which is suitable for solventborne compositions. Accordingly, in step (e1), the equivalent ratio of carboxylic acid groups of the second intermediate product to epoxide groups of the epoxide-functional compound is preferably from about 1.0 to about 1.1.

Alternatively, in step (e2), only a portion of the carboxylic acid functional groups of the fourth intermediate product of step (d3) are reacted with the epoxide-functional compound to form an aryl-modified hyperbranched polyol comprising hydroxyl functional groups, which is suitable for waterborne compositions. For the waterborne coating compositions obtained in step (e2), the equivalent ratio carboxylic acid groups of the fourth intermediate product to epoxide groups of the epoxide-functional compound is about 1.75 to about 2.25 equivalents such that unreacted carboxylic acid groups remain. The unreacted carboxyl groups may be neutralized, for example with ammonia, an amine, or another base in forming a water-reducible coating composition.

The same considerations discussed earlier for steps (c1) and (c2) concerning the selection and reaction of epoxide functional compounds apply to their use in steps (e1) and (e2), and the mono-epoxide compounds useful for steps (e1) and (e2) are the same as those employed for steps (c1) and (c2).

Afterward, for some embodiments, the hydroxyl functional groups of the aryl-modified hyperbranched polyol prepared in steps (e1) and (e2) are reacted in steps (el a) and (e2a), respectively, with a lactone or lactone mixture to append polylactone chains which extend from the aryl-modified hyperbranched polyol to produce an aryl-modified hyperbranched polyol with polylactone chain extensions. The lactones and the reaction conditions useful in steps (e1a) and (e2a) are the same as those mentioned for steps (b1), (d1), (c2a), and (d2a). The aryl-modified hyperbranched polyol with polylactone chain extensions obtained in step (e2a) is suitable for solventborne compositions, while the aryl-modified hyperbranched polyol with polylactone chain extensions additionally includes carboxylic acid functional groups, and this, is useful for waterborne compositions.

### GENERAL EMBODIMENTS

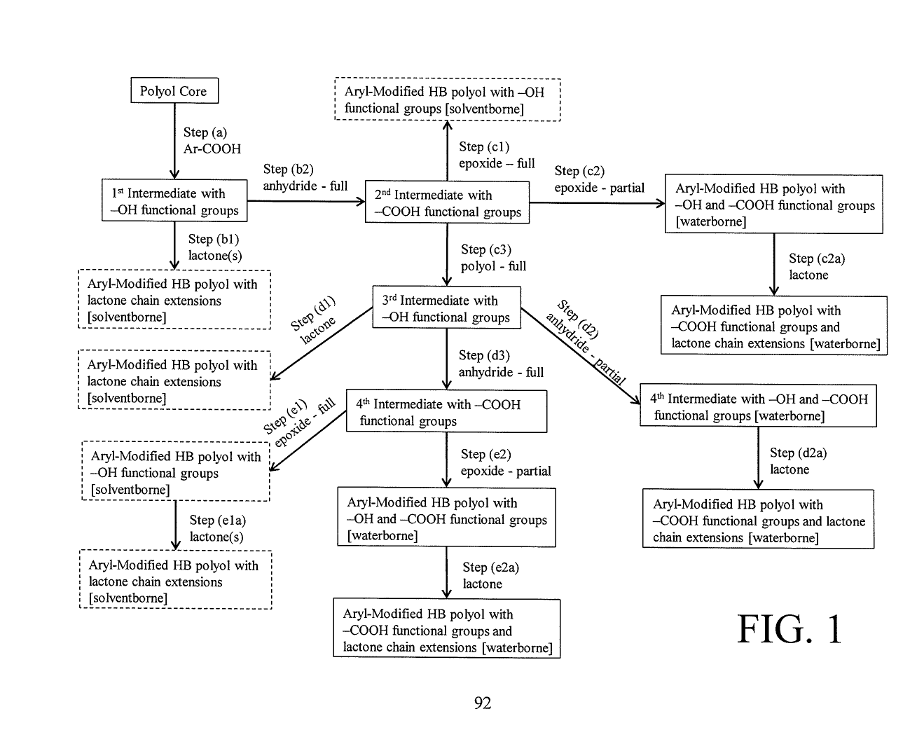

The reaction or step numbers mentioned hereafter refer to reaction descriptions as they are presented in the Detailed Disclosure section of the application and shown in FIG. 1.

### General Embodiment 1. An Aryl-Modified Hyperbranched Polyol for Waterborne Systems

This aryl-modified hyperbranched polyol embodiment obtained from reactions (c2) and (c3) is suitable for use with water sensitive pigments used in waterborne coating compositions, and includes:
a) a core comprising hydroxyl functional groups, where the core is selected from the group consisting of: a polyol comprising at least three hydroxyl functional groups; a polyester polyol comprising, in reacted form, a polyol comprising at least three hydroxyl groups and at least one selected from group consisting of an aliphatic dicarboxylic acid having from 6 to 36 carbon atoms and an aromatic dicarboxylic acid having from 8 to 20 carbon atoms; and a polyurethane polyester polyol comprising, in reacted form, a polyol comprising at least three hydroxyl groups and a polyurethane diacid according to the following formula:

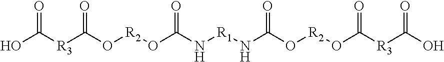

wherein:
R₁ and R₂ are each a C₂-C₁₆ aliphatic group, preferably a C₃-C₈ aliphatic group, and especially a C₃-C₆ aliphatic group; and
R₃ is a C₂-C₂₂ substituted or unsubstituted aliphatic, alicyclic, or aromatic group, preferably a C₆-C₁₈ alicyclic, or aromatic group;
b) an aryl-modifier attached to the polyester polyol core, the aryl-modifier being formed from an aromatic carboxylic acid of formula (I):

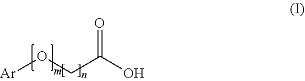

wherein: Ar is an optionally substituted aryl group, which is a monocyclic or condensed polycyclic aromatic hydrocarbon group; m is 0 or 1; and n is 0 or an integer from 1 to 8;
c) an intermediate substituent further attached to the polyester polyol core, the intermediate substituent being formed from a compound selected from the group consisting of polyfunctional carboxylic anhydrides and acids thereof;
d) a chain extension attached to the intermediate substituent, wherein the chain extension comprises a hydroxyl group and is formed from a compound having a terminal or non-terminal epoxide group thereon; and
e) optionally, a terminal polylactone chain extension that is attached the chain extension.

The water-reducible aryl-modified hyperbranched formed from a polyol compound have the following formula:

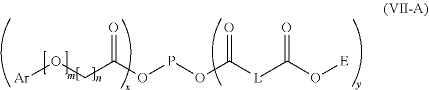

wherein:
P is a C₃-C₂₀ polyol moiety bonded to x groups of formula (I) and y groups of formula —C(O)—L—C(O)—O—E through x+y —OH functionalities;
x is an integer from 1 to 7, preferably from 4 to 6;
y is an integer from 2 to 7, preferably from 2 to 4;
with the proviso that the sum of x +y is from 3 to 8;
each L is independently a C₂-C₂₂ substituted or unsubstituted aliphatic, alicyclic, or aromatic moiety;
E is hydrogen or —C(OH)R₅, where R₅ is a substituted or unsubstituted C₁-C₂₂ aliphatic, alicyclic, or aromatic moiety, with the proviso that at least one E is —C(OH)R₅ and at least one E is hydrogen;
each Ar is an optionally substituted aryl group, which is a monocyclic or condensed polycyclic aromatic hydrocarbon group;
each m is 0 or 1; and
each n is 0 or an integer from 1 to 8, preferably 0 or an integer of 1 to 3.

Optionally, any E groups of formula —C(OH)R₅ may be capped with a terminal lactone chain via the OH group.

The water-reducible aryl-modified hyperbranched formed from a hydroxy-functional core based on a polyol and a dicarboxylic acid or polyurethane diacid have the following formula:

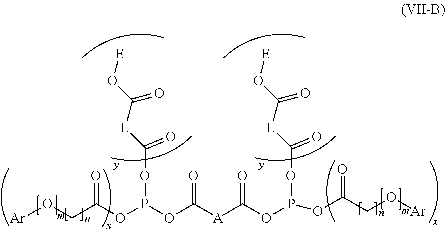

wherein:
A is C₄-C₃₄ aliphatic moiety, a C₆-C₁₈ aromatic moiety, or a moiety having the following formula:

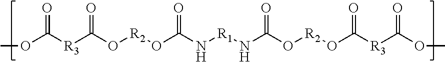

wherein:
R₁ and R₂ are each a C₃-C₈ aliphatic group; and
R₃ is a C₆-C₁₈ substituted or unsubstituted aliphatic, alicyclic, or aromatic group;
each P is independently a C₃-C₂₀ polyol moiety bonded to x groups of formula (I) and y groups of formula —C(O)—L—C(O)—O—E through x+y —OH functionalities;
each x is an integer from 1 to 6, preferably from 1 to 3;
each y is an integer from 1 to 6, preferably from 1 to 2;
with the proviso that the sum of x +y on each polyol P is from 2 to 7;
each L is independently a C₂-C₂₂ substituted or unsubstituted aliphatic, alicyclic, or aromatic moiety;
each E is hydrogen or —C(OH)R₅, where R₅ is a substituted or unsubstituted C₁-C₂₂ aliphatic, alicyclic, or aromatic moiety, with the proviso that at least one E is —C(OH)R₅ and at least one E is hydrogen;
each Ar is an optionally substituted aryl group, which is a monocyclic or condensed polycyclic aromatic hydrocarbon group;
each m is 0 or 1; and
each n is 0 or an integer from 1 to 8, preferably 0 or an integer of 1 to 3.

Optionally, any E groups of formula —C(OH)R₅ may be capped with a terminal lactone chain via the OH group.

In one embodiment, the aryl-modified hyperbranched polyester has formula (VIII-A):

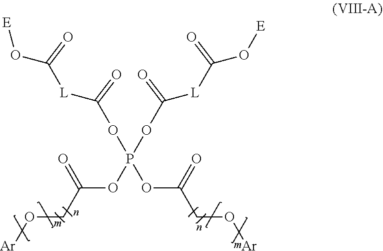

wherein:
P is a tetrol;
each L is independently a C₂-C₂₂ substituted or unsubstituted aliphatic, alicyclic, or aromatic moiety;
each E is hydrogen or —C(OH)R₅, where R₅ is a substituted or unsubstituted C₁-C₂₂ aliphatic, alicyclic, or aromatic moiety, with the proviso that at least one E is hydrogen and at least one E is —C(OH)R₅;
each Ar is an optionally substituted phenyl or naphthyl group;
each m is 0 or 1; and
each n is 0 or an integer of 1 to 3.

Optionally, the E group of formula —C(OH)R₅ may be capped with a terminal lactone chain via the OH group.

In another embodiment, the aryl-modified hyperbranched polyester has formula (VIII-B):

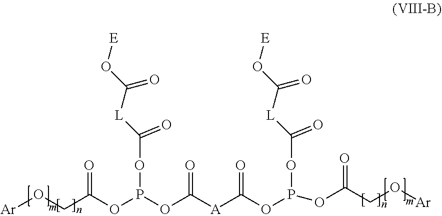

wherein:
A is C₄-C₃₄ aliphatic moiety, a C₆-C₁₂ aromatic moiety, or a moiety having the following formula:

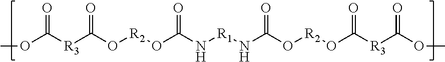

wherein:
R₁ and R₂ are each a C₃-C₈ aliphatic group; and
R₃ is a C₆-C₁₈ substituted or unsubstituted aliphatic, alicyclic, or aromatic group;
each P is a triol, such as trimethylolmethane, trimethylolethane, trimethylolpropane, bonded through its —OH functionalities;
each L is independently a C₂-C₂₂ substituted or unsubstituted aliphatic, alicyclic, or aromatic moiety;
each E is hydrogen or —C(OH)R₅, where R₅ is a substituted or unsubstituted C₁-C₂₂ aliphatic, alicyclic, or aromatic moiety, with the proviso that at least one E is hydrogen and at least one E is —C(OH)R₅;
each Ar is an optionally substituted phenyl or naphthyl group;
each m is 0 or 1; and
each n is 0 or an integer of 1 to 3.

Optionally, the E group of formula —C(OH)R₅ may be capped with a terminal lactone chain via the OH group.

A preferred compound of formula (VIII-A) and formula (VIII-B) are shown below:

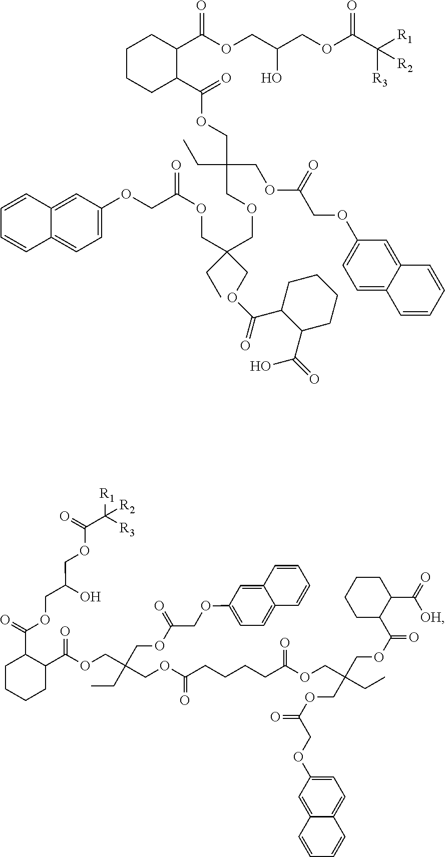

wherein R₁R₂R₃ is neodecanoate.

In another embodiment, the aryl-modified hyperbranched polyester has formula (IX):

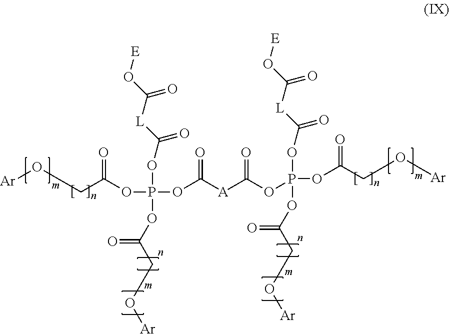

wherein:
A is C₄-C₁₀ aliphatic moiety, a C₆-C₁₂ aromatic moiety, or a moiety having the following formula:

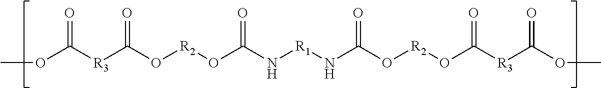

wherein:
R₁ and R₂ are each a C₃-C₈ aliphatic group; and
R₃ is a C₆-C₁₈ substituted or unsubstituted aliphatic, alicyclic, or aromatic group;
each P is a tetrol, such as di(trimethylolpropane), bonded through its —OH functionalities;
each L is independently a C₂-C₂₂ substituted or unsubstituted aliphatic, alicyclic, or aromatic moiety;
each E is hydrogen or —C(OH)R₅, where R₅ is a substituted or unsubstituted C₁-C₂₂ aliphatic, alicyclic, or aromatic moiety, with the proviso that at least one E is —C(OH)R₅ and at least one E is hydrogen, preferably one E is —C(OH)R₅ and one E is hydrogen;
each Ar is an optionally substituted phenyl or naphthyl group;
each m is 0 or 1; and
each n is 0 or an integer of 1 to 3.

Optionally, the E group of formula —C(OH)R₅ may be capped with a terminal lactone chain via the OH group.

A particularly preferred aryl-modified hyperbranched polyester has formula (X):

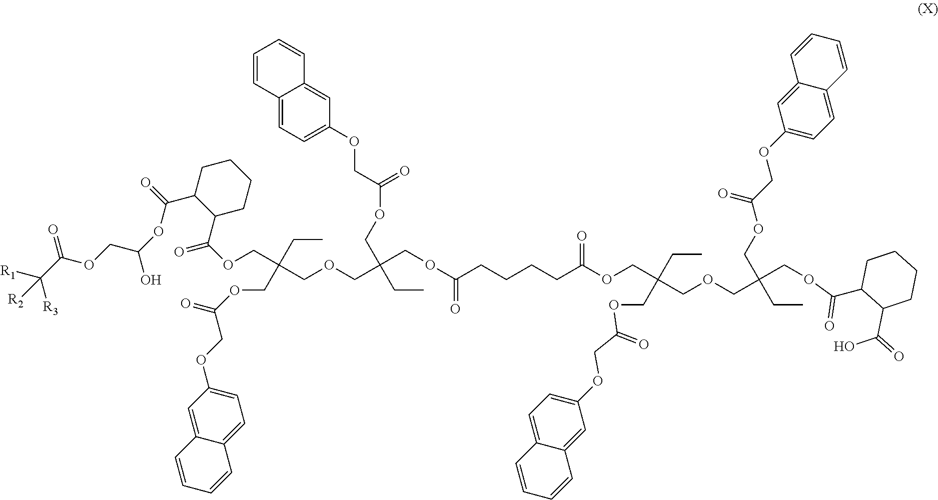

wherein R₁R₂R₃ is neodecanoate.

As described above, one method for preparing aryl-modified hyperbranched polyols of formula (VII-B) includes a first step of reacting a polyol comprising at least three hydroxyl groups with an aliphatic dicarboxylic acid having from 6 to 36 carbon atoms, or an aromatic dicarboxylic acid having from 8 to 20 carbon atoms, (or an esterifiable derivative thereof), to form a core comprising hydroxyl functional groups, which, for illustration, is shown below in reference to the preparation of the aryl-modified hyperbranched polyol of formula (X):

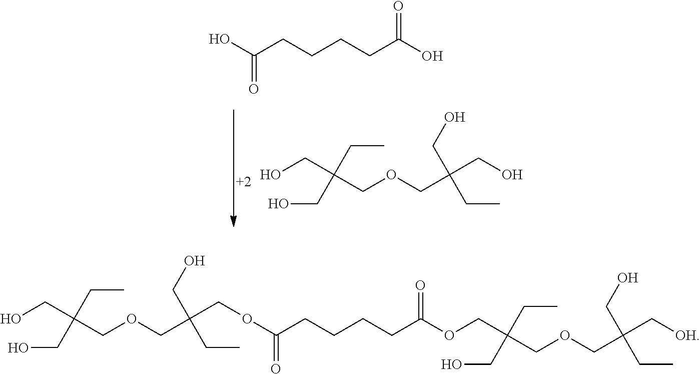 [SMILES: O=C(O)CCCCC(=O)O ; CCC(CO)(CO)COCC(CC)(CO)CO ; CCC(CO)(CO)COCC(CC)(CO)COC(=O)CCCCC(=O)OCC(CC)(CO)COCC(CC)(CO)CO]

After forming the polyester polyol core, the core is reacted with an aromatic carboxylic acid of the formula (I), to obtain a first intermediate product comprising hydroxyl functional groups, as shown below:

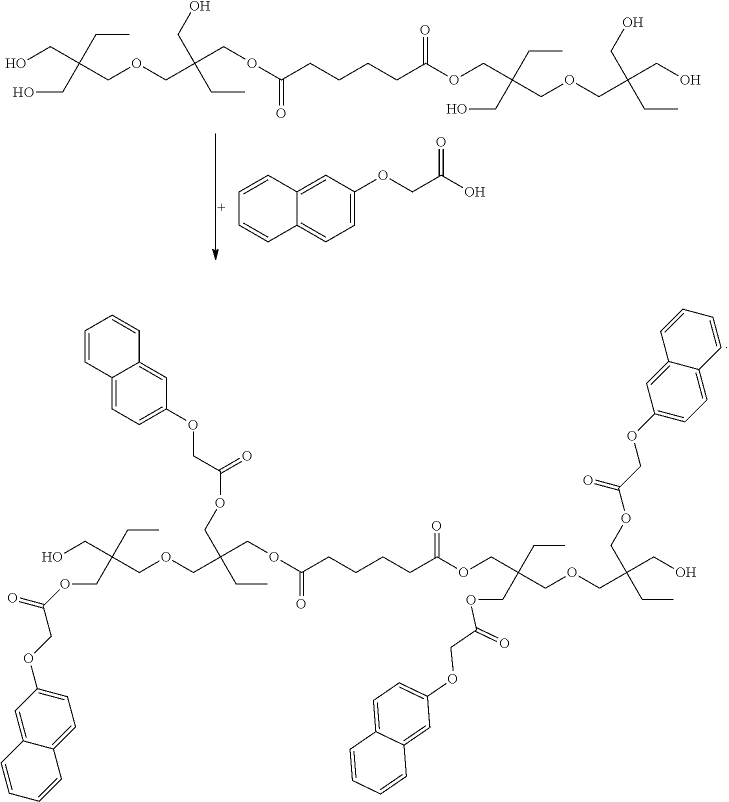

Here, —OH functionalities of the polyester polyol core remain in the first intermediate product. Therefore, after the addition of the aryl groups to the polyester polyol core, remaining —OH functionalities are reacted with a cyclic carboxylic acid anhydride, to form a carboxylic acid-functional second intermediate product comprising carboxylic acid functional groups, as shown below:

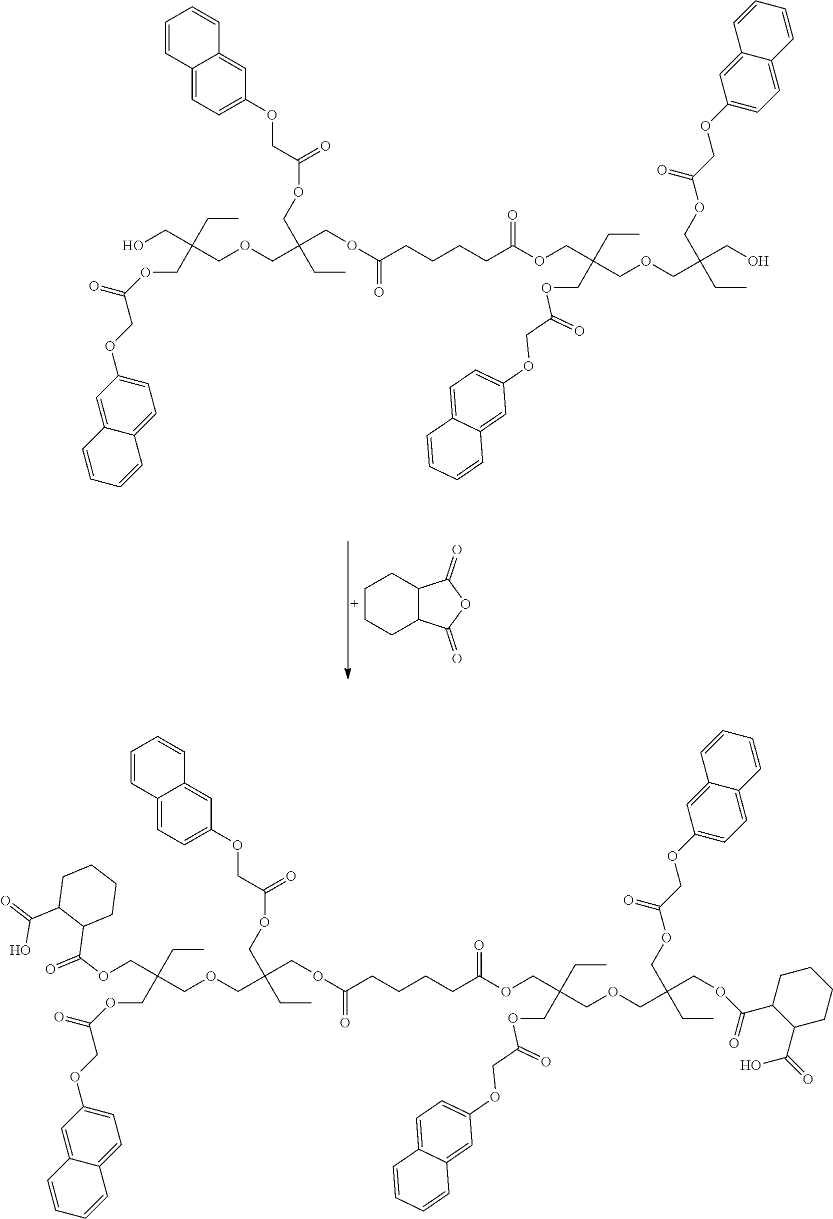 [SMILES: CCC(CO)(COCC(CC)(COC(=O)CCCCC(=O)OCC(CC)(COCC(CC)(CO)COC(=O)COc1ccc2ccccc2c1)COC(=O)COc1ccc2ccccc2c1)COC(=O)COc1ccc2ccccc2c1)COC(=O)COc1ccc2ccccc2c1 ; O=C1OC(=O)C2CCCCC12 ; CCC(COCC(CC)(COC(=O)COc1ccc2ccccc2c1)COC(=O)C1CCCCC1C(=O)O)(COC(=O)CCCCC(=O)OCC(CC)(COCC(CC)(COC(=O)COc1ccc2ccccc2c1)COC(=O)C1CCCCC1C(=O)O)COC(=O)COc1ccc2ccccc2c1)COC(=O)COc1ccc2ccccc2c1]

Finally, the carboxylic acid groups of the carboxylic acid-functional second intermediate are reacted with an epoxide-functional compound having one epoxide group, to form the aryl-modified hyperbranched polyol:

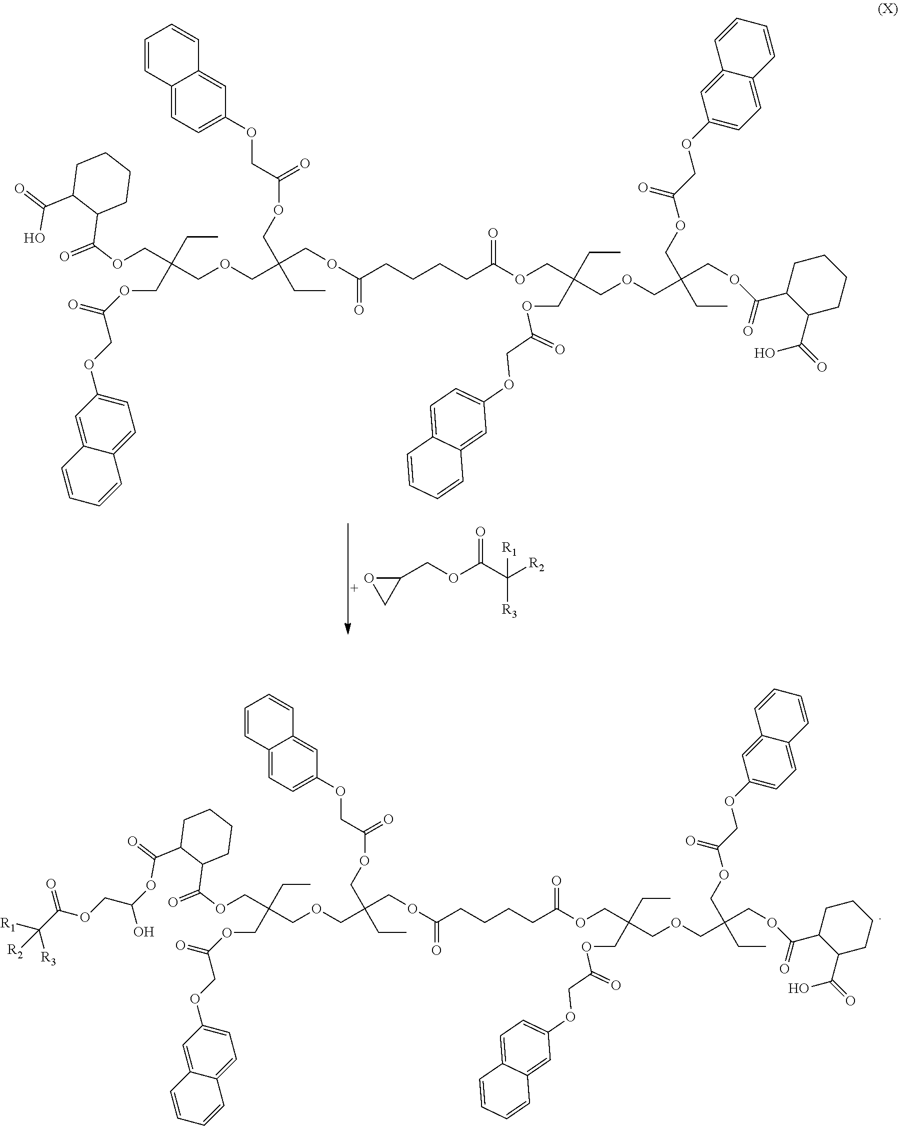

On average, some of the carboxyl groups are left unreacted, as shown above, and may be neutralized, for example with ammonia, an amine, or another base in forming a waterborne coating composition.

Additionally, any hydroxyl groups remaining on the aryl-modified hyperbranched polyols may be reacted with lactone(s) to add polylactone chain extensions.

### GENERAL EMBODIMENT 2. An aryl-modified hyperbranched polyol with polylactone stabilization chains for solventborne systems

This aryl-modified hyperbranched polyol embodiments obtained from reactions (b1), (d1), and (e1a) are suitable for stabilizing organic pigments in solventborne coating compositions, and include:
a) a core comprising hydroxyl functional groups, where the core is selected from the group consisting of a polyol comprising at least three hydroxyl functional groups; a polyester polyol comprising, in reacted form, a polyol comprising at least three hydroxyl groups and at least one selected from group consisting of an aliphatic dicarboxylic acid having from 6 to 36 carbon atoms and an aromatic dicarboxylic acid having from 8 to 20 carbon atoms; and a polyurethane polyester polyol comprising, in reacted form, a polyol comprising at least three hydroxyl groups and a polyurethane diacid according to the following formula:

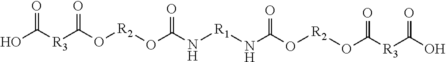

wherein:
R₁ and R₂ are each a C₂-C₁₆ aliphatic group, preferably a C₃-C₈ aliphatic group, and especially a C₃-C₆ aliphatic group; and
R₃ is a C₂-C₂₂ substituted or unsubstituted aliphatic, alicyclic, or aromatic group, preferably a C₆-C₁₈ alicyclic, or aromatic group;
b) an aryl-modifier attached to the polyester polyol core, the aryl-modifier being formed from an aromatic carboxylic acid of formula (I):

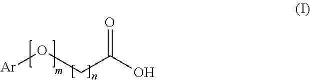

wherein Ar is an optionally substituted aryl group, which is a monocyclic or condensed polycyclic aromatic hydrocarbon group; m is 0 or 1; and n is 0 or an integer from 1 to 8;
c) optionally, a first linking moiety comprising a first intermediate substituent further attached to the polyester polyol core and a second intermediate substituent attached to the first intermediate substituent of the first linking moiety, the first intermediate substituent of the first linking moiety being formed from a compound selected from the group consisting of polyfunctional carboxylic anhydrides and acids thereof, and the second intermediate substituent of the first linking moiety being formed from a polyol having at least three hydroxyl groups;
d) optionally, a second linking moiety comprising a first intermediate substituent further attached to the polyester polyol core and a second intermediate substituent attached to the first intermediate substituent of the second linking moiety or, if the first linking moiety is present, attached to the second intermediate substituent of the first linking moiety, the first intermediate substituent of the second linking moiety being formed from a compound selected from the group consisting of polyfunctional carboxylic anhydrides and acids thereof, and the second intermediate substituent of the second linking moiety comprising a hydroxyl group and formed from a compound having a terminal or non-terminal epoxide group thereon; and
e) a poly-lactone chain moiety chain attached to the polyol core via the hydroxyl groups of the polyol core, or attached to the second intermediate substituent of the first linking moiety via the hydroxyl groups of the polyol if the first linking moiety is present and the second linking moiety is absent, or attached to the second intermediate substituent of the second linking moiety via the hydroxyl group(s) of the second linking moiety if the second linking moiety is present.

In certain embodiments, the aryl-modified hyperbranched polyester polyols have the following formula:

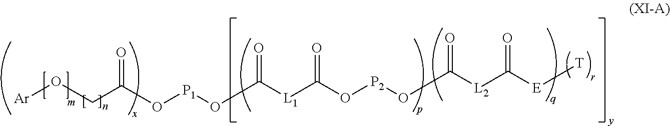

wherein:
P₁ is a C₃-C₂₀ polyol moiety bonded to x groups of formula (I) and y groups of formula —{C(O)—L₁—C(O)—O—P₂—O}ₚ—{C(O)—L₂—C(O)—E}_{q}—(T)ᵣ through x+y —OH functionalities;
x is an integer from 1 to 8, preferably from 2 to 6, more preferably 4 to 6;
y is an integer from 1 to 8, preferably 1 to 2, more preferably 2;
with the proviso that the sum of x+y is from 3 to 10;
each L₁ and each L₂ is independently a C₂-C₂₂ substituted or unsubstituted aliphatic, alicyclic, or aromatic moiety;
each P₂ is independently a C₃-C₂₀ polyol moiety bonded to q groups of —{C(O)—L₂—C(O)—E} through q —OH functionalities or to r groups of T through r —OH functionalities;
each E is —C(R₅)—O— where R₅ is a substituted or unsubstituted C₁-C₂₂ aliphatic, alicyclic, or aromatic moiety;
p is 0 or 1;
when p is 0, q is 0 or 1 and r is 1;
when p is 1, q is 0 and r is an integer from 2 to 7 or q is an integer from 2 to 7 and r is 1;
each T is (poly)lactone chain moiety having from 1 to 30, preferably from 5 to 30, lactone units;
each Ar is an optionally substituted aryl group, which is a monocyclic or condensed polycyclic aromatic hydrocarbon group;
each m is 0 or 1; and
each n is 0 or an integer from 1 to 8, preferably 0 or an integer of 1 to 3.

In some embodiments, the aryl-modified hyperbranched polyester polyols have the following formula:

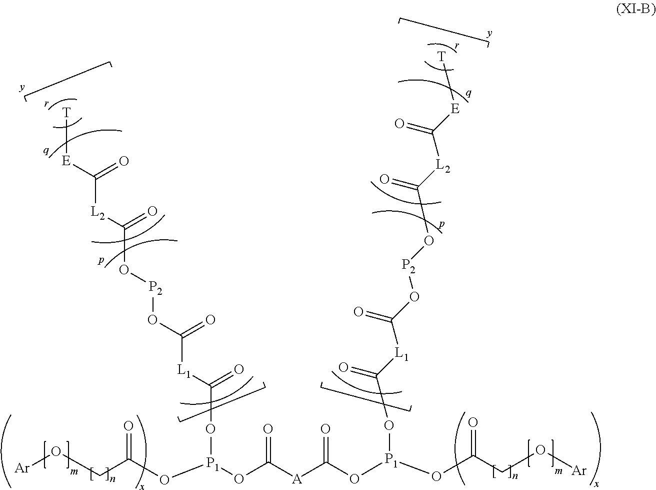

wherein:
A is C₄-C₃₄ aliphatic moiety, a C₆-C₁₈ aromatic moiety, or a moiety having the following formula:

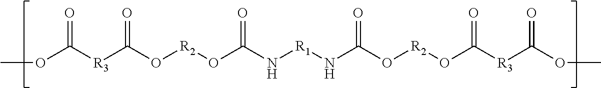

wherein:
R₁ and R₂ are each a C₃-C₈ aliphatic group; and
R₃ is a C₆-C₁₈ substituted or unsubstituted aliphatic, alicyclic, or aromatic group;
P₁ is a C₃-C₂₀ polyol moiety bonded to x groups of formula (I) and y groups of formula —{C(O)—L₁—C(O)—O—P₂—O}ₚ—{C(O)—L₂—C(O)—E}_{q}—(T)ᵣ through x+y —OH functionalities;
each x is an integer from 1 to 6, preferably from 2 to 6, more preferably 3 or 4;
each y is an integer from 1 to 6, preferably 1 to 2, more preferably 2;
with the proviso that the sum of x+y on each polyol P₁ is from 2 to 7;
each L₁ and each L₂ is independently a C₂-C₂₂ substituted or unsubstituted aliphatic, alicyclic, or aromatic moiety;
each P₂ is independently a C₃-C₂₀ polyol moiety bonded to q groups of —{C(O)—L₂—C(O)—E} through q —OH functionalities or to r groups of T through r —OH functionalities;
each E is —C(R₅)—O— where R₅ is a substituted or unsubstituted C₁-C₂₂ aliphatic, alicyclic, or aromatic moiety;
each p is 0 or 1;
when p is 0, q is 0 or 1 and r is 1;
when p is 1, q is 0 and r is an integer from 2 to 7 or q is an integer from 2 to 7 and r is 1;
each T is (poly)lactone chain moiety having from 1 to 30, preferably 5 to 30, lactone units;
each Ar is an optionally substituted aryl group, which is a monocyclic or condensed polycyclic aromatic hydrocarbon group;
each m is 0 or 1; and
each n is 0 or an integer from 1 to 8, preferably 0 or an integer of 1 to 3.

Preferably, the aryl-modified hyperbranched polyester has formula (XII-A), (XIII-A), (XIV-A), or (XV-A):

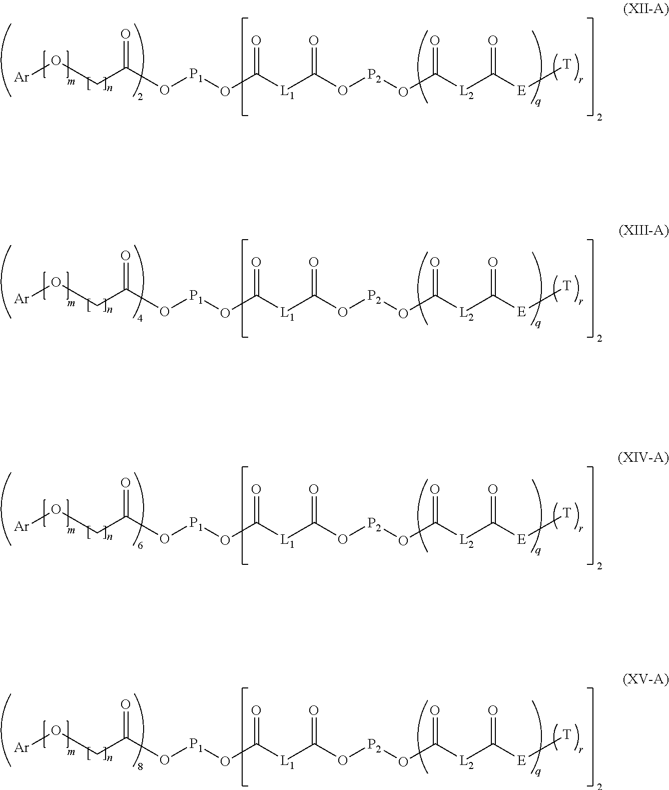

wherein:
each P₁ is a polyol having 4 —OH functionalities in formula (XII-A), 6 —OH functionalities in formulae (XIII-A), 8 —OH functionalities in formula (XIV-A), and 10 —OH functionalities in formula (XV-A);
each L₁ and each L₂ is independently a C₂-C₂₂ substituted or unsubstituted aliphatic, alicyclic, or aromatic moiety;
each P₂ is independently selected from the group of glycerol (q=2 and r=1, or q=0 and r=2), trimethylolpropane (q=2 and r=1, or q=0 and r=2), di(trimethylolpropane) (q=3 and r=1, or q=0 and r=3), pentaerythritol (q=3 and r=1, or q=0 r=3), di(pentaerythritol) (q=5 and r=1, or q=0 r=5), and tri(pentaerythritol) (q=7 and r=1, or q=0 r=7);
each E is —C(R₅)—O— where R₅ is a substituted or unsubstituted C₁-C₂₂ aliphatic, alicyclic, or aromatic moiety;
each T is (poly)lactone chain moiety having from 1 to 30, preferably 5 to 30, lactone units;
each Ar is an optionally substituted phenyl or naphthyl group;
each m is 0 or 1; and
each n is 0 or an integer of 1 to 3.

Preferably, the aryl-modified hyperbranched polyester has formula (XII-B), (XIII-B), (XIV-B), or (XV-B):

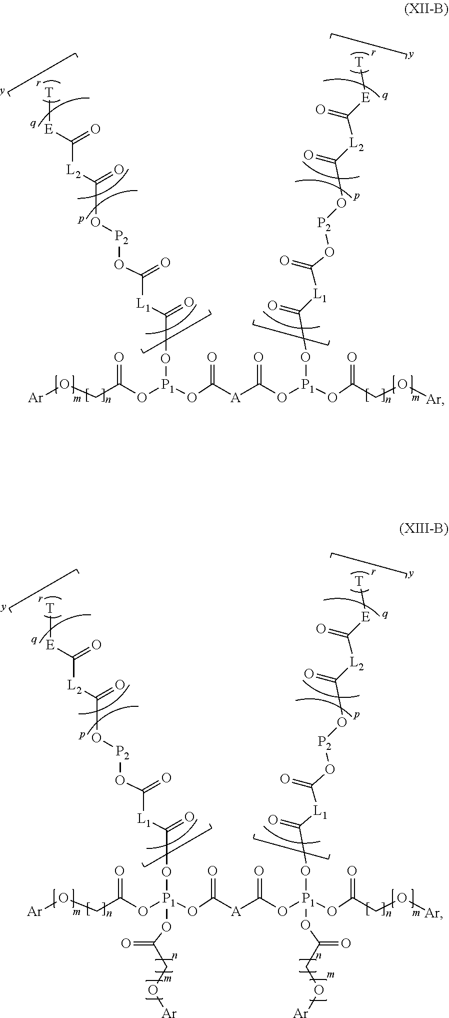

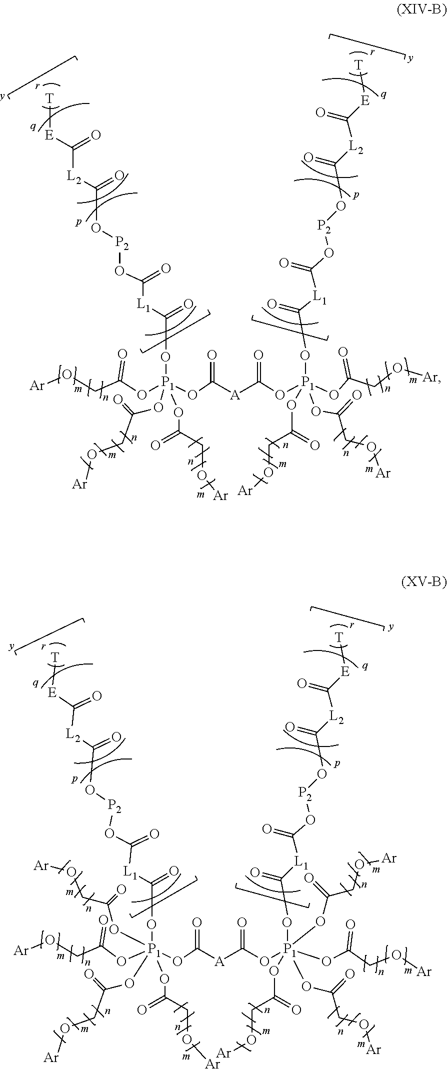

wherein:
A is C₄-C₁₀ aliphatic moiety, a C₆-C₁₂ aromatic moiety, or a moiety having the following formula:

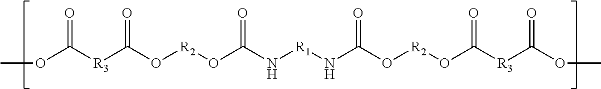

wherein:
R₁ and R₂ are each a C₃-C₈ aliphatic group; and
R₃ is a C₆-C₁₈ substituted or unsubstituted aliphatic, alicyclic, or aromatic group;
each P₁ is a polyol having 3 —OH functionalities in formula (XII-B); 4 —OH functionalities in formula (XIII-B), 5 —OH functionalities in formula (XIV-B), and 6 —OH functionalities in formula (XV-B);
each L₁ and each L₂ is independently a C₂-C₂₂ substituted or unsubstituted aliphatic, alicyclic, or aromatic moiety;
when p=1, each P₂ is independently selected from the group of glycerol (q=2 and r=1, or q=0 and r=2), trimethylolpropane (q=2 and r=1, or q=0 and r=2), di(trimethylolpropane) (q=3 and r=1, or q=0 and r=3), pentaerythritol (q=3 and r=1, or q=0 and r=3), di(pentaerythritol) (q=5 and r=1, or q=0 r=5), and tri(pentaerythritol) (q=7 and r=1, or q=0 r=7);
when p=0, q is 0 or 1 and r is 1;
each E is —C(R₅)—O— where R₅ is a substituted or unsubstituted C₁-C₂₂ aliphatic, alicyclic, or aromatic moiety;
each T is (poly)lactone chain moiety having from 1 to 30, preferably 5 to 30, lactone units;
each Ar is an optionally substituted phenyl or naphthyl group;
each m is 0 or 1; and
each n is 0 or an integer of 1 to 3.

Preferred compounds of formula (XI-B) have formula (XVI) and (XVII):

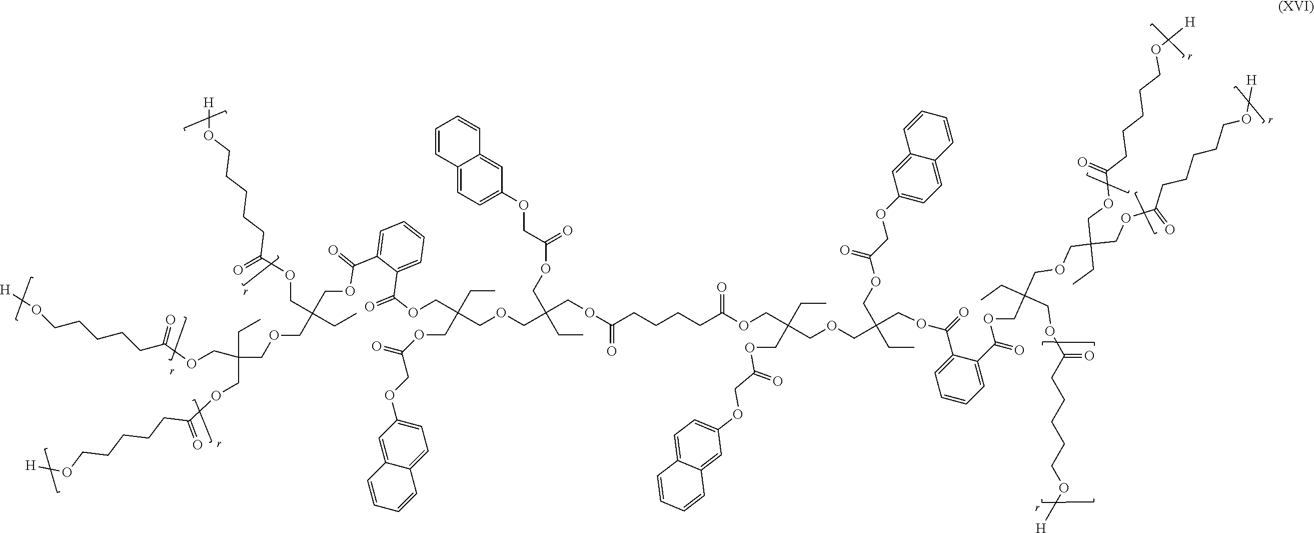

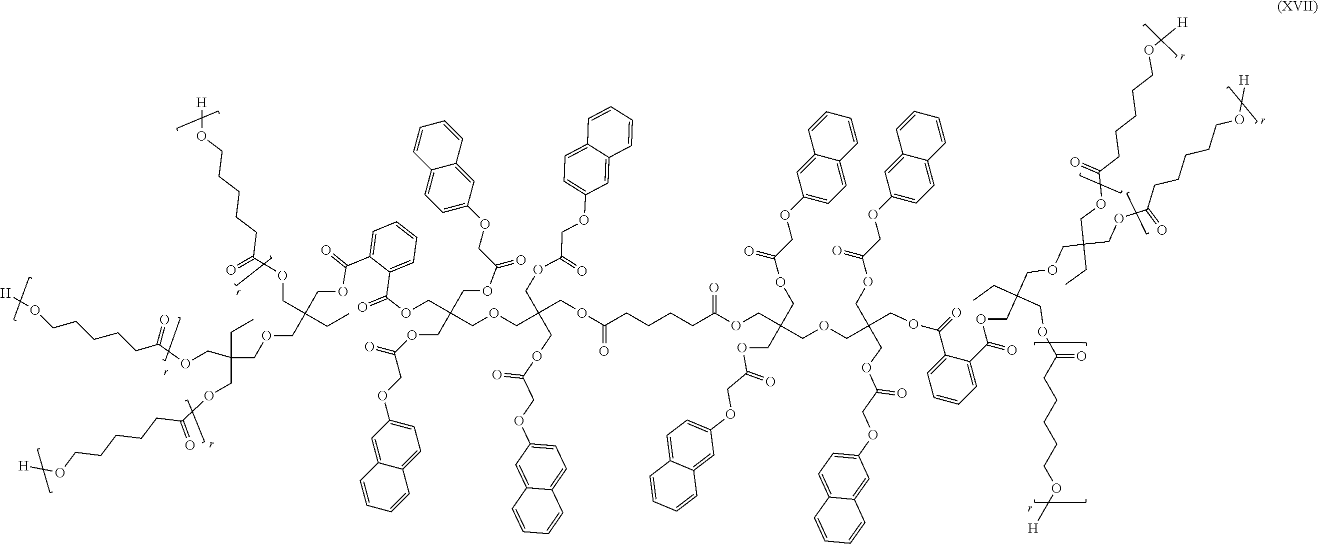

wherein r is from 1 to 30, preferably from 5 to 30.

The aryl-modified hyperbranched polyols according to formula (XI-B) are prepared in the same manner as aryl-modified hyperbranched polyols according to formula (X) except that the second intermediate is reacted with a polyol comprising at least three hydroxyl groups in step (c3) rather than an epoxide-functional compound having one epoxide group as described in steps (e1) and (c2), as shown below in reference to the preparation of the aryl-modified hyperbranched polyester polyol of formula (XVI):

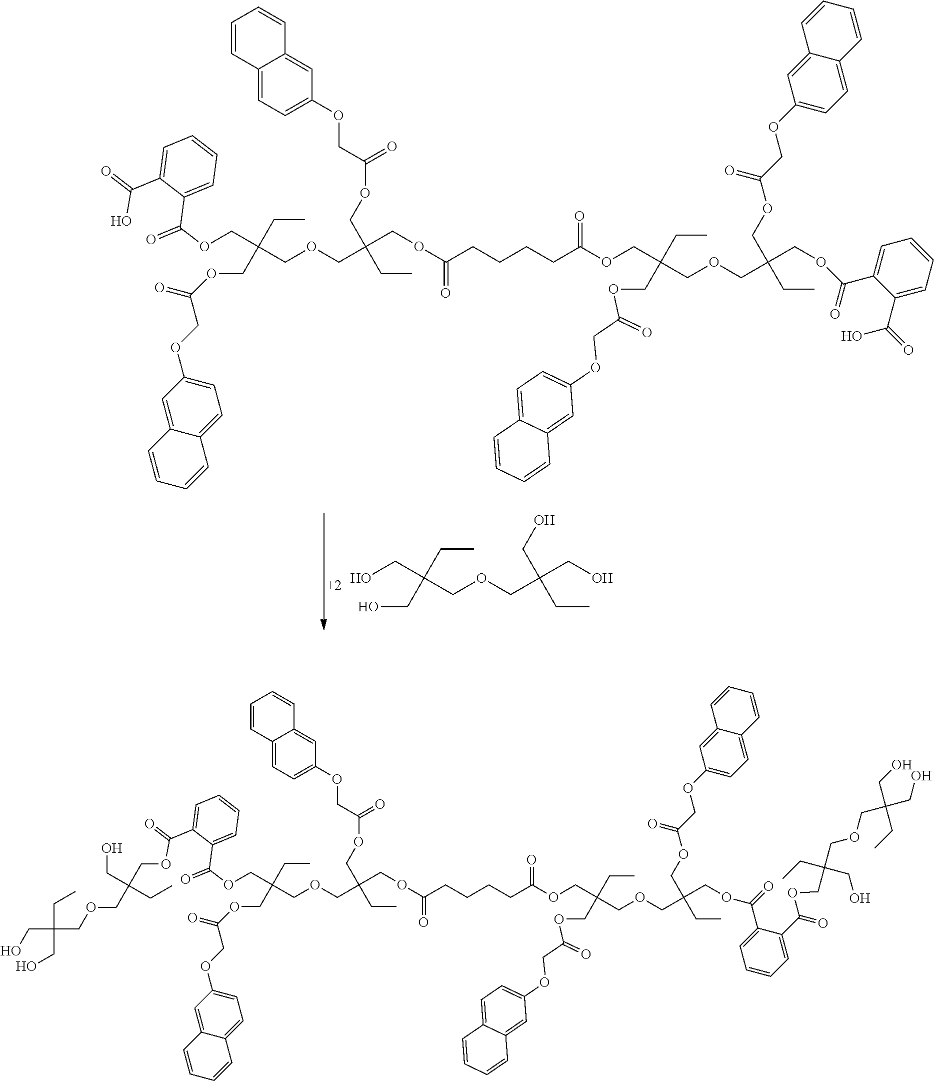 [SMILES: CCC(COCC(CC)(COC(=O)COc1ccc2ccccc2c1)COC(=O)c1ccccc1C(=O)O)(COC(=O)CCCCC(=O)OCC(CC)(COCC(CC)(COC(=O)COc1ccc2ccccc2c1)COC(=O)c1ccccc1C(=O)O)COC(=O)COc1ccc2ccccc2c1)COC(=O)COc1ccc2ccccc2c1 ; CCC(CO)(CO)COCC(CC)(CO)CO ; CCC(CO)(CO)COCC(CC)(CO)COC(=O)c1ccccc1C(=O)OCC(CC)(COCC(CC)(COC(=O)CCCCC(=O)OCC(CC)(COCC(CC)(COC(=O)COc1ccc2ccccc2c1)COC(=O)c1ccccc1C(=O)OCC(CC)(CO)COCC(CC)(CO)CO)COC(=O)COc1ccc2ccccc2c1)COC(=O)COc1ccc2ccccc2c1)COC(=O)COc1ccc2ccccc2c1]

After preparing the third intermediate product in step (c3), a ring-opening polymerization of the lactone monomers, first, onto the hydroxyl functionality on the third intermediate product, and then repeatedly onto the hydroxyl functionality created with each ring-opening in step (d1) yields the aryl-modified hyperbranched polyol with poly-lactone stabilization chains:

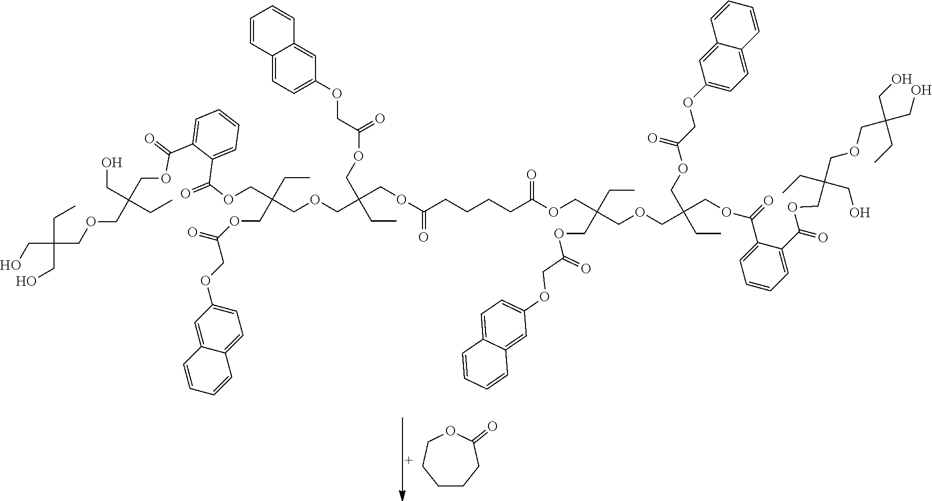

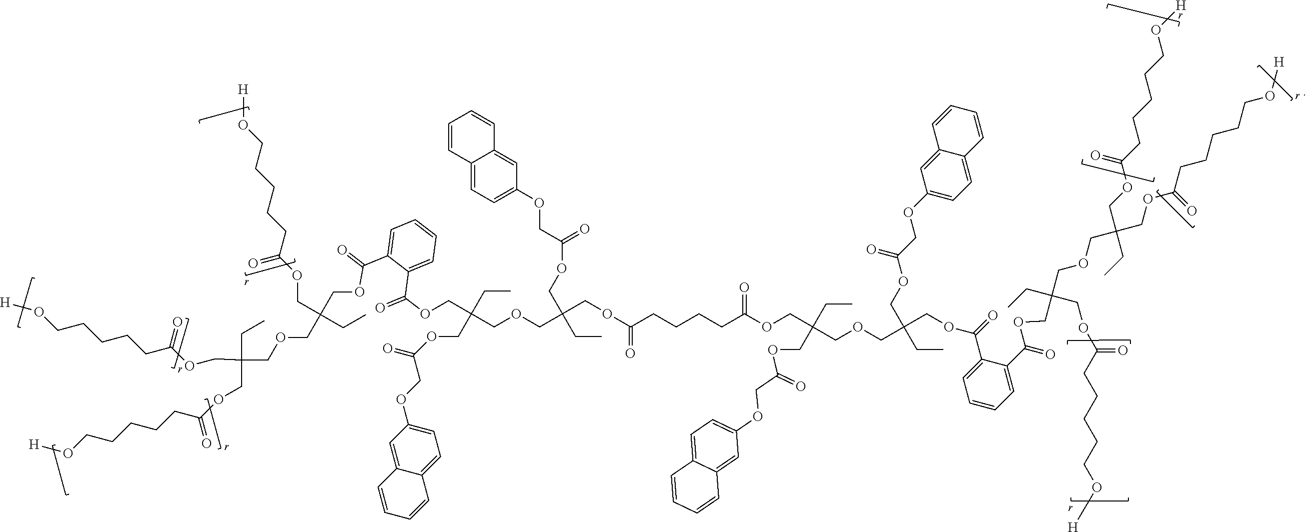

### Coating Compositions of the Aryl-Modified Hyperbranched Polyol

A desired amount of the aryl-modified hyperbranched polyol is included in the coating composition. The amount of the aryl-modified hyperbranched polyol included is not particularly limited and may vary depending on the characteristics of other coating components and the desired overall balance of performance characteristics of the coating obtained from the coating composition. In various examples, the coating composition may include from about 1% to about 80%, or from about 2% to about 75%, or from about 3% to about 70%, or 4% to about 65% by weight, or from about 5% to about 50% by weight, or from about 5% to about 45% by weight, or from about 10% to about 50% by weight, or from about 10% to about 45% by weight, or from about 10% to about 40% by weight, or from about 10% to about 35% by weight, or from about 15% to about 40% by weight, or from about 15% to about 35% by weight of the aryl-modified hyperbranched polyol based on the total amount of film-forming materials (also called the binder or vehicle of the coating composition).

The coating composition may include other reactive resins or polymers. Examples of useful resins or polymers include (meth)acrylate polymers (also known as acrylic polymers or resins), polyesters, polyethers, polyurethanes, polyols based on natural oils, such as those available under the trademark Polycins from Vertellus Specialties Inc, Indianapolis, Ind., for example a polyol based on castor oil, polysiloxanes, and those described in Mormile et al., U.S. Pat. No. 5,578,675; Lane et al U.S. Patent Application Publ'n No. 2011/0135,832; and Groenewolt et al., U.S. Patent Application Publ'n No. 2013/0136865, each of which is incorporated herein by reference. The other resins or polymers may have functionality reactive with the crosslinker for the aryl-modified hyperbranched polyol, or that the coating composition may contain a further crosslinker for the other resins or polymer. In certain preferred examples, the coating composition includes a further resin or polymer having hydroxyl groups, carbamate groups, or a combination of such groups. In various embodiments, the coating composition contains a hydroxyl-functional acrylic polymer, hydroxyl-functional polyester, or hydroxyl-functional polyurethane.

Polyvinyl polyols, such as acrylic (polyacrylate) polyol polymers that may be used as the hydroxy-functional material. Acrylic polymers or polyacrylate polymers may be copolymers of both acrylic and methacrylic monomers as well as other copolymerizable vinyl monomers. The term “(meth)acrylate” is used for convenience to designate either or both acrylate, and methacrylate, and the term “(meth)acrylic” is used for convenience to designate either or both acrylic and methacrylic.

Hydroxyl-containing monomers include hydroxy alkyl esters of acrylic or methacrylic acid. Nonlimiting examples of hydroxyl-functional monomers include hydroxyethyl(meth)acrylate, hydroxypropyl(meth)acrylates, hydroxybutyl(meth)acrylates, hydroxyhexyl(meth)acrylates, propylene glycol mono(meth)acrylate, 2,3-dihydroxypropyl(meth)acrylate, pentaerythritol mono(meth)acrylate, polypropylene glycol mono(meth)acrylates, polyethylene glycol mono(meth)acrylates, reaction products of these with epsilon-caprolactone, and other hydroxyalkyl(meth)acrylates having branched or linear alkyl groups of up to about 10 carbons, and mixtures of these, where the term “(meth)acrylate” indicates either or both of the methacrylate and acrylate esters. Generally, at least about 5% by weight hydroxyl-functional monomer is included in the polymer. Hydroxyl groups on a vinyl polymer such as an acrylic polymer can be generated by other means, such as, for example, the ring opening of a glycidyl group, for example from copolymerized glycidyl methacrylate, by an organic acid or an amine.

Hydroxyl functionality may also be introduced through thio-alcohol compounds, including, without limitation, 3-mercapto-1-propanol, 3-mercapto-2-butanol, 11-mercapto-1-undecanol, 1-mercapto-2-propanol, 2-mercaptoethanol, 6-mercapto-l-hexanol, 2-mercaptobenzyl alcohol, 3-mercapto-1,2-proanediol, 4-mercapto-1-butanol, and combinations of these. Any of these methods may be used to prepare a useful hydroxyl-functional acrylic polymer.

Examples of suitable comonomers that may be used include, without limitation, α,β-ethylenically unsaturated monocarboxylic acids containing 3 to 5 carbon atoms such as acrylic, methacrylic, and crotonic acids and the alkyl and cycloalkyl esters, nitriles, and amides of acrylic acid, methacrylic acid, and crotonic acid; α,β-ethylenically unsaturated dicarboxylic acids containing 4 to 6 carbon atoms and the anhydrides, monoesters, and diesters of those acids; vinyl esters, vinyl ethers, vinyl ketones, and aromatic or heterocyclic aliphatic vinyl compounds. Representative examples of suitable esters of acrylic, methacrylic, and crotonic acids include, without limitation, those esters from reaction with saturated aliphatic alcohols containing 1 to 20 carbon atoms, such as methyl, ethyl, propyl, isopropyl, n-butyl, isobutyl, tert-butyl, hexyl, 2-ethylhexyl, dodecyl, 3,3,5-trimethylhexyl, stearyl, lauryl, cyclohexyl, alkyl-substituted cyclohexyl, alkanol-substituted cyclohexyl, such as 2-tert-butyl and 4-tert-butyl cyclohexyl, 4-cyclohexyl-l-butyl, 2-tert-butyl cyclohexyl, 4-tert-butyl cyclohexyl, 3,3,5,5,-tetramethyl cyclohexyl, tetrahydrofurfuryl, and isobornyl acrylates, methacrylates, and crotonates; unsaturated dialkanoic acids and anhydrides such as fumaric, maleic, itaconic acids and anhydrides and their mono- and diesters with alcohols such as methanol, ethanol, propanol, isopropanol, butanol, isobutanol, and tert-butanol, like maleic anhydride, maleic acid dimethyl ester and maleic acid monohexyl ester; vinyl acetate, vinyl propionate, vinyl ethyl ether, and vinyl ethyl ketone; styrene, a-methyl styrene, vinyl toluene, 2-vinyl pyrrolidone, and p-tert-butylstyrene.

The acrylic polymer may be prepared using conventional techniques, such as by heating the monomers in the presence of a polymerization initiating agent and optionally a chain transfer agent. The polymerization may be carried out in solution, for example. Typical initiators are organic peroxides such as dialkyl peroxides such as di-t-butyl peroxide, peroxyesters such as t-butyl peroxy 2-ethylhexanoate, and t-butyl peracetate, peroxydicarbonates, diacyl peroxides, hydroperoxides such as t-butyl hydroperoxide, and peroxyketals; azo compounds such as 2,2′azobis(2-methylbutanenitrile) and 1,1′-azobis(cyclohexanecarbonitrile); and combinations of these. Typical chain transfer agents are mercaptans such as octyl mercaptan, n- or tert-dodecyl mercaptan; halogenated compounds, thiosalicylic acid, mercaptoacetic acid, mercaptoethanol and the other thiol alcohols already mentioned, and dimeric alpha-methyl styrene.

The reaction is usually carried out at temperatures from about 20° C. to about 200° C. The reaction may conveniently be done at the temperature at which the solvent or solvent mixture refluxes, although with proper control a temperature below the reflux may be maintained. The initiator should be chosen to match the temperature at which the reaction is carried out, so that the half-life of the initiator at that temperature should preferably be no more than about thirty minutes. Further details of addition polymerization generally and of polymerization of mixtures including (meth)acrylate monomers is readily available in the polymer art. The solvent or solvent mixture is generally heated to the reaction temperature and the monomers and initiator(s) are added at a controlled rate over a period of time, usually between 2 and 6 hours. A chain transfer agent or additional solvent may be fed in also at a controlled rate during this time. The temperature of the mixture is then maintained for a period of time to complete the reaction. Optionally, additional initiator may be added to ensure complete conversion.

Oligomeric and polymeric ethers may be used, including diethylene glycol, triethylene glycol, tetraethylene glycol, pentaethylene glycol, dipropylene glycol, tripropylene glycol, linear and branched polyethylene glycols, polypropylene glycols, and block copolymers of poly(ethylene oxide-co-propylene oxide). Other polymeric polyols may be obtained by reacting a polyol initiator, e.g., a diol such as 1,3-propanediol or ethylene or propylene glycol or a polyol such as trimethylolpropane or pentaerythritol, with a lactone or alkylene oxide chain-extension reagent. Lactones that can be ring opened by active hydrogen are well known in the art. Examples of suitable lactones include, without limitation, ε-caprolactone, γ-caprolactone, β-butyrolactone, β-propriolactone, γ-butyrolactone, α-methyl-γ-butyrolactone, β-methyl-γ-butyrolactone, γ-valerolactone, δ-valerolactone, γ-decanolactone, δ-decanolactone, γ-nonanoic lactone, γ-octanoic lactone, and combinations of these. In one preferred embodiment, the lactone is ε-caprolactone. Useful catalysts include those mentioned above for polyester synthesis. Alternatively, the reaction can be initiated by forming a sodium salt of the hydroxyl group on the molecules that will react with the lactone ring. Similar polyester polyols may be obtained by reacting polyol initiator molecules with hydroxy acids, such as 12-hydroxystearic acid.

In other embodiments, a polyol initiator compound may be reacted with an oxirane-containing compound to produce a polyether diol to be used in the polyurethane elastomer polymerization. Alkylene oxide polymer segments include, without limitation, the polymerization products of ethylene oxide, propylene oxide, 1,2-cyclohexene oxide, 1-butene oxide, 2-butene oxide, 1-hexene oxide, tert-butylethylene oxide, phenyl glycidyl ether, 1-decene oxide, isobutylene oxide, cyclopentene oxide, 1-pentene oxide, and combinations of these. The oxirane-containing compound is preferably selected from ethylene oxide, propylene oxide, butylene oxide, tetrahydrofuran, and combinations of these. The alkylene oxide polymerization is typically base-catalyzed. The polymerization may be carried out, for example, by charging the hydroxyl-functional initiator compound and a catalytic amount of caustic, such as potassium hydroxide, sodium methoxide, or potassium tert-butoxide, and adding the alkylene oxide at a sufficient rate to keep the monomer available for reaction. Two or more different alkylene oxide monomers may be randomly copolymerized by coincidental addition or polymerized in blocks by sequential addition. Homopolymers or copolymers of ethylene oxide or propylene oxide are preferred. Tetrahydrofuran may be polymerized by a cationic ring-opening reaction using such counterions as SbF6^{—}, AsF₆^{—}, PF₆^{—}, SbCl₆^{—}, BF₄^{—}, CF₃SO₃^{—}, FSO₃, and ClO₄^{—}. Initiation is by formation of a tertiary oxonium ion. The polytetrahydrofuran segment can be prepared as a “living polymer” and terminated by reaction with the hydroxyl group of a diol such as any of those mentioned above. Polytetrahydrofuran is also known as polytetramethylene ether glycol (PTMEG). Any of the polyols mentioned above maybe employed as the polyol initiator and extended in this fashion.

Nonlimiting examples of suitable polycarbonate polyols that might be used include those prepared by the reaction of polyols with dialkyl carbonates (such as diethyl carbonate), diphenyl carbonate, or dioxolanones (such as cyclic carbonates having five- and six-member rings) in the presence of catalysts like alkali metal, tin catalysts, or titanium compounds. Useful polyols include, without limitation, any of those already mentioned. Aromatic polycarbonates are usually prepared from reaction of bisphenols, e.g., bisphenol A, with phosgene or diphenyl carbonate. Aliphatic polycarbonates may be preferred for a higher resistance to yellowing, particularly when the carbamate-functional material is used in an automotive OEM or refinish topcoat.

Polyesters polyols may be prepared by reacting: (a) polycarboxylic acids or their esterifiable derivatives, together if desired with monocarboxylic acids, (b) polyols, together if desired with monofunctional alcohols, and (c) if desired, other modifying components. Nonlimiting examples of polycarboxylic acids and their esterifiable derivatives include phthalic acid, isophthalic acid, terephthalic acid, halophthalic acids such as tetrachloro- or tetrabromophthalic acid, adipic acid, glutaric acid, azelaic acid, sebacic acid, fumaric acid, maleic acid, trimellitic acid, pyromellitic acid, tetrahydrophthalic acid, hexahydrophthalic acid, 1,2-cyclohexanedicarboxlic acid, 1,3-cyclohexane-discarboxlic acid, 1,4-cyclohexane-dicarboxlic acid, 4-methylhexahydrophthalic acid, endomethylenetetrahydropthalic acid, tricyclodecanedicarboxlic acid, endoethylenehexahydropthalic acid, camphoric acid, cyclohexanetetracarboxlic acid, and cyclobutanetetracarboxylic acid. The cycloaliphatic polycarboxylic acids may be employed either in their cis or in their trans form or as a mixture of the two forms. Esterifiable derivatives of these polycarboxylic acids include their single or multiple esters with aliphatic alcohols having 1 to 4 carbon atoms or hydroxy alcohols having up to 4 carbon atoms, preferably the methyl and ethyl ester, as well as the anhydrides of these polycarboxylic acids, where they exist. Nonlimiting examples of suitable monocarboxylic acids that can be used together with the polycarboxylic acids include benzoic acid, tert-butylbenzoic acid, lauric acid, isonoanoic acid and fatty acids of naturally occurring oils. Nonlimiting examples of suitable polyols include any of those already mentioned above, such as ethylene glycol, butylene glycol, neopentyl glycol, propanediols, butanediols, hexanediols, diethylene glycol, cyclohexanediol, cyclohexanedimethanol, trimethylpentanediol, ethylbutylpropanediol ditrimethylolpropane, trimethylolethane, trimethylolpropane, glycerol, pentaerythritol, dipentaerythritol, tris-hydroxyethyl isocyanate, polyethylene glycol, polypropylene glycol, and polyols derived from natural oils. Nonlimiting examples of monoalcohols that may be used together with the polyols include butanol, octanol, lauryl alcohol, and ethoxylated and propoxylated phenols. Nonlimiting examples of suitable modifying components include compounds which contain a group which is reactive with respect to the functional groups of the polyester, including polyisocyanates and/or diepoxide compounds, and also if desired, monoisocyanates and/or monoepoxide compounds. The polyester polymerization may be carried out by known standard methods. This reaction is conventionally carried out at temperatures of between 180° C. and 280° C., in the presence if desired of an appropriate esterification catalyst. Typical catalysts for the esterification polymerization are protonic acids, Lewis acids, titanium alkoxides, and dialkyltin oxides, for example lithium octanoate, dibutyltin oxide, dibutyltin dilaurate, para-toluenesulfonic acid under reflux with small quantities of a suitable solvent as entraining agent such as an aromatic hydrocarbon, for example xylene, or a (cyclo)aliphatic hydrocarbon, for example cyclohexane.

Polyurethanes having hydroxyl functional groups may also be used in the coating compositions along with the aryl-modified hyperbranched polyol. Examples of suitable polyurethane polyols include polyester-polyurethanes, polyether-polyurethanes, and polycarbonate-polyurethanes, including, without limitation, polyurethanes polymerized using as polymeric diol reactants polyethers and polyesters including polycaprolactone polyesters or polycarbonate diols. These polymeric diol-based polyurethanes are prepared by reaction of the polymeric diol (polyester diol, polyether diol, polycaprolactone diol, polytetrahydrofuran diol, or polycarbonate diol), one or more polyisocyanates, and, optionally, one or more chain extension compounds. Chain extension compounds, as the term is being used, are compounds having two or more functional groups, preferably two functional groups, reactive with isocyanate groups, such as the diols, amino alcohols, and diamines. Preferably the polymeric diol-based polyurethane is substantially linear (i.e., substantially all of the reactants are difunctional).

Diisocyanates used in making the polyurethane polyols may be aromatic, aliphatic, or cycloaliphatic. Useful diisocyanate compounds include, without limitation, isophorone diisocyanate (IPDI), methylene bis-4-cyclohexyl isocyanate (H₁₂MDI), cyclohexyl diisocyanate (CHDI), m-tetramethyl xylene diisocyanate (m-TMXDI), p-tetramethyl xylene diisocyanate (p-TMXDI), 4,4′-methylene diphenyl diisocyanate (MDI, also known as 4,4′-diphenylmethane diisocyanate), 2,4- or 2,6-toluene diisocyanate (TDI), ethylene diisocyanate, 1,2-diisocyanatopropane, 1,3-diisocyanatopropane, 1,6-diisocyanatohexane (hexamethylene diisocyanate or HDI), 1,4-butylene diisocyanate, lysine diisocyanate, meta-xylylenediioscyanate and para-xylylenediisocyanate, 4-chloro-1,3-phenylene diisocyanate, 1,5-tetrahydro-naphthalene diisocyanate, 4,4′-dibenzyl diisocyanate, and xylylene diisocyanate (XDI), and combinations of these. Nonlimiting examples of higher-functionality polyisocyanates that may be used in limited amounts to produce branched thermoplastic polyurethanes (optionally along with monofunctional alcohols or monofunctional isocyanates) include 1,2,4-benzene triisocyanate, 1,3,6-hexamethylene triisocyanate, 1,6,11-undecane triisocyanate, bicycloheptane triisocyanate, triphenylmethane-4,4′,4″-triisocyanate, isocyanurates of diisocyanates, biurets of diisocyanates, allophanates of diisocyanates, and the like. These and other diisocyanates and other higher-functional isocyanates can also be used to make urethane cured coatings by reaction with hydroxyl groups of the aryl-modified hyperbranched polyol. At a minimum, the aryl-modified hyperbranched polyol has hydroxyl groups, depending on the embodiment, as a result of the reaction in steps (c1), (c2), (e1), and (e2) where epoxide groups are reacted with carboxylic acid functional groups or as a result of the reaction steps (b1), (d1), (c2a), (d2a), (e1a), and (e2a), where the poly-lactone chains would be formed as a result of ring-opening reactions there.

In various embodiments, polymeric diols for co-reacting with diisocyanates preferably have a weight average molecular weight of at least about 500, more preferably at least about 1000, and even more preferably at least about 1800 and a weight average molecular weight of up to about 10,000, but polymeric diols having weight average molecular weights of up to about 5000, especially up to about 4000, may also be preferred. The polymeric diol advantageously has a weight average molecular weight in the range from about 500 to about 10,000, preferably from about 1000 to about 5000, and more preferably from about 1500 to about 4000. The weight average molecular weights may be determined by ASTM D-4274.

The reaction of the polyisocyanate, polymeric diol, and diol or other chain extension agent is typically carried out at an elevated temperature in the presence of a suitable catalyst, for example tertiary amines, zinc salts, and manganese salts. The ratio of polymeric diol, such as polyester diol, to extender can be varied within a relatively wide range depending largely on the desired hardness or flexibility of the final polyurethane elastomer. For example, the equivalent proportion of polyester diol to extender may be within the range of 1:0 to 1:12 and, more preferably, from 1:1 to 1:8. Preferably, the diisocyanate(s) employed are proportioned such that the overall ratio of equivalents of isocyanate to equivalents of active hydrogen containing materials is within the range of 1:1 to 1:1.05, and more preferably, 1:1 to 1:1.02. The polymeric diol segments typically are from about 35% to about 65% by weight of the polyurethane polymer, and preferably from about 35% to about 50% by weight of the polyurethane polymer.

A polysiloxane polyol may be made by hydrosilylating a polysiloxane containing silicon hydrides with an alkyenyl polyoxyalkylene alcohol containing two or three terminal primary hydroxyl groups, for example allylic polyoxyalkylene alcohols such as trimethylolpropane monoallyl ether and pentaerythritol monoallyl ether.

Any of the polyol resins and polymers described above may be derivatized to have carbamate groups according to known methods, for example by reaction of a hydroxyl-functional material with an alkyl carbamate, for example methyl carbamate or butyl carbamate, through what is referred to as “transcarbamation” or “transcarbamoylation.” In other methods of forming carbamate-functional resins and polymers for use in the coating compositions, the resin and polymers may be polymerized using a carbamate-functional monomer.

The coating composition containing the aryl-modified hyperbranched polyol and optional further active hydrogen-functional resin or polymer may also include at least one crosslinker or curing agent reactive with hydroxyl groups, such as aminoplast crosslinkers having active methylol, methylalkoxy or butylalkoxy groups; polyisocyanate crosslinkers, which may have blocked or unblocked isocyanate groups; polyanhydrides; and polyepoxide functional crosslinkers or curing agents, which could be reactive with the hydroxyls as well as with any carboxylic acid groups of the aryl-modified hyperbranched polyols.

Aminoplasts, or amino resins, are described in Encyclopedia of Polymer Science and Technology vol. 1, p. 752-789 (1985), the disclosure of which is hereby incorporated by reference. An aminoplast is obtained by reaction of an activated nitrogen with a lower molecular weight aldehyde, optionally with further reaction with an alcohol (preferably a mono-alcohol with one to four carbon atoms such as methanol, isopropanol, n-butanol, isobutanol, etc.) to form an ether group. Preferred examples of activated nitrogens are activated amines such as melamine, benzoguanamine, cyclohexylcarboguanamine, and acetoguanamine; ureas, including urea itself, thiourea, ethyleneurea, dihydroxyethyleneurea, and guanylurea; glycoluril; amides, such as dicyandiamide; and carbamate-functional compounds having at least one primary carbamate group or at least two secondary carbamate groups. The activated nitrogen is reacted with a lower molecular weight aldehyde. The aldehyde may be selected from formaldehyde, acetaldehyde, crotonaldehyde, benzaldehyde, or other aldehydes used in making aminoplast resins, although formaldehyde and acetaldehyde, especially formaldehyde, are preferred. The activated nitrogen groups are at least partially alkylolated with the aldehyde, and may be fully alkylolated; preferably the activated nitrogen groups are fully alkylolated. The reaction may be catalyzed by an acid, e.g. as taught in U.S. Pat. No. 3,082,180, which is incorporated herein by reference.

The optional alkylol groups formed by the reaction of the activated nitrogen with aldehyde may be partially or fully etherified with one or more monofunctional alcohols. Suitable examples of the monofunctional alcohols include, without limitation, methanol, ethanol, n-propanol, isopropanol, n-butanol, isobutanol, tert-butyl alcohol, benzyl alcohol, and so on. Monofunctional alcohols having one to four carbon atoms and mixtures of these are preferred. The etherification may be carried out, for example, the processes disclosed in U.S. Pat. Nos. 4,105,708 and 4,293,692 incorporate the disclosures of which incorporated herein by reference. The aminoplast may be at least partially etherified, and in various embodiments the aminoplast is fully etherified. For example, the aminoplast compounds may have a plurality of methylol and/or etherified methylol, butylol, or alkylol groups, which may be present in any combination and along with unsubstituted nitrogen hydrogens. Examples of suitable curing agent compounds include, without limitation, melamine formaldehyde resins, including monomeric or polymeric melamine resins and partially or fully alkylated melamine resins, and urea resins (e.g., methylol ureas such as urea formaldehyde resin, and alkoxy ureas such as butylated urea formaldehyde resin). One nonlimiting example of a fully etherified melamine-formaldehyde resin is hexamethoxymethyl melamine.

The alkylol groups are capable of self-reaction to form oligomeric and polymeric aminoplast crosslinking agents. Useful materials are characterized by a degree of polymerization. For melamine formaldehyde resins, it is preferred to use resins having a number average molecular weight less than about 2000, more preferably less than 1500, and even more preferably less than 1000.

A coating composition including aminoplast crosslinking agents may further include a strong acid catalyst to enhance the cure reaction. Such catalysts are well known in the art and include, for example, para-toluenesulfonic acid, dinonylnaphthalene disulfonic acid, dodecylbenzenesulfonic acid, phenyl acid phosphate, monobutyl maleate, butyl phosphate, and hydroxy phosphate ester. Strong acid catalysts are often blocked, e.g. with an amine.

Particularly for refinish coatings, polyisocyanate crosslinkers are commonly used. Examples of suitable polyisocyanate crosslinkers include, without limitation, alkylene polyisocyanates such as hexamethylene diisocyanate, 4- and/or 2,4,4-trimethylhexamethylene diisocyanate, dodecamethylene diisocyanate, 1,4-diisocyanatocyclohexane, 1-isocyanato-3,3,5-trimethyl-5-isocyanatomethylcyclohexane (isophorone diisocyanate), 2,4′- and/or 4,4′-diisocyanatodicyclohexylmethane, 3-isocyanato-methyl-3,5,5-trimethyl cyclohexyl isocyanate, aromatic polyisocyanates such as 2,4′- and/or 4,4′-diisocyanatodiphenylmethane, 2,4- and/or 2,6-diisocyanatotoluene, naphthylene diisocyanate, and mixtures of these polyisocyanates. Generally, polyisocyanates having three or more isocyanate groups are used; these may be derivatives or adducts of diisocyanates. Useful polyisocyanates may be obtained by reaction of an excess amount of an isocyanate with water, a polyol (for example, ethylene glycol, propylene glycol, 1,3-butylene glycol, neopentyl glycol, 2,2,4-trimethyl-1,3-pentane diol, hexamethylene glycol, cyclohexane dimethanol, hydrogenated bisphenol A, trimethylolpropane, trimethylolethane, 1,2,6-hexanetriol, glycerine, sorbitol or pentaerythritol), or by the reaction of the isocyanate with itself to give an isocyanurate. Examples include biuret-group-containing polyisocyanates, such as those described, for example, in U.S. Pat. No. 3,124,605 and U.S. Pat. No. 3,201,372 or DE-OS 1,101,394; isocyanurate-group-containing polyisocyanates, such as those described, for example, in U.S. Pat. No. 3,001,973, DE-PS 1,022,789, 1,222,067 and 1,027,394 and in DE-OS 1,929,034 and 2,004,048; urethane-group-containing polyisocyanates, such as those described, for example, in DE-OS 953,012, BE-PS 752,261 or U.S. Pat. Nos. 3,394,164 and 3,644,457; carbodiimide group-containing polyisocyanates, such as those described in DE-PS 1,092,007, U.S. Pat. No. 3,152,162. and DE-OS 2,504,400, 2,537,685 and 2,552,350; allophanate group-containing polyisocyanates, such as those described, for example, in GB-PS 994,890, BE-PS 761,626 and NL-05 7,102,524; and uretdione group-containing polyisocyanates, such as those described in EP-A 0,377,177, each reference being incorporated herein by reference.

Such isocyanate crosslinkers for refinish coating compositions are commonly stored separately and combined with the hydroxyl-functional film-forming components shortly before application. For example, a two-part or two-pack or two-component refinish coating composition may include in a crosslinking part, package, or component one of aliphatic biurets and isocyanurates, such as the isocyanurates of hexamethylene diisocyanate and isophorone diisocyanate.

Curing catalysts for the urethane reaction such as tin catalysts can be used in the coating composition. Typical examples are without limitation, tin and bismuth compounds including dibutyltin dilaurate, dibutyltin oxide, and bismuth octoate. When used, catalysts are typically present in amounts of about 0.05 to 2 percent by weight tin based on weight of total nonvolatile vehicle.

A dianhydride may also be used to crosslink the aryl-modified hyperbranched polyol. Nonlimiting examples of di-cyclic carboxylic anhydrides include pyromellitic dianhydride, ethylenediaminetetraacetic dianhydride, cyclobutane-1,2,3,4-tetracarboxylic dianhydride, 3,3′,4,4′-biphenyltetracarboxylic dianhydride, tetrahydrofurane-2,3,4,5-tetracarboxylic dianhydride, and cyclohexane-1,2,4,5-tetracarboxylic acid dianhydride.

Polyepoxide crosslinking agents include acrylic polymers having epoxide groups, for example copolymers of allyl glycidyl ether, glycidyl acrylate, or glycidyl methacrylate, as well as polyglycidyl esters and ethers of polyol and polycarboxylic acids.

The coating composition made with the aryl-modified hyperbranched polyol may further include solvents, pigments, fillers, or customary additives.

A solvent may optionally be utilized in the coating compositions. Although the coating composition may be formulated, for example, in the form of a powder, it is often desirable that the composition be in a substantially liquid state, which can be accomplished with the use of a solvent to either dissolve or disperse the aryl-modified hyperbranched polyol, crosslinker, and other film-forming material or materials. In general, depending on the solubility characteristics of the components, the solvent can be any organic solvent and/or water. In one preferred embodiment, the solvent is a polar organic solvent. For example, the solvent may be a polar aliphatic solvent or polar aromatic solvent. Among useful solvents are ketone, ester, acetate, aprotic amide, aprotic sulfoxide, and aprotic amine solvents. Examples of specific useful solvents include ketones, such as acetone, methyl ethyl ketone, methyl amyl ketone, methyl isobutyl ketone, esters such as ethyl acetate, butyl acetate, pentyl acetate, ethyl ethoxypropionate, ethylene glycol butyl ether acetate, propylene glycol monomethyl ether acetate, aliphatic and/or aromatic hydrocarbons such as toluene, xylene, solvent naphtha, and mineral spirits, ethers such as glycol ethers like propylene glycol monomethyl ether, alcohols such as ethanol, propanol, isopropanol, n-butanol, isobutanol, and tert-butanol, nitrogen-containing compounds such as N-methyl pyrrolidone and N-ethyl pyrrolidone, and combinations of these. In example embodiments, the liquid medium is water or a mixture of water with small amounts of organic water-soluble or water-miscible co-solvents. The solvent in the coating composition may be present in an amount of from about 0.01 weight percent to about 99 weight percent, or in an amount of from about 10 weight percent to about 60 weight percent, or in an amount of from about 30 weight percent to about 50 weight percent.

The coating compositions contain pigments, including special effect pigments, and optionally fillers. The coating compositions are formulated as basecoat topcoats, monocoat topcoats, or primers. Nonlimiting examples of special effect pigments that may be utilized in basecoat and monocoat topcoat coating compositions include metallic, pearlescent, and color-variable effect flake pigments. Metallic (including pearlescent, and color-variable) topcoat colors are produced using one or more special flake pigments. Metallic colors are generally defined as colors having gonioapparent effects. For example, the American Society of Testing Methods (ASTM) document F284 defines metallic as “pertaining to the appearance of a gonioapparent material containing metal flake.” Metallic basecoat colors may be produced using metallic flake pigments like aluminum flake pigments, coated aluminum flake pigments, copper flake pigments, zinc flake pigments, stainless steel flake pigments, and bronze flake pigments and/or using pearlescent flake pigments including treated micas like titanium dioxide-coated mica pigments and iron oxide-coated mica pigments to give the coatings a different appearance (degree of reflectance or color) when viewed at different angles. Metal flakes may be cornflake type, lenticular, or circulation-resistant; micas may be natural, synthetic, or aluminum oxide type. Flake pigments do not agglomerate and are not ground under high shear because high shear would break or bend the flakes or their crystalline morphology, diminishing or destroying the gonioapparent effects. The flake pigments are satisfactorily dispersed in a binder component by stirring under low shear. The flake pigment or pigments may be included in the high solids coating composition in an amount of about 0.01 wt. % to about 50 wt. % or about 15 wt. % to about 25 wt. %, in each case based on total binder weight. Nonlimiting examples of commercial flake pigments include PALIOCROME® pigments, available from BASF Corporation.

Nonlimiting examples of other suitable pigments and fillers that may be utilized in basecoat and monocoat topcoat coating compositions include inorganic pigments such as titanium dioxide, barium sulfate, carbon black, ocher, sienna, umber, hematite, limonite, red iron oxide, transparent red iron oxide, black iron oxide, brown iron oxide, chromium oxide green, strontium chromate, zinc phosphate, silicas such as fumed silica, calcium carbonate, talc, barytes, ferric ammonium ferrocyanide (Prussian blue), and ultramarine, and organic pigments such as metallized and non-metallized azo reds, quinacridone reds and violets, perylene reds, copper phthalocyanine blues and greens, carbazole violet, monoarylide and diarylide yellows, benzimidazolone yellows, tolyl orange, naphthol orange, nanoparticles based on silicon dioxide, aluminum oxide or zirconium oxide, and so on. The pigment or pigments are preferably dispersed in a resin or polymer or with a pigment dispersant, such as binder resins of the kind already described, according to known methods. In general, the pigment and dispersing resin, polymer, or dispersant are brought into contact under a shear high enough to break the pigment agglomerates down to the primary pigment particles and to wet the surface of the pigment particles with the dispersing resin, polymer, or dispersant. The breaking of the agglomerates and wetting of the primary pigment particles are important for pigment stability and color development. Pigments and fillers may be utilized in amounts typically of up to about 60% by weight, based on total weight of the coating composition. The amount of pigment used depends on the nature of the pigment and on the depth of the color and/or the intensity of the effect it is intended to produce, and also by the dispersibility of the pigments in the pigmented coating composition. The pigment content, based in each case on the total weight of the pigmented coating composition, is preferably 0.5% to 50%, more preferably 1% to 30%, very preferably 2% to 20%, and more particularly 2.5% to 10% by weight.

The aryl-modified hyperbranched polyols may also be used in transparent pigmented topcoat coating compositions, (tinted clearcoats), as well as in clearcoat coating compositions that do not include a pigment. Certain embodiments of the invention are particularly suited for stabilizing the ultra-small organic pigment particles required for making transparent pigmented topcoat coatings. Such tinted clearcoats are being used more frequently to expand the palette of decorative effects which can be achieved when coating vehicles or other surfaces which it might be desirable to decorate with a coating. Certain other embodiments of aryl-modified hyperbranched polyols may be preferred to help impart non-colorant related effects to non-pigmented clearcoats.

Additional desired, customary coating additives agents may be included, for example, surfactants, stabilizers, wetting agents, dispersing agents, adhesion promoters, UV absorbers, hindered amine light stabilizers such as HALS compounds, benzotriazoles or oxalanilides; free-radical scavengers; slip additives; defoamers; reactive diluents, of the kind which are common knowledge from the prior art; wetting agents such as siloxanes, fluorine compounds, carboxylic monoesters, phosphoric esters, polyacrylic acids and their copolymers, for example polybutyl acrylate, or polyurethanes; adhesion promoters such as tricyclodecanedimethanol; flow control agents; film-forming assistants such as cellulose derivatives; rheology control additives, such as the additives known from patents WO 94/22968, EP-A-0 276 501, EP-A-0 249 201 or WO 97/12945; crosslinked polymeric microparticles, as disclosed for example in EP-A-0 008 127; inorganic phyllosilicates such as aluminum-magnesium silicates, sodium-magnesium and sodium-magnesium-fluorine-lithium phyllosilicates of the montmorillonite type; silicas such as Aerosils®; or synthetic polymers containing ionic and/or associative groups such as polyvinyl alcohol, poly(meth)acrylamide, poly(meth)acrylic acid, polyvinylpyrrolidone, styrene-maleic anhydride copolymers or ethylene-maleic anhydride copolymers and their derivatives, or hydrophobically modified ethoxylated urethanes or polyacrylates; flame retardant; and so on. Typical coating compositions include one or a combination of such additives.

Coating compositions can be coated by any of a number of techniques well known in the art. These include, for example, spray coating, dip coating, roll coating, curtain coating, knife coating, spreading, pouring, dipping, impregnating, trickling or rolling, and the like. For automotive body panels, spray coating is typically used. Preference is given to employing spray application methods, such as compressed-air spraying, airless spraying, high-speed rotation, electrostatic spray application, alone or in conjunction with hot spray application such as hot-air spraying, for example.

The coating compositions and coating systems described herein are employed in particular in the technologically and esthetically particularly demanding field of automotive OEM finishing and also of automotive refinish. The coating compositions can be used in both single-stage and multistage coating methods, particularly in methods where a pigmented basecoat or monocoat coating layer is first applied to an uncoated or precoated substrate and afterward another coating layer may optionally be applied when the pigmented film is a basecoat coating. The invention, accordingly, also provides multicoat coating systems comprising at least one pigmented basecoat and may have least one clearcoat disposed thereon, wherein either the clearcoat or the basecoat has been or both have been produced from the coating composition containing the aryl-modified hyperbranched polyol as disclosed herein. Both the basecoat and the clearcoat coating composition can include the disclosed aryl-modified hyperbranched polyol.

The applied coating compositions can be cured after a certain rest time or “flash” period. The rest time serves, for example, for the leveling and devolatilization of the coating films or for the evaporation of volatile constituents such as solvents. The rest time may be assisted or shortened by the application of elevated temperatures or by a reduced humidity, provided this does not entail any damage or alteration to the coating films, such as premature complete crosslinking, for instance. The thermal curing of the coating compositions has no peculiarities in terms of method but instead takes place in accordance with the typical, known methods such as heating in a forced-air oven or irradiation with IR lamps. The thermal cure may also take place in stages. Another preferred curing method is that of curing with near infrared (NIR) radiation. Although various methods of curing may be used, heat curing is preferred. Generally, heat curing is effected by exposing the coated article to elevated temperatures provided primarily by radiative heat sources. After application, the applied coating layer is cured, for example with heat at temperatures from 30 to 200° C., or from 40 to 190° C., or from 50 to 180° C., for a time of 1 min up to 10 h, more preferably 2 min up to 5 h, and in particular 3 min to 3 h, although longer cure times may also be employed at the temperatures employed for automotive refinish, which are preferably between 30 and 90° C. The aryl-modified hyperbranched polyol can be used for both refinish coatings and for original finish coatings that are cured at higher temperatures. A typical method for applying a refinish coating composition includes application and drying with cure at room temperature or at an elevated temperature between 30 and 90° C. OEM coatings are typically cured at higher temperatures, for example from about 110 to about 135° C. The curing time will vary depending on the particular components used, and physical parameters such as the thickness of the layers, however, typical curing times range from about 15 to about 60 minutes, and preferably about 15-25 minutes for blocked acid catalyzed systems and about 10-20 minutes for unblocked acid catalyzed systems.

Cured basecoat layers formed may have a thickness of from about 5 to about 75 μm, depending mainly upon the color desired and the thickness needed to form a continuous layer that will provide the color. Cured clearcoat layers formed typically have thicknesses of from about 30 μm to about 65 μm.

The coating composition can be applied onto many different types of substrates, including metal substrates such as bare steel, phosphated steel, galvanized steel, or aluminum; and non-metallic substrates, such as plastics and composites. The substrate may also be any of these materials having upon it already a layer of another coating, such as a layer of an electrodeposited primer, primer surfacer, and/or basecoat, cured or uncured.

The substrate may be first primed with an electrodeposition (electrocoat) primer. The electrodeposition composition can be any electrodeposition composition used in automotive vehicle coating operations. Non-limiting examples of electrocoat compositions include electrocoating compositions sold by BASF. Electrodeposition coating baths usually comprise an aqueous dispersion or emulsion including a principal film-forming epoxy resin having ionic stabilization (e.g., salted amine groups) in water or a mixture of water and organic cosolvent. Emulsified with the principal film-forming resin is a crosslinking agent that can react with functional groups on the principal resin under appropriate conditions, such as with the application of heat, and so cure the coating. Suitable examples of crosslinking agents, include, without limitation, blocked polyisocyanates. The electrodeposition coating compositions usually include one or more pigments, catalysts, plasticizers, coalescing aids, antifoaming aids, flow control agents, wetting agents, surfactants, UV absorbers, HALS compounds, antioxidants, and other additives.

The electrodeposition coating composition is preferably applied to a dry film thickness of 10 to 35 μm. After application, the coated vehicle body is removed from the bath and rinsed with deionized water. The coating may be cured under appropriate conditions, for example by baking at from about 135° C. to about 190° C. for between about 15 and about 60 minutes.

Because the coatings of the invention produced from the coating compositions of the invention adhere excellently even to electrocoats, surfacer coats, basecoat systems or typical, known clearcoat systems that have already cured, they are outstandingly suitable not only for use in automotive OEM finishing but also for automotive refinish or for the modular scratchproofing of automobile bodies that have already been painted.

A coating produced from the coating composition containing the aryl-modified hyperbranched has excellent durability due to the incorporation of the aryl groups, low VOC content, and excellent flexibility, particularly at low temperatures.

### EXAMPLES

### Example 1. Synthesis of a Water-Reducible Aryl-Modified Hyperbranched Polyol

A reactor was charged with 17.578 parts by weight of 2,2′-(oxybis(methylene)) bis(2-ethylpropane-1,3-diol) (i.e., di(trimethylolpropane)), 4.953 parts by weight of adipic acid, and 21.549 parts by weight of 2-(naphthalen-2-yloxy)acetic acid, and 5.037 parts by weight of mixed Xylenes. The contents of the reactor were mixed and heated to 230° C. By-product water was removed as it was generated, and the temperature was maintained above 200° C. for about 5 hours, then as much of the xylenes was removed as possible and the reaction product was cooled to 85° C. To the reactor was then added 16.418 parts by weight of molten hexahydrophthalic anhydride (60° C.) and 1.484 parts by weight ethyl 3-ethoxypropionate. The contents of the reactor were stirred and heated to 115° C. After the exotherm peaked (keeping the temperature below 150° C.), the contents of the reactor were heated to 145° C. The temperature was maintained at 145° C. for about 120 minutes. Maintaining agitation and keeping the temperature between 140-148° C., 15.080 parts by weight of Cardura™ E10-P (glycidyl ester of Versatic™ acid, a neo-carboxylic acid in which the carbon alpha to the carboxyl group bears a methyl group and two hydrocarbyl groups having a combined seven carbons atoms obtained from Momentive, Columbus, Ohio) was added over about 30 minutes, followed by a flush of 0.794 parts by weight ethyl 3-ethoxypropionate. The reaction mixture was held with agitation at 145° C. for about 3 hours, then cooled to about 100° C. Then the reaction mixture was further reduced with 13.167 parts by weight of Ethylene glycol monobutyl ether and cooled to about 65° C. Then 2.895 parts by weight of Dimethylethanolamine were added to the reaction mixture, followed by the addition of 1.045 parts by weight of Ethylene glycol monobutyl ether. The reaction mixture was then allowed to mix for about an hour before pouring off.

### Example 2. Synthesis of Another Water-Reducible Aryl-Modified Hyperbranched Polyol

A reactor was charged with 13.925 parts by weight of 2,2′-(oxybis(methylene)) bis(2-ethylpropane-1,3-diol) (i.e., di(trimethylolpropane)), 15.754 parts by weight of C₃₆ dimer fatty acid, (Pripol 1009™ obtained from Croda Uniqema, Inc. Chicago, Ill.), and 5.533 parts by weight of mixed Xylenes. The contents of the reactor were mixed and heated to 230° C. By-product water was removed as it was generated, and the temperature was maintained above 200° C. for about 2 hours. Afterward, the reaction mixture was cooled to below 100° C. and 16.923 parts by weight of 2-(naphthalen-2-yloxy)acetic acid and 5.070 parts by weight of mixed Xylenes were added to the reaction mixture. The contents of the reactor were again mixed and heated to 230° C. By-product water was removed as it was generated, and the temperature was maintained above 200° C. for about 6 hours. Then as much of the xylenes was removed as possible and the reaction product was cooled to about 95° C. To the reactor was then added 12.907 parts by weight of molten hexahydrophthalic anhydride (60° C.) and 1.204 parts by weight ethyl 3-ethoxypropionate. The contents of the reactor were stirred and heated to 115° C. After the exotherm peaked (keeping the temperature below 150° C.), the contents of the reactor were heated to 145° C. The temperature was maintained at 145° C. for about 180 minutes Maintaining agitation and keeping the temperature between 140-148° C., 11.865 parts by weight of Cardura™ E10-P (glycidyl ester of Versatic™ acid, a neo-carboxylic acid in which the carbon alpha to the carboxyl group bears a methyl group and two hydrocarbyl groups having a combined seven carbons atoms obtained from Momentive, Columbus, Ohio) was added over about 20 minutes, followed by a flush of 0.648 parts by weight ethyl 3-ethoxypropionate. The reaction mixture was held with agitation at 145° C. for about 3 hours, then cooled to about 75° C. To the reactor was then added 2.755 parts by weight of Dimethylethanolamine and the reaction mixture was further reduced with 13.416 parts by weight of Ethylene glycol monobutyl ether and cooled to about 65° C. The reaction mixture was then allowed to mix for about an hour before pouring off.

### Example 3. Synthesis of an Aryl-Modified Hyperbranched Polyol Having Poly-Lactone Stabilization Chains

A reactor was charged with 3.258 wt % of 2,2′-(oxybis(methylene))bis(2-(hydroxymethyl)propane-1,3-diol) (i.e., di(pentaerythritol)), 0.926 wt % of adipic acid, 10.243 wt % of 2-naphthoxyacetic acid, 0.027 wt % of DBTO, and 3.812 wt % of mixed xylenes. The contents of the reactor were mixed and heated to 230° C. By-product water was removed as it was generated, and the temperature was maintained above 200° C. for about 3 hours, removing all water. Then, the contents of the reactor were cooled to less than 140° C. Then, the reaction product was reduced and further cooled to about 100° C. with 5.856 wt % of mixed xylenes.

To the reactor, which was at 100° C., 1.874 wt % of phthalic anhydride was added followed by a flush with 0.506 wt % of mixed xylenes. The contents of the reactor were stirred and heated to 120° C. After the exotherm peaked (keeping the temperature below 140° C.), the contents of the reactor were heated to 140° C. for 120 minutes, then cooled to 100° C.

To the reactor, which was at 100° C., 3.162 wt % of 2,2′-(oxybis(methylene)) bis(2-ethylpropane-1,3-diol) (i.e., di(trimethylolpropane)) was added followed by a flush with 0.314 wt % of mixed xylenes. The contents of the reactor were mixed and heated to 230° C. By-product water was removed as it was generated, and the temperature was maintained above 200° C. for about 6 hours, removing all water and as much xylene as possible. The contents of the reactor were then cooled to about 150° C.

To the reactor, which was at 150° C., 0.016 wt % of TEGOKAT® 129, (Tin(II) 2-ethylhexanoate), was added followed by 21.690 wt % of ε-Caprolactone added over about 60 minutes followed by a flush with 1.022 wt % of n-butyl acetate. The contents of the reactor held at 150° C. for about 2 hours, (until NV indicated complete conversion). Once the conversion was complete, the reaction mixture was cooled and reduced with 47.293 wt % of n-butyl acetate.

### Example 4. Synthesis of Another Aryl-Modified Hyperbranched Polyol Having Poly-Lactone Stabilization Chains

A reactor was charged with 1.029 wt % of 2,2′-(oxybis(methylene))bis(2-(hydroxymethyl)propane-1,3-diol) (i.e., di(pentaerythritol)), 0.296 wt % of adipic acid, 3.243 wt % of 2-naphthoxyacetic acid, 0.010 wt % of DBTO, and 3.403 wt % of mixed xylenes. The contents of the reactor were mixed and heated to 230° C. By-product water was removed as it was generated, and the temperature was maintained above 200° C. for about 3 hours, removing all water. Then, the contents of the reactor were cooled to about 105° C. Then, the reaction product was reduced with 1.943 wt % of mixed xylenes.

To the reactor, which was at about 100° C., 0.592 wt % of phthalic anhydride was added followed by a flush with 0.351 wt % of mixed xylenes. The contents of the reactor were stirred and heated to 120° C. After the exotherm peaked (keeping the temperature below 140° C.), the contents of the reactor were heated to 140° C. and held there for about 120 minutes, then cooled to below 100° C.

To the reactor, which was less than 100° C., 0.999 wt % of 2,2′-(oxybis(methylene)) bis(2-ethylpropane-1,3-diol) (i.e., di(trimethylolpropane)) was added followed by a flush with 0.201 wt % of mixed xylenes. The contents of the reactor were mixed and heated to 230° C. By-product water was removed as it was generated, and the temperature was maintained above 200° C. for about 7 hours, removing all water and as much xylene as possible. The contents of the reactor were then cooled to about 120° C.

To the reactor, which was at about 120° C., 0.100 wt % of TEGOKAT® 129, (Tin(II) 2-ethylhexanoate), was added followed by a mixture of 27.402 wt % of ε-Caprolactone with 6.008 wt % of δ-Valerolactone added over about 60 minutes followed by a flush with 0.562 wt % of n-butyl acetate. The contents of the reactor held at between 110 and 120° C. during the mixed lactone monomer addition and for about 7 hours afterward.

The reaction product was then reduced by the addition of 8.353 wt % n-Butyl acetate and transferred to a larger vessel for further reduction. Once transferred, the reaction mixture was further reduced with 45.508 wt % of n-butyl acetate and cooled to about 60° C. for filtration and pour off.

### Example 5. Synthesis of Another Aryl-Modified Hyperbranched Polyol Having Poly-Lactone Stabilization Chains

A reactor was charged with 14.046 wt % of tripentaerythritol, 0.105 wt % of dibutyltin oxide, 41.929 wt % of 2-naphthoxyacetic acid, and 8.368 wt % of mixed xylenes. The contents of the reactor were mixed and heated to 230° C. By-product water was removed as it was generated, and the temperature was maintained above 200° C. for about 3 hours, removing all the water generated and as much of the xylene as practical. The contents of the reactor were cooled to below 130° C. Then, 35.535 wt % n-butyl acetate was added to the reactor to reduce and further cool the contents. The contents of the reactor were cooled and isolated as intermediate product X at 59.1% NV.

A reactor was charged with 13.466 wt % of intermediate product X (at 59.1% NV in n-Butyl acetate), 0.046 wt % of tin(II) 2-ethylhexanoate, and 5.188 wt % of ethyl 3-ethoxy propionate. The contents of the reactor were mixed and heated to 150° C. Solvent, (predominantly n-butyl acetate), was distilled away from the reactor to permit the contents to reach 150° C.

To the reactor, after the reactor contents have been stabilized at 150° C., a mixture of 5.199 wt % d-valerolactone and 23.727 wt % e-caprolactone, (20/80 relative percentages), were added over 30 to 60 minutes and flushed into the reactor by 1.153 wt % n-butyl acetate. The reactor contents were maintained at 150° C. for about 180 minutes, after which the contents were cooled to below 130° C. After the contents were cooled below 130° C., they were transferred to a larger vessel for further reduction. Once transferred, the reaction mixture was further reduced to about 40% NV with 51.220 wt % of n-butyl acetate and cooled to about 60° C. for filtration and pour off.

The structure of the aryl-modified hyperbranched polyol according to Example 5 is shown below:

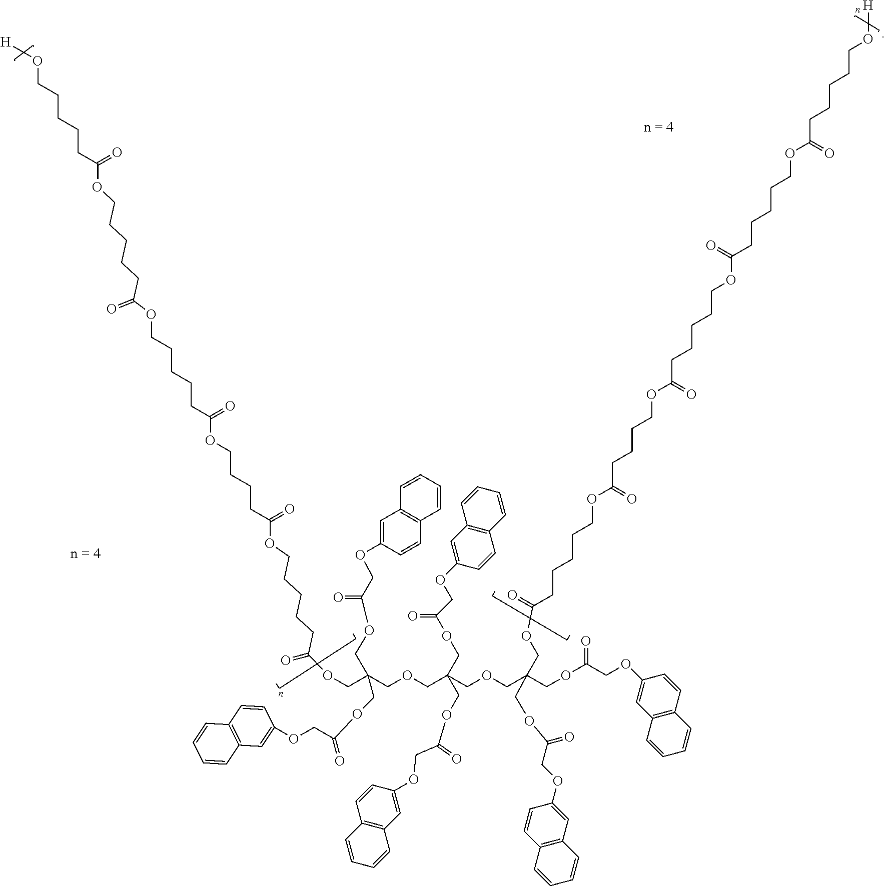

### Example 6. Synthesis of Another Aryl-Modified Hyperbranched Polyol Having Poly-Lactone Stabilization Chains

A reactor was charged with 72.133 wt % of pentaerythritol ethoxylate (3/4 EO/OH), 19.293 wt % of adipic acid, and 8.574 wt % of mixed xylenes. The contents of the reactor were mixed and heated to 230° C. By-product water was removed as it was generated, and the temperature was maintained above 200° C. for about 6 hours, removing all the water generated and as much of the xylene as practical. Then, the contents of the reactor were cooled and isolated as intermediate product A at 99.6% NV.

A reactor was charged with 25.024 wt % of intermediate product A (at 99.6% NV in Xylene), 31.338 wt % of 2-Naphthoxyacetic acid, and 7.976 wt % of mixed xylenes. The contents of the reactor were mixed and heated to 230° C. By-product water was removed as it was generated, and the temperature was maintained above 200° C. for about 3 hours, removing all the water generated and as much of the xylene as practical. The contents of the reactor were cooled to below 130° C. Then, 35.663 wt % n-butyl acetate was added to the reactor to reduce and further cool the contents. The contents of the reactor were cooled and isolated as intermediate product B at 60.3% NV.

A reactor was charged with 15.088 wt % of intermediate product B (at 60.3% NV in n-Butyl acetate), 0.029 wt % of tin(II) 2-ethylhexanoate, and 4.452 wt % of ethyl 3-ethoxy propionate. The contents of the reactor were mixed and heated to 150° C. solvent, (predominantly n-Butyl acetate), was distilled away from the reactor to permit the contents to reach 150° C.

To the reactor, after the reactor contents have been stabilized at 150° C., a mixture of 5.206 wt % d-valerolactone and 23.734 wt % e-caprolactone, (20/80 relative percents), were added over 30 to 60 minutes and flushed into the reactor by 0.754 wt % n-butyl acetate. The reactor contents were maintained at 150° C. for about 180 minutes, after which the contents were cooled to below 130° C. After the contents were cooled below 130° C., they were transferred to a larger vessel for further reduction. Once transferred, the reaction mixture was further reduced to about 40% NV with 50.737 wt % of n-butyl acetate and cooled to about 60° C. for filtration and pour off.

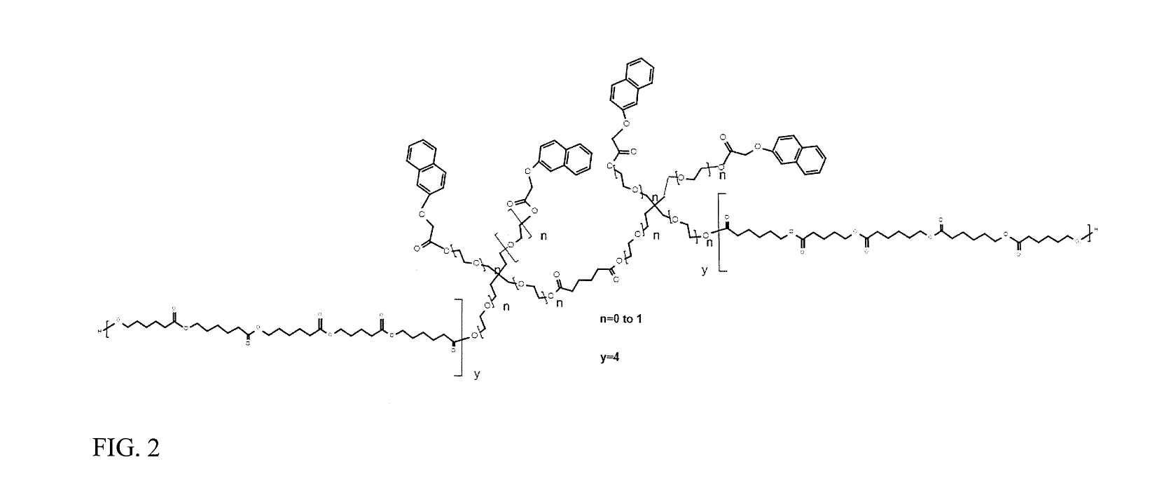

The structure of the aryl-modified hyperbranched polyol according to Example 6 is shown in FIG. 2:

### Evaluation of Aryl-Modified Hyperbranched Polyols

The embodiment as described in Example 1 was made into a low VOC, (˜246 g/L), water-reducible basecoat comprising Xirallic F60-50 pigment and black pigment and was compared to a commercial, (˜469 g/L VOC), water-reducible basecoat comprising Xirallic F60-50 pigment and black pigment sold under the name GLASURIT LINE-90. The basecoats were applied on both scuffed and unscuffed U32AD500 e-coated panels from ACT. Two coats of basecoat were applied followed by a commercial clearcoat after the basecoat had flashed matte.

For the powerwash adhesion test, the coated panels were aged for 24 hours at 140° F. and then powerwash adhesion tested before and after aging 240 hours in a humidity cabinet. The powerwash adhesion test involved making an X scribe cut on the coated panel, spraying 60° C. water at the scribe cut from 10 cm distance for 1 minute under 10-15 psi, and then measuring the amount of delamination caused. These results are shown in FIG. 3.

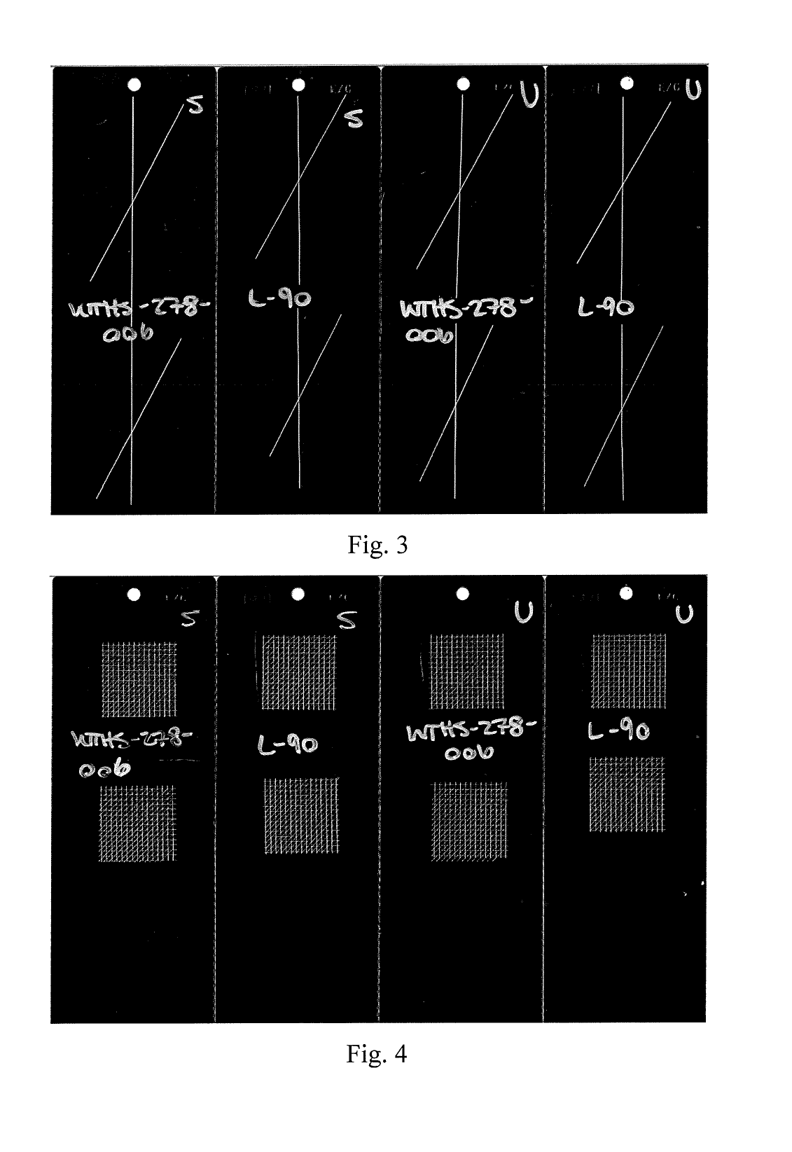

For the Ford adhesion test, the coated panels were aged for 24 hours at 140° F. and then the cross-hatched adhesion tested before and after aging 240 hours of Ford water immersion aging. The Ford immersion adhesion test involves cutting, (both before and after immersing the coated panel in 100° F. water for 240 hours), a 14×14 cross-hatch scribe with a diagonal scribe through the squares into the coated panel, then pulling adhesion tests on the scribed area and assessing any delamination experienced. Fresh scribes are made for each test. These results are shown in FIG. 4.

In each case, the recovery time before testing and after exposure was 30 minutes.

The performance of the coating made using Example 1 is improved over the performance of the commercial counterpart (GLASURIT LINE-90) as shown in FIG. 3 (powerwash adhesion test) and FIG. 4 (Ford adhesion test), and summarized in Table 1 below:

TABLE 1

| | Powerwash | | Adhesion | |
|---|---|---|---|---|
| | (mm delamination) | | (0-10 scale) | |
| | | 240-hr | | 240-hr |
| | initial | humidity | initial | immersion |
| Unscuffed E-Coat | | | | |
| commercial - 469 g/L VOC | 2.5 mm | 6.5 mm | 10 | 10 |
| with Example 1 - 246 g/L | 0 mm | 0 mm | 10 | 10 |
| VOC | | | | |
| Scuffed E-Coat | | | | |
| commercial - 469 g/L VOC | 3.5 mm | 4.5 mm | 10 | 10 |
| with Example 1 - 246 g/L | 0 mm | 0 mm | 10 | 10 |
| VOC | | | | |

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

The following claims are fully described and enabled by the above description, and are incorporated therein as a part thereof.

1. A coating composition, comprising:
an aryl-modified hyperbranched polyol obtained by a process comprising:
(a) reacting a portion of hydroxyl functional groups on a core comprising hydroxyl functional groups with an aromatic carboxylic acid of the formula (I): 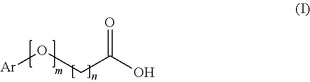
wherein: Ar is an optionally substituted aryl group; m is 0 or 1; and n is 0 or an integer from 1 to 8,
to obtain a first intermediate product still comprising at least one hydroxyl functional group; and then,
(b1) reacting the remaining hydroxyl functional groups on the first intermediate product with lactone(s), to obtain an aryl-modified hyperbranched polyol with polylactone chain extensions for solventborne coating compositions; or
(b2) reacting the remaining hydroxyl functional groups on the first intermediate product with a cyclic carboxylic acid anhydride to form a carboxylic acid-functional second intermediate product comprising carboxylic acid functional groups; and then,
(c1) reacting essentially all the carboxylic acid functional groups of the second intermediate product with an epoxide-functional compound having one epoxide group, to obtain an aryl-modified hyperbranched polyol comprising hydroxyl functional groups which is useful for solventborne coating compositions; or
(c2) reacting a portion of the carboxylic acid functional groups of the second intermediate product with an epoxide-functional compound having one epoxide group, to obtain an aryl-modified hyperbranched polyol comprising carboxylic acid functional groups and hydroxyl functional groups which is useful for waterborne coating compositions; and optionally,
(c2a) reacting hydroxyl functional groups of the aryl-modified hyperbranched polyol comprising carboxylic acid functional groups and hydroxyl functional groups with lactone(s), to obtain an aryl-modified hyperbranched polyol comprising carboxylic acid functional groups and polylactone chain extensions for waterborne coating compositions and; or
(c3) reacting essentially all the carboxylic acid functional groups of the second intermediate product with a polyol comprising at least three hydroxyl groups, to obtain a third intermediate product comprising hydroxyl functional groups; and then,
(d1) reacting the third intermediate product with a lactone(s), to obtain an aryl-modified hyperbranched polyol with polylactone chain extensions for solventborne coating compositions; or
(d2) reacting a portion of the hydroxyl functional groups on the third intermediate product with a cyclic carboxylic acid anhydride to form a fourth intermediate product comprising carboxylic acid functional groups and hydroxyl functional groups which is useful for waterborne coating compositions; and optionally,
(d2a) reacting the hydroxyl functional groups of the fourth intermediate product with lactone(s), to obtain an aryl-modified hyperbranched polyol comprising carboxylic acid functional groups and polylactone chain extensions for waterborne coating compositions and with polylactone chain extensions; or
(d3) reacting the remaining hydroxyl functional groups on the third intermediate product with a cyclic carboxylic acid anhydride to form a fourth intermediate product comprising carboxylic acid functional groups; and then,
(e1) reacting essentially all the carboxylic acid functional groups of the fourth intermediate product with an epoxide-functional compound having one epoxide group, to obtain an aryl-modified hyperbranched polyol comprising hydroxyl functional groups which is useful for solventborne coating compositions; and optionally,
(e1a) reacting hydroxyl functional groups of the aryl-modified hyperbranched polyol comprising carboxylic acid functional groups and hydroxyl functional groups with lactone(s), to obtain an aryl-modified hyperbranched polyol comprising carboxylic acid functional groups and polylactone chain extensions for waterborne coating compositions; or
(e2) reacting a portion of the carboxylic acid functional groups of the fourth intermediate product with an epoxide-functional compound having one epoxide group, to obtain an aryl-modified hyperbranched polyol comprising carboxylic acid functional groups and hydroxyl functional groups which is useful for waterborne coating compositions; and optionally,
(e2a) reacting hydroxyl functional groups of the aryl-modified hyperbranched polyol comprising carboxylic acid functional groups and hydroxyl functional groups with lactone(s), to obtain an aryl-modified hyperbranched polyol comprising carboxylic acid functional groups and polylactone chain extensions for waterborne coating compositions,
wherein the core comprising hydroxyl functional groups is a polyol comprising at least three hydroxyl groups or a reaction product obtained by reacting a polyol comprising at least three hydroxyl groups with an aliphatic dicarboxylic acid having from 6 to 36 carbon atoms, an aromatic dicarboxylic acid having from 8 to 20 carbon atoms, an esterifiable derivative of the aliphatic or aromatic dicarboxylic acid, or a polyurethane diacid according to the following formula: 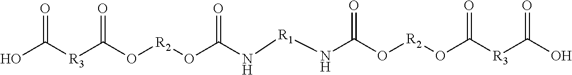
wherein:
R₁ and R₂ are each a C₂-C₁₆ aliphatic group; and
R₃ is a C₂-C₂₂ substituted or unsubstituted aliphatic, alicyclic, or aromatic group.

2. The coating composition of claim 1, wherein the aromatic carboxylic acid has formula (II) or (III): 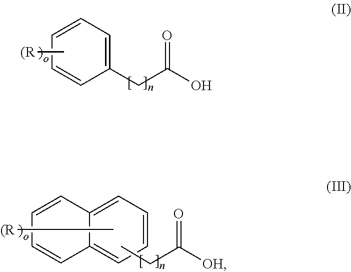
wherein:
n is an integer from 1 to 4;
o is 0 or an integer from 1 to 3; and
each R, if present, is independently selected from the group consisting of a straight or branched chain alkyl of 1 to 6 carbon atoms, an alkoxy of 1 to 6 carbon atoms, an alkoxy alkyl in which the alkyl moiety has 1 to 6 carbon atoms, halo, phenyl, and hydroxy.

3. The coating composition of claim 2, wherein the aromatic carboxylic acid is at least one selected from the group consisting of:

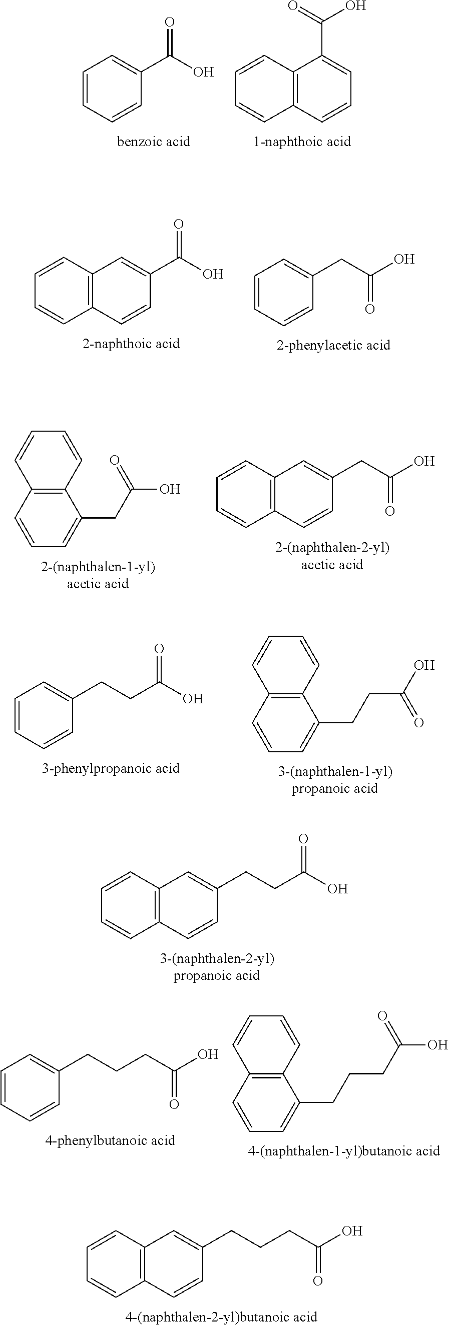

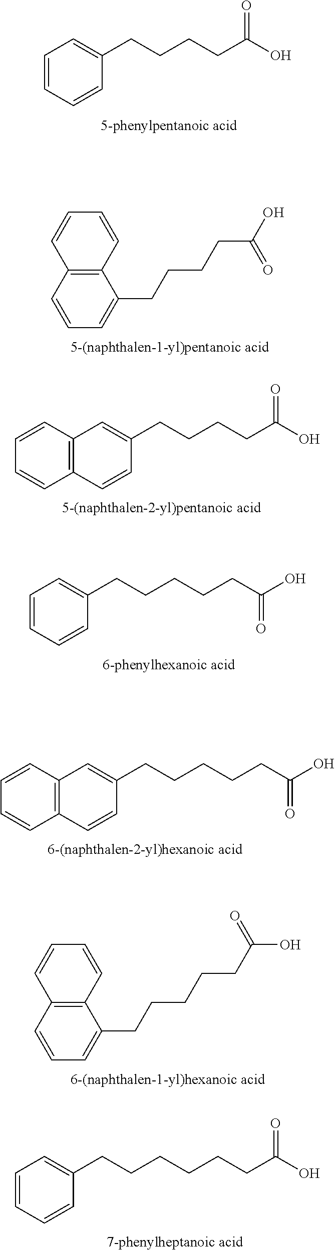

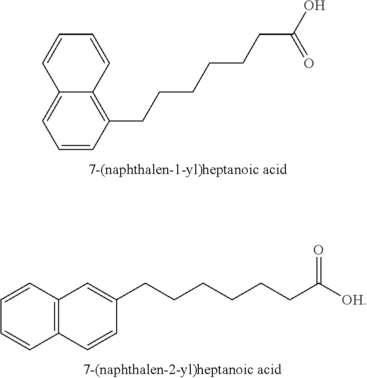 [SMILES: O=C(O)CCCCCCc1cccc2ccccc12 ; O=C(O)CCCCCCc1ccc2ccccc2c1]

4. The coating composition of claim 1, wherein the aromatic carboxylic acid has formula (IV) or (V): 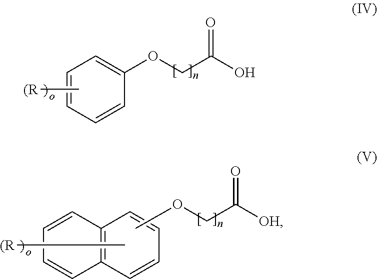
wherein:
n is 0 or an integer from 1 to 8;
o is 0 or an integer from 1 to 3; and
each R, if present, is independently selected from the group consisting of a straight or branched chain alkyl of 1 to 6 carbon atoms, an alkoxy of 1 to 6 carbon atoms, an alkoxy alkyl in which the alkyl moiety has 1 to 6 carbon atoms, halo, phenyl, and hydroxy.

5. The coating composition of claim 4, wherein the aromatic carboxylic acid is at least one selected from the group consisting of:

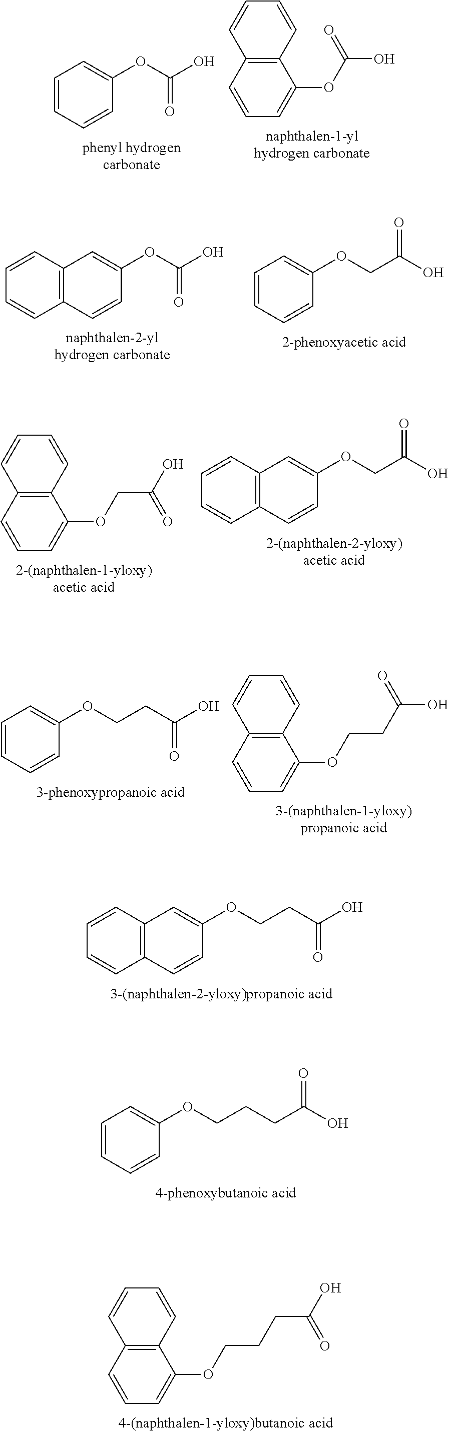

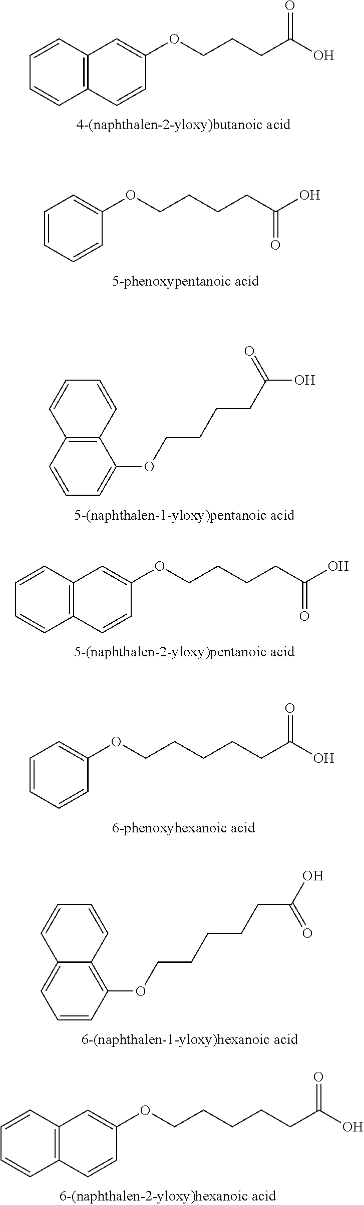

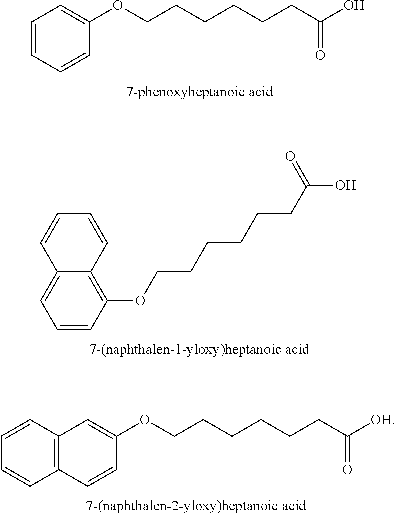 [SMILES: O=C(O)CCCCCCOc1ccccc1 ; O=C(O)CCCCCCOc1cccc2ccccc12 ; O=C(O)CCCCCCOc1ccc2ccccc2c1]

6. The coating composition of claim 1, further comprising water,
wherein the aryl-modified hyperbranched polyol is obtained by: the reacting (b1); the reacting (b2) and the reacting (e1); the reacting (b2), the reacting (c3), and the reacting (d1);
the reacting (b2), the reacting (c3), the reacting (d3), and the reacting (e1); or the reacting (b2), the reacting (c3), the reacting (d3), the reacting (e1), and the reacting (e1a).

7. The coating composition of claim 6, wherein the aryl-modified hyperbranched polyol is obtained by the reacting (b2), the reacting (c3), and the reacting (d1).

8. The coating composition of claim 6, wherein the aryl-modified hyperbranched polyol has formula (VIII-A), formula (VIII-B), or formula (X): 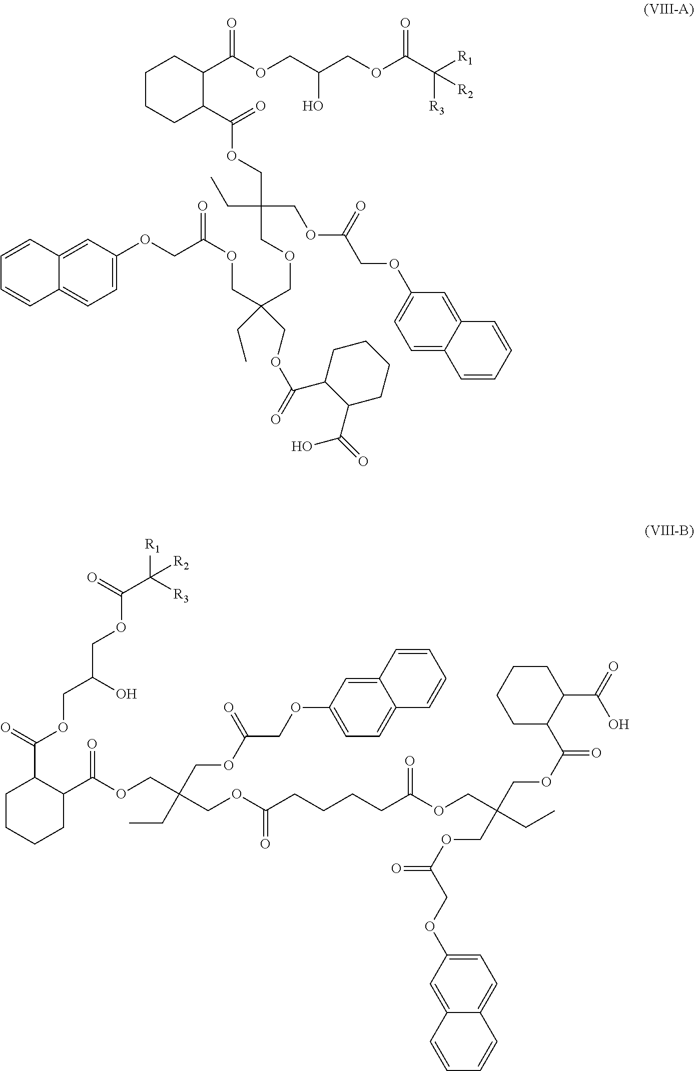 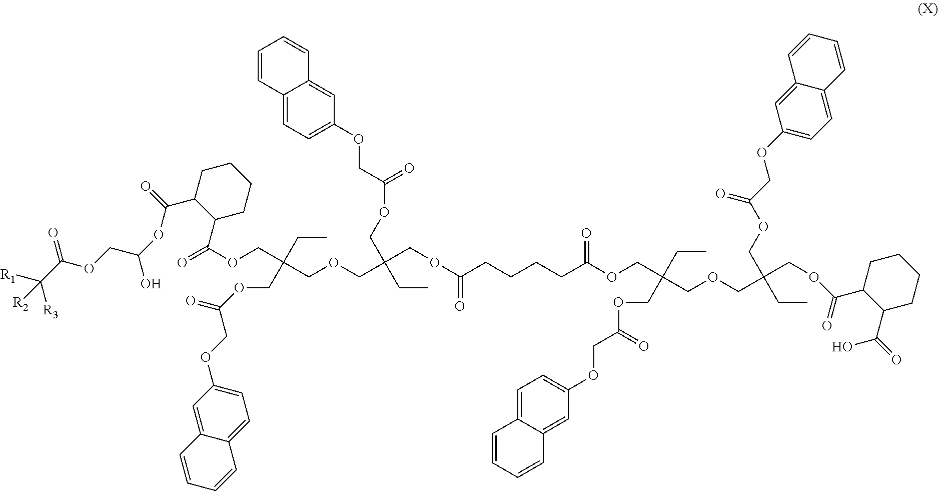
wherein R₁R₂R₃ is neodecanoate.

9. The coating composition of claim 1, further comprising an organic solvent,
wherein the aryl-modified hyperbranched polyol is obtained by: the reacting (b2) and the reacting (c2) and optionally the reacting (c2a); the reacting (b2), the reacting (c3), the reacting (d2), and optionally the reacting (d2a); or the reacting (b2), the reacting (c3), the reacting (d3), the reacting (e2), and optionally the reacting (e2a).

10. The coating composition of claim 9, wherein the aryl-modified hyperbranched polyol is obtained by the reacting (b2), the reacting (c3), the reacting (d2), and optionally the reacting (d2a).

11. The coating composition of claim 9, wherein the aryl-modified hyperbranched polyol has formula (XVI) or (XVII): 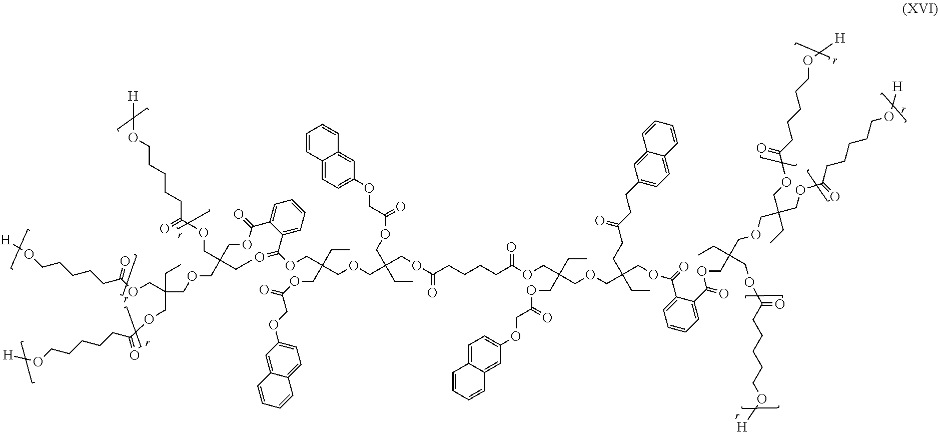 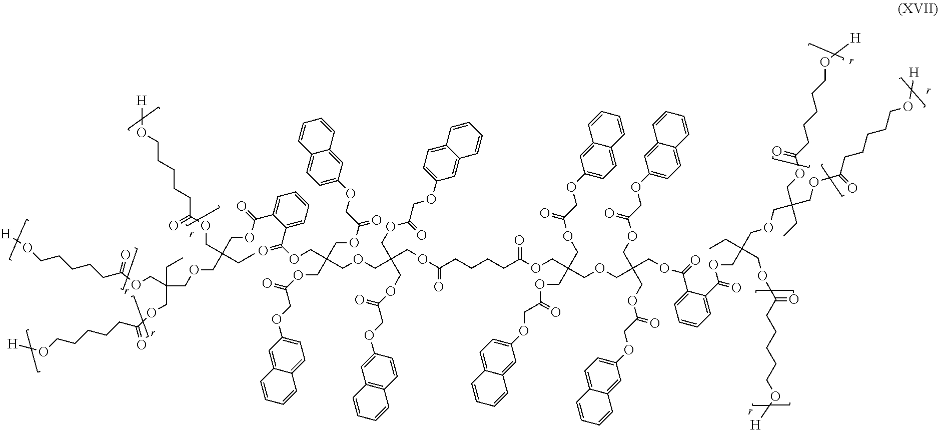
wherein r is from 1 to 30.

12. The coating composition of claim 1, wherein the core comprising hydroxyl functional groups is a polyol selected from the group consisting of trimethylolpropane, pentaerythritol, di(trimethylolpropane), tri(pentaerythritol), and di(pentaerythritol).

13. The coating composition of claim 1, wherein the core comprising hydroxyl functional groups is a polyester polyol comprising, in reacted form: a polyol selected from the group consisting of trimethylolpropane, pentaerythritol, di(trimethylolpropane), tri(pentaerythritol), and di(pentaerythritol); and an aliphatic dicarboxylic acid having from 6 to 36 carbon atoms or an aromatic dicarboxylic acid having from 8 to 20 carbon atoms.

14. The coating composition of claim 1, wherein the core comprising hydroxyl functional groups is a polyurethane polyester polyol comprising, in reacted form: a polyol selected from the group consisting of trimethylolpropane, pentaerythritol, di(trimethylolpropane), tri(pentaerythritol), and di(pentaerythritol); and a polyurethane diacid according to the following formula: 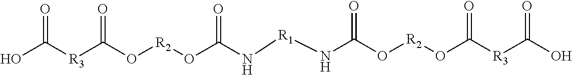
wherein:
R₁ and R₂ are each a C₂-C₁₆ aliphatic group; and
R₃ is a C₂-C₂₂ substituted or unsubstituted aliphatic, alicyclic, or aromatic group.

15. A compound of formula (VII-A) or (VII-B): 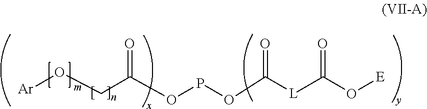
wherein:
P is a C₃-C₂₀ polyol moiety bonded to x groups of formula (I) and y groups of formula —C(O)—L—C(O)—O—E through x+y —OH functionalities;
x is an integer from 1 to 7;
y is an integer from 2 to 7;
with the proviso that the sum of x+y is from 3 to 8;
each L is independently a C₂-C₂₂ substituted or unsubstituted aliphatic, alicyclic, or aromatic moiety;
E is hydrogen or —C(OH)R₅, where R₅ is a substituted or unsubstituted C₁-C₂₂ aliphatic, alicyclic, or aromatic moiety, with the proviso that at least one E is —C(OH)R₅ and at least one E is hydrogen;
each Ar is an optionally substituted aryl group, which is a monocyclic or condensed polycyclic aromatic hydrocarbon group;
each m is 0 or 1; and
each n is 0 or an integer from 1 to 8; and 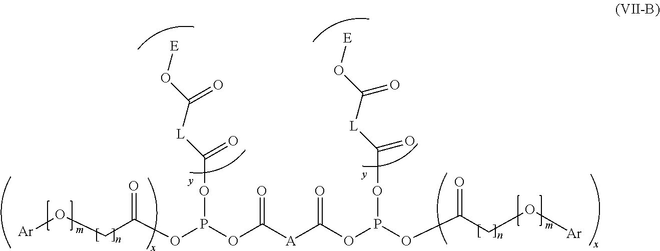
wherein:
A is C₄-C₃₄ aliphatic moiety, a C₆-C₁₈ aromatic moiety, or a moiety having the following formula: 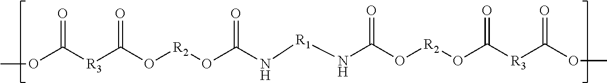
wherein:
R₁ and R₂ are each a C₃-C₈ aliphatic group; and
R₃ is a C₆-C₁₈ substituted or unsubstituted aliphatic, alicyclic, or aromatic group;
each P is independently a C₃-C₂₀ polyol moiety bonded to x groups of formula (I) and y groups of formula —C(O)—L—C(O)—O—E through x+y —OH functionalities;
each x is an integer from 1 to 6;
each y is an integer from 1 to 6;
with the proviso that the sum of x+y on each polyol P is from 2 to 7;
each L is independently a C₂-C₂₂ substituted or unsubstituted aliphatic, alicyclic, or aromatic moiety;
each E is hydrogen or —C(OH)R₅, where R₅ is a substituted or unsubstituted C₁-C₂₂ aliphatic, alicyclic, or aromatic moiety, with the proviso that at least one E is —C(OH)R₅ and at least one E is hydrogen;
each Ar is an optionally substituted aryl group, which is a monocyclic or condensed polycyclic aromatic hydrocarbon group;
each m is 0 or 1; and
each n is 0 or an integer from 1 to 8.

16. The compound of claim 15, having formula (VIII-A) or (VIII-B): 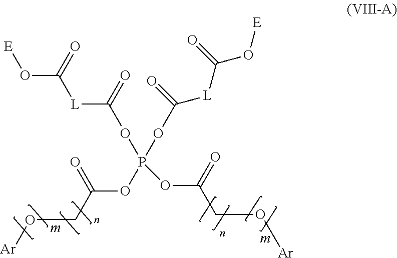
wherein:
P is a tetrol;
each Ar is an optionally substituted phenyl or naphthyl group;
each m is 0 or 1; and
each n is 0 or an integer of 1 to 3; and 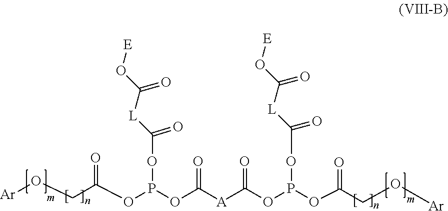
wherein:
each P is a triol;
each Ar is an optionally substituted phenyl or naphthyl group;
each m is 0 or 1; and
each n is 0 or an integer of 1 to 3.

17. A compound having formula (XI-A) or (XI-B): 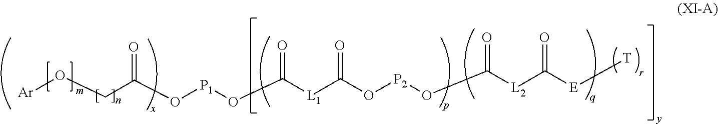
wherein:
P₁ is a C₃-C₂₀ polyol moiety bonded to x groups of formula (I) and y groups of formula —{C(O)—L₁—C(O)—O—P₂—O}ₚ—{C(O)—L₂—C(O)—E}_{q}—(T)ᵣ through x+y —OH functionalities;
x is an integer from 1 to 8;
y is an integer from 1 to 8;
with the proviso that the sum of x +y is from 3 to 10;
each L₁ and each L₂ is independently a C₂-C₂₂ substituted or unsubstituted aliphatic, alicyclic, or aromatic moiety;
each P₂ is independently a C₃-C₂₀ polyol moiety bonded to q groups of —{C(O)—L₂—C(O)—E} through q —OH functionalities or to r groups of T through r —OH functionalities;
each E is —C(R₅)—O— where R₅ is a substituted or unsubstituted C₁-C₂₂ aliphatic, alicyclic, or aromatic moiety;
p is 0 or 1;
when p is 0, q is 0 or 1 and r is 1;
when p is 1, q is 0 and r is an integer from 2 to 7 or q is an integer from 2 to 7 and r is 1;
each T is (poly)lactone chain moiety having from 1 to 30 lactone units;
each Ar is an optionally substituted aryl group, which is a monocyclic or condensed polycyclic aromatic hydrocarbon group;
each m is 0 or 1; and
each n is 0 or an integer from 1 to 8; and 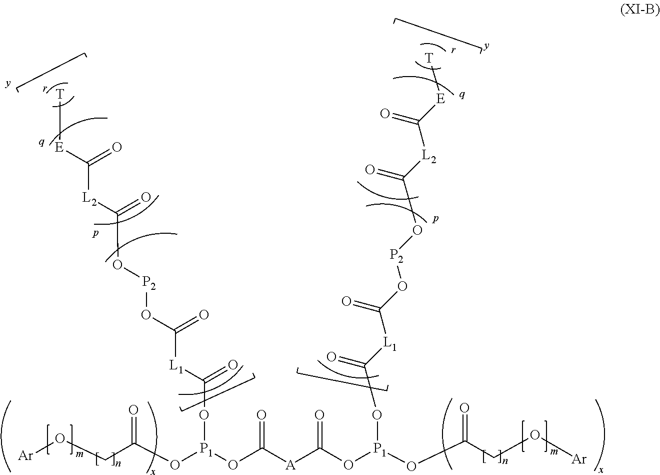
wherein:
A is C₄-C₃₄ aliphatic moiety, a C₆-C₁₈ aromatic moiety, or a moiety having the following formula: 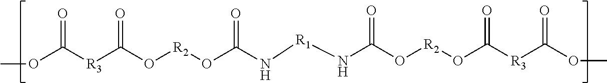
wherein:
R₁ and R₂ are each a C₃-C₈ aliphatic group; and
R₃ is a C₆-C₁₈ substituted or unsubstituted aliphatic, alicyclic, or aromatic group;
P₁ is a C₃-C₂₀ polyol moiety bonded to x groups of formula (I) and y groups of formula —{C(O)—L₁—C(O)—O—P₂—O}ₚ—{C(O)—L₂—C(O)—E}_{q}—(T)ᵣ through x+y —OH functionalities;
each x is an integer from 1 to 6;
each y is an integer from 1 to 6;
with the proviso that the sum of x+y on each polyol P₁ is from 2 to 7;
each L₁ and each L₂ is independently a C₂-C₂₂ substituted or unsubstituted aliphatic, alicyclic, or aromatic moiety;
each P₂ is independently a C₃-C₂₀ polyol moiety bonded to q groups of —{C(O)—L₂—C(O)—E} through q —OH functionalities or to r groups of T through r —OH functionalities;
each E is —C(R₅)—O— where R₅ is a substituted or unsubstituted C₁-C₂₂ aliphatic, alicyclic, or aromatic moiety;
each p is 0 or 1;
when p is 0, q is 0 or 1 and r is 1;
when p is 1, q is 0 and r is an integer from 2 to 7 or q is an integer from 2 to 7 and r is 1;
each T is (poly)lactone chain moiety having from 1 to 30 lactone units;
each Ar is an optionally substituted aryl group, which is a monocyclic or condensed polycyclic aromatic hydrocarbon group;
each m is 0 or 1; and
each n is 0 or an integer from 1 to 8.

18. The compound of claim 17, having formula (XII-A), (XIII-A), (XIV-A), or (XV-A): 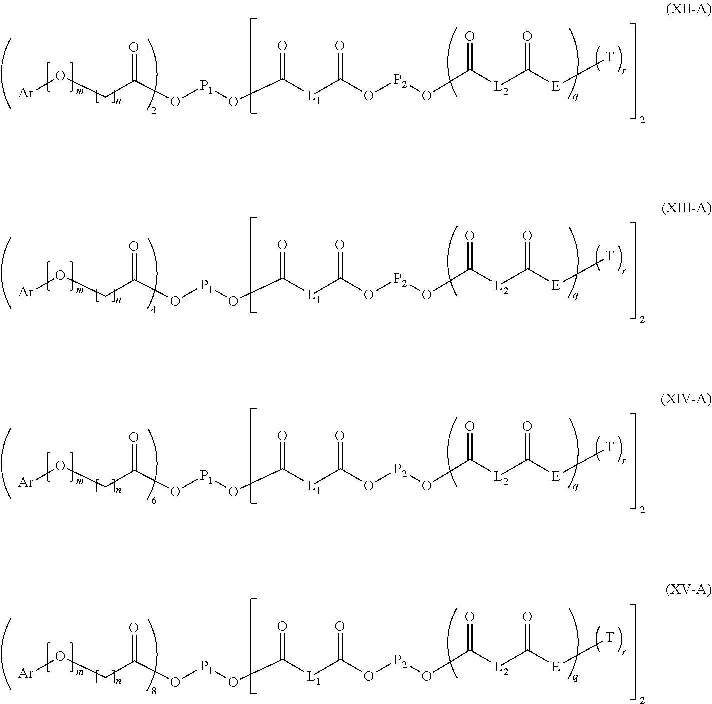
wherein:
each P₁ is a polyol having 4 —OH functionalities in formula (XII-A), 6 —OH functionalities in formulae (XIII-A), 8 —OH functionalities in formula (XIV-A), and 10 —OH functionalities in formula (XV-A);
each L₁ and each L₂ is independently a C₂-C₂₂ substituted or unsubstituted aliphatic, alicyclic, or aromatic moiety;
each P₂ is independently selected from the group of glycerol (q=2 and r=1, or q=0 and r=2), trimethylolpropane (q=2 and r=1, or q=0 and r=2), di(trimethylolpropane) (q=3 and r=1, or q=0 and r=3), pentaerythritol (q=3 and r=1, or q=0 r=3), di(pentaerythritol) (q=5 and r=1, or q=0 r=5), and tri(pentaerythritol) (q=7 and r=1, or q=0 r=7);
each E is —C(R₅)—O— where R₅ is a substituted or unsubstituted C₁-C₂₂ aliphatic, alicyclic, or aromatic moiety;
each T is (poly)lactone chain moiety having from 1 to 30 lactone units;
each Ar is an optionally substituted phenyl or naphthyl group;
each m is 0 or 1; and
each n is 0 or an integer of 1 to 3.

19. The compound of claim 18, having formula (XII-B), (XIII-B), (XIV-B), or (XV-B): 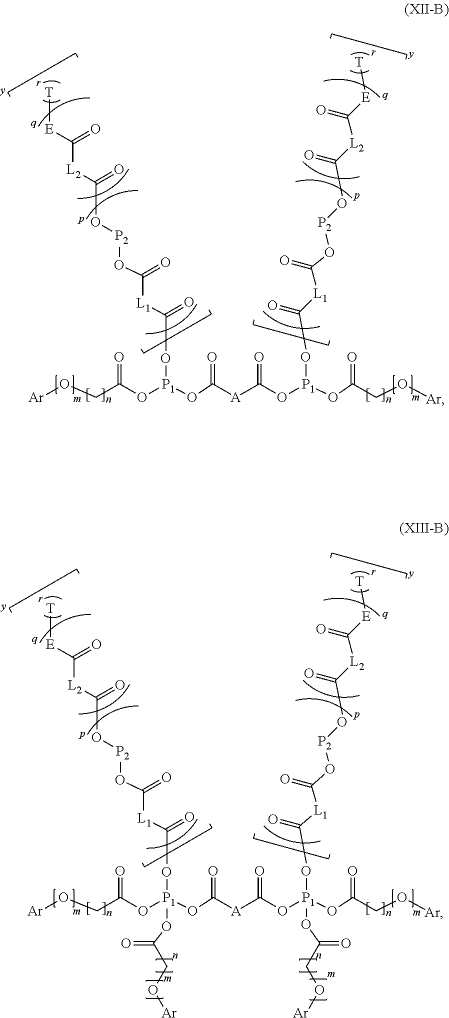 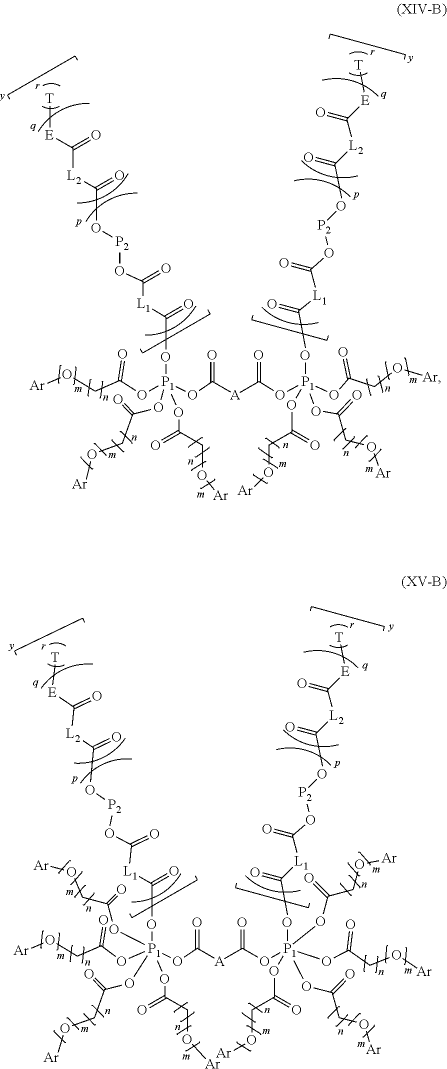
wherein:
A is C₄-C₁₀ aliphatic moiety, a C₆-C₁₂ aromatic moiety, or a moiety having the following formula: 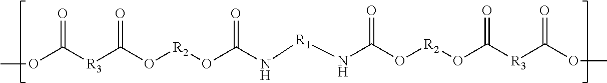
wherein:
R₁ and R₂ are each a C₃-C₈ aliphatic group; and
R₃ is a C₆-C₁₈ substituted or unsubstituted aliphatic, alicyclic, or aromatic group;
each P₁ is a polyol having 3 —OH functionalities in formula (XII-B); 4 —OH functionalities in formula (XIII-B), 5 —OH functionalities in formula (XIV-B), and 6 —OH functionalities in formula (XV-B);
each L₁ and each L₂ is independently a C₂-C₂₂ substituted or unsubstituted aliphatic, alicyclic, or aromatic moiety;
when p=1, each P₂ is independently selected from the group of glycerol (q=2 and r=1, or q=0 and r=2), trimethylolpropane (q=2 and r=1, or q=0 and r=2), di(trimethylolpropane) (q=3 and r=1, or q=0 and r=3), pentaerythritol (q=3 and r=1, or q=0 and r=3), di(pentaerythritol) (q=5 and r=1, or q=0 r=5), and tri(pentaerythritol) (q=7 and r=1, or q=0 r=7);
when p=0, q is 0 or 1 and r is 1;
each E is —C(R₅)—O— where R₅ is a substituted or unsubstituted C₁-C₂₂ aliphatic, alicyclic, or aromatic moiety;
each T is (poly)lactone chain moiety having from 1 to 30, lactone units;
each Ar is an optionally substituted phenyl or naphthyl group;
each m is 0 or 1; and
each n is 0 or an integer of 1 to 3.

20. A method, comprising:
applying the coating composition of claim 1 to a substrate, to form a coating layer on the substrate; and
curing the coating layer.
